(19) United States
(12) Patent Application Publication
(10) Pub. No.: US 2024/0409912 A1
(43) Pub. Date: Dec. 12, 2024

(54) ENGINEERED PROTEASE POLYPEPTIDES

(71) Applicants: Codexis, Inc., Redwood City, CA (US); Société des Produits Nestlé S.A., Vevey (CH)

(72) Inventors: Chinping Chng, Menlo Park, CA (US); Ruth L. Cong, Palo Alto, CA (US); Da Duan, Foster City, CA (US); Brian Ferrer, San Mateo, CA (US); Ravi David Garcia, Los Gatos, CA (US); Nikki D. Kruse, San Carlos, CA (US); Hirdesh Kumar, Redwood City, CA (US); Stephen Joshua Macaso Millet, Tracy, CA (US); Trica Windgassen, Newbury Park, CA (US); Liang Zhu, San Mateo, CA (US)

(21) Appl. No.: 18/679,281

(22) Filed: May 30, 2024

Related U.S. Application Data
(60) Provisional application No. 63/505,055, filed on May 30, 2023.

(51) Int. Cl. C12N 9/54; C12N 9/96

(52) U.S. Cl. CPC C12N 9/54; C12N 9/96; C12Y 304/21

(57) ABSTRACT

The present disclosure provides engineered protease polypeptides, recombinant polynucleotides encoding the engineered protease polypeptides, and uses of the engineered protease polypeptides in therapeutic applications.

### CROSS-REFERENCE TO RELATED APPLICATIONS

This application claims the benefit of U.S. Provisional Application 63/505,055, filed May 30, 2023, which is incorporated by reference herein.

### REFERENCE TO SEQUENCE LISTING, TABLE OR COMPUTER PROGRAM

The Sequence Listing concurrently submitted herewith as file name CX7-253US2_ST26.xml, created on May 30, 2024, with a file size of 4,736,576 bytes, is hereby incorporated by reference herein in its entirety.

### TECHNICAL FIELD

The present disclosure relates to engineered protease polypeptides, compositions thereof, polynucleotides encoding the engineered protease polypeptides, and uses of the engineered polypeptides and recombinant polynucleotides in therapeutic and other applications.

### BACKGROUND

Pancreatic exocrine insufficiency (PEI) or exocrine pancreatic insufficiency (EPI) is a condition in which the pancreas does not supply a sufficient amount of digestive enzymes needed to digest food efficiently. Following passage of food through the stomach, ingested food is converted into acidic chyme that flows in the duodenum of the small intestine, which receives, among others, pancreatic enzymes (e.g., lipase, amylase, and protease) that break down the food for absorption in the small intestine. Poor digestion of food in PEI/EPI can lead to malabsorption of fats, proteins, carbohydrates, and vitamins by the intestines, which can lead to malnutrition, changes in bone density, and increased risk of mortality. The reduction in pancreatic enzymes may arise from inadequate pancreatic stimulation of pancreatic secretion, insufficient secretion of pancreatic digestive enzymes by the pancreatic acinar cells, or outflow obstruction of the pancreatic duct, and inadequate mixing of the pancreatic enzymes with food.

PEI/EPI is often associated with pancreatitis, cystic fibrosis, celiac disease, inflammatory bowel disease (IBD), Crohn's disease, ulcerative colitis, and pancreatic cancer, all of which can lead to decreased secretion of pancreatic enzymes into the duodenum. An approved treatment for PEI/EPI is pancreatic replacement therapy (PERT), which is an orally administered cocktail of digestive enzymes amylase, lipase, and protease, mostly derived from porcine origin (e.g., Creon™, Zenpep™, Pertzye™, and Pancreaz™). However, PERT treatment may not alleviate the condition in some people due to insufficient activity of the PERT enzymes in the gastrointestinal tract and/or insufficient patient compliance with the therapy due to the significant pill burden associated with current treatment protocols. In some cases, the coefficient of fat absorption (CFA) and/or coefficient of nitrogen absorption (CNA) is inferior to that of healthy patients, resulting in weight loss and other health concerns. Thus, a need remains in the art for improved PERT treatments.

### SUMMARY

The present disclosure provides engineered protease polypeptides, recombinant polynucleotides encoding the engineered protease polypeptides, and uses of the engineered protease polypeptides for degrading target proteins and polypeptides. As provided in detail herein, in some embodiments, the protease polypeptides have been engineered to exhibit an improved property compared to the naturally occurring protease, including among others, enhanced expression, increased proteolytic activity of the active protease, increased thermostability, increased resistance against gastric proteases, increased activity at acidic pH, and increased stability at acidic pH.

In some embodiments, the present disclosure provides an engineered protease polypeptide, or a biologically active fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of SEQ ID NO: 4, 628, 948, 1126, 1368, or 1548, or to a reference sequence corresponding to SEQ ID NO: 4, 628, 948, 1126, 1368, or 1548, wherein the amino acid sequence comprises one or more substitutions relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 4 or 628, or to the reference sequence corresponding to SEQ ID NO: 4 or 628, wherein the amino acid sequence comprises one or more substitutions relative to the reference sequence corresponding to residues 135-413 of SEQ ID NO: 4, or 628, or relative to the reference sequence corresponding to SEQ ID NO: 4 or 628.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 948, 1126, 1368, or 1548, or to the reference sequence corresponding to SEQ ID NO: 948, 1126, 1368, or 1548, wherein the amino acid sequence comprises one or more substitutions relative to the reference sequence corresponding to residues 135-413 of SEQ ID NO: 4 or 628, or relative to the reference sequence corresponding to SEQ ID NO: 4 or 628.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 6-2242, or to a reference sequence corresponding to an even-numbered SEQ ID NO. of SEQ ID NOs: 6-2242, wherein the amino acid sequence comprises one or more substitutions relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution at amino acid position 11, 31, 42, 45, 50, 53, 84, 99, 100, 126, 128, 129, 130, 131, 132, 134, 135, 136, 137, 138, 139, 140, 141, 143, 145, 151, 154, 156, 157, 159, 160, 161, 162, 163, 169, 172, 173, 174, 179, 180, 184, 185, 186, 187, 188, 190, 191, 192, 193, 194, 198, 199, 212, 214, 220, 221, 222, 223, 225, 231, 232, 233, 235, 237, 238, 239, 240, 242, 243, 245, 246, 249, 250, 251, 252, 253, 254, 256, 258, 262, 263, 264, 265, 266, 267, 268, 269, 271, 273, 274, 275, 277, 278, 279, 280, 281, 283, 285, 290, 292, 293, 294, 296, 297, 300, 302, 303, 311, 312, 313, 314, 315, 316, 318, 324, 328, 336, 339, 341, 342, 343, 345, 346, 355, 358, 360, 364, 367, 368, 369, 370, 371, 372, 373, 374, 375, 377, 381, 382, 384, 386, 389, 391, 392, 401, 402, 405, 406, 409, 410, 411, 412, or 413, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or amino acid residue 11K, 31G, 42W, 45Y, 50R, 53A, 84M, 99V, 100V, 126T, 128G/I/K/L/P/R/S/T/V, 129E/F/H/I/K/L/R/S/T/V, 130A/F/G/N/V, 131E/P/R/T/V/Y, 132A/C/D/E/G/P/R/V/Y, 134A/C/D/E/G/I/L/M/N/P/S/T/V/W/Y, 135C/E/G/H/I/K/L/M/N/P/R/S/T/V/W/Y, 136C/G/I/M, 137A/D/N/S, 138Q, 139C/D/E/F/H/I/K/L/M/R/S, 140L, 141A/C/D/E/F/G/H/I/L/M/Q/R/S/T/V/W/Y, 143A/C/D/N/Q/S/T, 145A/C/D/E/F/G/H/I/K/L/P/Q/R/S/T/V/W, 151D/Q, 154C/D/L/R, 156C/V, 157A/C/D/E/F/G/H/I/K/L/M/N/P/Q/R/T/V/W, 159G, 160A/C/D/E/F/K/L/M/N/P/R/Q/T/V/W/Y, 161D/E/G/L/R, 162I, 163H/L, 169S, 172Q, 173F/S, 174L, 179K/S, 180H/L/M, 184A/D/G/L/M/Q/R, 185A/D/E/F/G/L/M/P/Q/R/S/T/V, 186A/R/S/T/Y, 187A, 188A/C/D/F/G/L/M/S/T/W, 190S, 191R, 192C/D/M/N, 193T, 194A/D/L/T, 198G, 199C/K/L, 212S, 214A/C/D/E/F/G/H/I/K/L/M/N/P/Q/R/T/V/W/Y, 220K/L/R, 221A/C/D/E/F/G/H/I/K/L/M/P/Q/R/T/V/W/Y, 222G, 223S, 225V, 231H/V, 232S, 233G/I/L, 235Q/R/V, 237A/G, 238Q, 239L/M, 240A/L, 242E/S, 243E/L/M/R/S/T, 245L/V, 246I/V, 249G/M/S, 250A/C/F/L/N/T, 251D/S, 252P, 253C/I/V, 254C/E, 256L/M, 258W, 262A/S, 263E/H/P/Q/R/S, 264A/C/F/I/L/N/P/R/T/V, 265C/G/R, 266H/T/Y, 267A/G/H/I/L/M/R/S/T/V/W, 268A/F/G/H/I/N/P/Q/T/V/Y, 269Q/T, 271A, 273A/C/F/L/M/S/T, 274A/G/K/L/T/V/W, 275A/V, 277D/G, 278L/N/S/V/Y, 279A/E/F/G/H/K/L/M/R/S/T/Y/V/W, 280D/K/S/T, 281C/V, 283M, 285S, 290E/G/S, 292V, 293A, 294V/W, 296M/R, 297F, 300R/V, 302G/P, 303A/V, 311A/E/D/G/K/M/Q/S, 312A/C/D/E/G/H/I/K/L/M/N/P/Q/R/T/V/W/Y, 313A/Q/S/T, 314G, 315A/C/D/E/F/G/H/I/K/L/M/Q/R/S/V/W/Y, 316K, 318N/P/R, 324A/D/E/I/R/V/W/Y, 328L/M, 336F, 339S/W, 341G, 342A/C/D/E/F/G/I/K/M/N/P/R/Q/T/V/W/Y, 343S, 345A/C/D/E/F/G/I/K/L/M/Q/R/S/V/W/Y, 346A/C/D/F/G/K/L/M/N/P/Q/R/T/V/W/Y, 355A, 358S, 360S, 364A/V, 367V, 368G/T, 369I/V/W, 370C/E/F/G/I/K/L/P/Q/R/S/V, 371L, 372A/C/F/L/R/V/Y, 373A/C/E/F/M/S/Y, 374E/G/L/R/S/W/Y, 375A/E/I/L/M/S/T/V, 377H, 381N, 382G/R/S/T, 384C, 386P/W, 389C/P, 391L/S, 392Y, 401L, 402G/*, 405L/Q, 406C/M/R/W, 409E/R/*, 410C/I/W/*, 411L/R/T/V, 412P/T/*, or 413A/C/D/S/*, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution at amino acid position 135, 137, 139, 141, 143, 157, 160, 214, 268, 273, 279, 311, 312, 315, 328, 342, 345, 346, or 372, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or amino acid residue 135C/E/G/H/I/K/L/M/N/P/R/S/T/V/W/Y, 137A/D/N/S, 139C/D/E/F/H/I/K/L/M/R/S, 141A/C/D/E/F/G/H/I/L/M/Q/R/S/T/V/W/Y, 143A/C/D/N/Q/S/T, 157A/C/D/E/F/G/H/I/K/L/M/N/P/Q/R/T/V/W, 160A/C/D/E/F/K/L/M/N/P/R/Q/T/V/W/Y, 214A/C/D/E/F/G/H/I/K/L/M/N/P/Q/R/T/V/W/Y, 268A/F/G/H/I/N/P/Q/T/V/Y, 273A/C/F/L/M/S/T, 279A/E/F/G/H/K/L/M/R/S/T/Y/V/W, 311A/E/D/G/K/M/Q/S, 312A/C/D/E/G/H/I/K/L/M/N/P/Q/R/T/V/W/Y, 315A/C/D/E/F/G/H/I/K/L/M/Q/R/S/V/W/Y, 328L/M, 342A/C/D/E/F/G/I/K/M/N/P/R/Q/T/V/W/Y, 345A/C/D/E/F/G/I/K/L/M/Q/R/S/V/W/Y, 346A/C/D/F/G/K/L/M/N/P/Q/R/T/V/W/Y, or 372A/C/F/L/R/V/Y, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution set at amino acid positions 135/141/160/311/315/372, 143/328/342/345, 139/157/268/273/312/346, or 137/139/214/279, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or substitution set at amino acid position(s) 185, 134, 129, 135, 184, 132, 186, 193, 263, 370, 45/134, 199, 368, 161, 141, 267, 179, 264, 160, 138, 131, 372, 151, 274, 128, 339, 313, 374, 314, 191, 324, 315, 375, 136, 220, 194, 231, 277, 369, 251, 180, 163, 343, 264/279, 279, 232, 141/300, 367, 266, 188, 130, 318, 265, 341, 190, 145, 126/192, 11/220, 192, 370/392, 99/278, 265/311, 84/159/265/279/311/370, 311/316, 342/370, 265/311/370, 192/311/316, 141/154/192, 265/311/316/342, 279/311/316, 141/265/279/311/342, 141/192/311/316/370, 141/265/311, 198/279, 392, 342/370/392, 141/198/265, 265/392, 184/267, 342, 312, 100/251, 141/220, 311/316/370, 99, 278, 405, 311/342/370, 141/198, 311/342, 141/311, 279/311/377/392, 186/198/311/342/370/392, 141/392, 311/370/392, 141/311/392, 311/370, 311/316/392, 265/311/392, 141/192, 311, 141/265/311/392, 192/311/370/392, 198/265/311/316/370, 141/186/265/311, or 141/198/265/311/370, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution at amino acid position 135, 137, 139, 141, 143, 145, 145, 157, 160, 214, 221, 268, 273, 279, 311, 312, 315, 315, 342, 345, 346, 402, 409, 410, 412, or 413, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or substitution set at amino acid position(s) 242, 157, 250, 373, 243, 336, 187, 240, 280, 271, 237, 386, 382, 328, 42, 391, 381, 275, 249, 239, 384, 139, 364, 346, 389, 254, 246, 345, 360, 303, 300, 269, 135/141/372, 311/315/372, 136/141/311, 141/188, 135/136, 135/141/315, 372, 135/141/160/267/372, 135/136/141/160/185/188/267/311/315, 160/185, 135/141/188/279/311, 135/136/141, 135/136/141/372, 135/141/160/185/267/279, 135/141/160/267, 141/188/311/372, 160/185/188/279/311, 136/141/279, 135/136/141/160/185/188, 141/372, 135/136/141/311, 185/311/315/372, 135/141/188, 136/185, 135/141, 135/136/141/279/315/372, 135/311/315, 141, 311/372, 188/311, 135/141/188/372, 141/160/279, 313/392, 342/392, 279/392, 128, 198/342, 313, 128/312, 50, 145/263, 313/342, 279/312, 312/392, 279/342, 128/342, 342, 263, 143, 262, 156, 169, 143/237, 136/160/185/267/311/372, 135/160/311/372, 135/141/311/315, 141/311/315, 136/141/160/185/188/311/315/372, 135/141/311/315/372, 135/141/160/185/311/315, 135/141/267/311/315/372, 135/136/141/160/311/315, 135/136/141/279, 135/141/267/279/311/315, 135/141/160, 135/141/160/311/315/372, 135/141/160/311/315, 135/136/141/188/311, 141/160/311, 135/141/160/279/311/315/372, 141/160/185/279/311/372, 135/136/141/160/315/372, 135/136/160/279/311/372, 128/279/312/342, 128/198/312/342, 263/342, 145/263/279/312/342/392, or 128/145/198/312/313/392, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution as set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or substitution set of an engineered set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to an amino acid sequence comprising at least a substitution or substitution set of an engineered protease polypeptide variant set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 948, 1126, 1368, or 1548, or to the reference sequence corresponding to SEQ ID NO: 948, 1126, 1368, or 1548.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 6-2242, or to a reference sequence corresponding to an even-numbered SEQ ID NO. of SEQ ID NOs: 6-2242.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 6-2242, or to a reference sequence corresponding to an even-numbered SEQ ID NO. of SEQ ID NOs: 6-2242, wherein the amino acid sequence comprises one or more substitutions relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 948, 1126, 1368, or 1548, or to the reference sequence corresponding to SEQ ID NO: 948, 1126, 1368, or 1548, wherein the amino acid sequence comprises one or more substitutions relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the amino acid sequence of the engineered protease polypeptide comprises at least a substitution at amino acid position 11, 31, 42, 45, 50, 53, 84, 99, 100, 126, 128, 129, 130, 131, 132, 134, 135, 136, 137, 138, 139, 140, 141, 143, 145, 151, 154, 156, 157, 159, 160, 161, 162, 163, 169, 172, 173, 174, 179, 180, 184, 185, 186, 187, 188, 190, 191, 192, 193, 194, 198, 199, 212, 214, 220, 221, 222, 223, 225, 231, 232, 233, 235, 237, 238, 239, 240, 242, 243, 245, 246, 249, 250, 251, 252, 253, 254, 256, 258, 262, 263, 264, 265, 266, 267, 268, 269, 271, 273, 274, 275, 277, 278, 279, 280, 281, 283, 285, 290, 292, 293, 294, 296, 297, 300, 302, 303, 311, 312, 313, 314, 315, 316, 318, 324, 328, 336, 339, 341, 342, 343, 345, 346, 355, 358, 360, 364, 367, 368, 369, 370, 371, 372, 373, 374, 375, 377, 381, 382, 384, 386, 389, 391, 392, 401, 402, 405, 406, 409, 410, 411, 412, or 413, or combinations thereof, wherein the amino acid positions are relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the amino acid sequence of the engineered protease polypeptide comprises at least a substitution or amino acid residue 11K, 31G, 42W, 45Y, 50R, 53A, 84M, 99V, 100V, 126T, 128G/I/K/L/P/R/S/T/V, 129E/F/H/I/K/L/R/S/T/V, 130A/F/G/N/V, 131E/P/R/T/V/Y, 132A/C/D/E/G/P/R/V/Y, 134A/C/D/E/G/I/L/M/N/P/S/T/V/W/Y, 135A/C/E/G/H/I/K/L/M/N/P/R/S/T/V/W/Y, 136C/G/I/M, 137A/D/N/S, 138Q, 139C/D/E/F/H/I/K/L/M/N/R/S, 140L, 141A/C/D/E/F/G/H/I/L/M/N/Q/R/S/T/V/W/Y, 143A/C/D/H/N/Q/S/T, 145A/C/D/E/F/G/H/I/K/L/P/Q/R/S/T/V/W, 151D/Q, 154C/D/L/R, 156C/V, 157A/C/D/E/F/G/H/I/K/L/M/N/P/Q/R/S/T/V/W, 159G, 160A/C/D/E/F/K/L/M/N/P/R/Q/S/T/V/W/Y, 161D/E/G/L/R, 162I, 163H/L, 169S, 172Q, 173F/S, 174L, 179K/S, 180H/L/M, 184A/D/G/L/M/Q/R, 185A/D/E/F/G/L/M/P/Q/R/S/T/V, 186A/R/S/T/Y, 187A, 188A/C/D/F/G/L/M/S/T/W, 190S, 191R, 192C/D/M/N, 193T, 194A/D/L/T, 198G, 199C/K/L, 212S, 214A/C/D/E/F/G/H/I/K/L/M/N/P/Q/R/T/V/W/Y, 220K/L/R, 221A/C/D/E/F/G/H/I/K/L/M/P/Q/R/T/V/W/Y, 222G, 223S, 225V, 231H/V, 232S, 233G/I/L, 235Q/R/V, 237A/G, 238Q, 239L/M, 240A/L, 242E/S, 243E/L/M/R/S/T, 245L/V, 246I/V, 249G/M/S, 250A/C/F/L/N/T, 251D/S/T, 252P, 253C/I/V, 254C/E, 256L/M, 258W, 262A/S, 263E/H/P/Q/R/S, 264A/C/F/I/L/N/P/R/T/V, 265C/G/R, 266H/T/Y, 267A/G/H/I/L/M/R/S/T/V/W, 268A/F/G/H/I/N/P/Q/S/T/V/Y, 269Q/T, 271A, 273A/C/F/L/M/S/T/V, 274A/G/K/L/T/V/W, 275A/V, 277D/G, 278L/N/S/V/Y, 279A/E/F/G/H/K/L/M/R/S/T/Y/V/W, 280D/K/S/T, 281C/V, 283M, 285S, 290E/G/S, 292V, 293A, 294V/W, 296M/R, 297F, 300R/V, 302G/P, 303A/V, 311A/E/D/G/K/M/Q/S/T, 312A/C/D/E/G/H/I/K/L/M/N/P/Q/R/S/T/V/W/Y, 313A/Q/S/T, 314G, 315A/C/D/E/F/G/H/I/K/L/M/Q/R/S/T/V/W/Y, 316K, 318N/P/R, 324A/D/E/I/R/V/W/Y, 328L/M/V, 336F, 339S/W, 341G, 342A/C/D/E/F/G/I/K/M/N/P/R/Q/S/T/V/W/Y, 343S, 345A/C/D/E/F/G/I/K/L/M/Q/R/S/V/W/Y, 346A/C/D/F/G/K/L/M/N/P/Q/R/S/T/V/W/Y, 355A, 358S, 360S, 364A/V, 367V, 368G/T, 369I/V/W, 370C/E/F/G/I/K/L/P/Q/R/S/V, 371L, 372A/C/F/L/R/S/V/Y, 373A/C/E/F/M/S/Y, 374E/G/L/R/S/W/Y, 375A/E/I/L/M/S/T/V, 377H, 381N, 382G/R/S/T, 384C, 386P/W, 389C/P, 391L/S, 392Y, 401L, 402G/*, 405L/Q, 406C/M/R/W, 409E/R/*, 410C/I/W/*, 411L/R/T/V, 412P/T/*, or 413A/C/D/S/*, or combinations thereof, wherein the amino acid positions are relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the amino acid sequence of the engineered protease polypeptide comprises at least a substitution at amino acid position 135, 137, 139, 141, 143, 157, 160, 214, 268, 273, 279, 311, 312, 315, 328, 342, 345, 346, or 372, or combinations thereof, wherein the amino acid positions are relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the amino acid sequence of the engineered protease polypeptide comprises at least a substitution or amino acid residue 135A/C/E/G/H/I/K/L/M/N/P/R/S/T/V/W/Y, 137A/D/N/S, 139C/D/E/F/H/I/K/L/M/N/R/S, 141A/C/D/E/F/G/H/I/L/M/N/Q/R/S/T/V/W/Y, 143A/C/D/H/N/Q/S/T, 157A/C/D/E/F/G/H/I/K/L/M/N/P/Q/R/S/T/V/W, 160A/C/D/E/F/K/L/M/N/P/R/Q/S/T/V/W/Y, 214A/C/D/E/F/G/H/I/K/L/M/N/P/Q/R/T/V/W/Y, 268A/F/G/H/I/N/P/Q/S/T/V/Y, 273A/C/F/L/M/S/T/V, 279A/E/F/G/H/K/L/M/R/S/T/Y/V/W, 311A/E/D/G/K/M/Q/S/T, 312A/C/D/E/G/H/I/K/L/M/N/P/Q/R/S/T/V/W/Y, 315A/C/D/E/F/G/H/I/K/L/M/Q/R/S/T/V/W/Y, 328L/M/V, 342A/C/D/E/F/G/I/K/M/N/P/R/Q/S/T/V/W/Y, 345A/C/D/E/F/G/I/K/L/M/Q/R/S/V/W/Y, 346A/C/D/F/G/K/L/M/N/P/Q/R/S/T/V/W/Y, or 372A/C/F/L/R/S/V/Y, or combinations thereof, wherein the amino acid positions are relative to the reference sequence corresponding to residues 135-413 of SEQ ID NO: 948, 1126, 1368, or 1548, or to the reference sequence corresponding to SEQ ID NO: 948, 1126, 1368, or 1548.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 948, or to the reference sequence corresponding to SEQ ID NO: 948, wherein the amino acid sequence comprises one or more substitutions relative to the reference sequence corresponding to residues 135-413 of SEQ ID NO: 948, or relative to the reference sequence corresponding to SEQ ID NO: 948.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 950-1154, or to a reference sequence corresponding to an even-numbered SEQ ID NO. of SEQ ID NOs: 950-1154, wherein the amino acid sequence comprises one or more substitutions relative to the reference sequence corresponding to residues 135-413 of SEQ ID NO: 948, or relative to the reference sequence corresponding to SEQ ID NO: 948.

In some embodiments, the amino acid sequence of the engineered protease polypeptide comprises at least a substitution or substitution set at amino acid position(s) 411, 402, 285, 245, 266, 355, 258, 222, 140, 268, 225, 283, 406, 410, 143/145/243/312, 139/143/145/157/312, 139/157/345, 139/269, 156/157/342/346, 139/143, 269, 139/243/328, 269/328, 143/145/169, 328, 143/145/262, 145/262/312/328, 139/156/157, 139/145/312, 312, 139, 139/312, 139/156, 139/143/145/243, 145/157, 145/346, 145/262/312/328/345/346, 145/262, 312/342, 143/243, 139/345, 342, 143/145/262/342, 139/143/169, 139/143/145/312, 169, 139/145/262/312/328/342/345/346, 139/328, 139/243, 139/143/328, 139/143/243, 139/145, 145/312, 145/169, 139/143/157/312, 84/139/143, 145/269, 143/145/157/269/312/328, 143/145/269, 157, 139/143/312, 256, 273, 409, 172, 401, 281, 253, 143/145/243/328, 145, 139/143/145/328/342/345, 143/328/342/345, 145/342/345, 143, 139/145/328/342/345, 143/145/169/312/328/345/346, 143/243/328/342/345/346, 139/143/157/169/328/346, 143/145/156/312/328, 139/145/157/312/328, 143/328/342/345/346, 143/145, 143/145/312/342/345, or 143/145/328, wherein the amino acid positions are relative to the reference sequence corresponding to residues 135-413 of SEQ ID NO: 948, or relative to the reference sequence corresponding to SEQ ID NO: 948.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 1126, or to the reference sequence corresponding to SEQ ID NO: 1126, wherein the amino acid sequence comprises one or more substitutions relative to the reference sequence corresponding to residues 135-413 of SEQ ID NO: 1126, or relative to the reference sequence corresponding to SEQ ID NO: 1126.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 1156-1422, or to a reference sequence corresponding to an even-numbered SEQ ID NO. of SEQ ID NOs: 1156-1422, wherein the amino acid sequence comprises one or more substitutions relative to the reference sequence corresponding to residues 135-413 of SEQ ID NO: 1126, or relative to the reference sequence corresponding to SEQ ID NO: 1126.

In some embodiments, the amino acid sequence of the engineered protease polypeptide comprises at least a substitution or substitution set at amino acid position(s) 279, 250, 154, 214, 249, 275, 137, 161, 180, 174, 139, 254, 145, 278, 136, 154/413, 294, 237, 274, 264, 185, 277, 293, 233, 173, 312, 302, 238, 135, 221, 290, 263, 267, 239, 163, 292, 246, 243, 235, 156, 223, 278/413, 297, 194, 251, 253/411, 145/157/253/268/273/281/312/346/411, 139/346, 253, 346/411, 253/346, 312/346, 273/312, 253/281, 157/253/273/312/346/411, 139/157/253/268/273/281/312/346, 253/273/411, 139/253/268/273/281, 139/157/411, 157, 273, 139/253/268/273/281/312/411, 157/253/411, 139/145/253/346, 139/157/253/273/312, 139/157/268/273/312/346, 157/273/312/346, 139/411, 139/253/268/273/281/312/346/411, 157/273/346/411, 139/145/157/162/253/273/281/312, 139/253/273/281/312, 157/253/268/273/281/312, 139/253/268, 139/157/312, 253/273/281/346, 157/253/312/346/411, 157/273/312/346/411, 139/145/157/253/268/281/312, 139/273/312/346, 157/253/268/273/312/346, 139/268/346, 268/273/312/346, 139/157/253/268/273/312, 139/157/253, 139/253/281, 139/157/253/268/273, 253/312/411, or 139/268/273, wherein the amino acid positions are relative to the reference sequence corresponding to residues 135-413 of SEQ ID NO: 1126, or relative to the reference sequence corresponding to SEQ ID NO: 1126.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 1368, or to the reference sequence corresponding to SEQ ID NO: 1368, wherein the amino acid sequence comprises one or more substitutions relative to the reference sequence corresponding to residues 135-413 of SEQ ID NO: 1368, or relative to the reference sequence corresponding to SEQ ID NO: 1368.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 1424-1608, or to a reference sequence corresponding to an even-numbered SEQ ID NO. of SEQ ID NOs: 1424-1608, wherein the amino acid sequence comprises one or more substitutions relative to the reference sequence corresponding to residues 135-413 of SEQ ID NO: 1368, or relative to the reference sequence corresponding to SEQ ID NO: 1368.

In some embodiments, the amino acid sequence of the engineered protease polypeptide comprises at least a substitution or substitution set at amino acid position(s) 31, 318, 296, 252, 303, 253, 413, 386, 312, 235, 412, 342, 302, 371, 405, 389, 391, 358, 139/273/311/328/372, 311, 372, 139/143/157/160/268/273/311/315, 143, 139/143, 139/160/312/372, 143/273/328, 346, 135/139/160/268/312/342/346, 139/141/273, 135/141/143/268/273/312/372, 139/141/143/311, 139/157/268/328/346/372, 53/139/141/143/273/372, 139, 139/141/143/273/312, 137/139/221/233/413, 233, 221/279, 137/139/233/279, 221, 139/214, 137/139/156, 139/214/221, 214/233, 137/139/221/233/279, 137/139/221, 137/139, 137/139/279, 137, 137/139/214/279, 266, 139/221, 137/221, 137/156/214/312, 137/221/233, 137/139/156/221, 137/139/214, 279, 137/139/233, 137/139/214/233, 221/413, 137/214/233, 137/156, 137/139/221/233, 214/221, 137/221/413, 214, 137/233, 137/413, 137/221/279, 137/139/156/214/233/413, or 137/139/156/214, wherein the amino acid positions are relative to the reference sequence corresponding to residues 135-413 of SEQ ID NO: 1368, or relative to the reference sequence corresponding to SEQ ID NO: 1368.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 1548, or to the reference sequence corresponding to SEQ ID NO: 1548, wherein the amino acid sequence comprises one or more substitutions relative to the reference sequence corresponding to residues 135-413 of SEQ ID NO: 1548, or relative to the reference sequence corresponding to SEQ ID NO: 1548.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 1610-2242, or to a reference sequence corresponding to an even-numbered SEQ ID NO. of SEQ ID NOs: 1610-2242, wherein the amino acid sequence comprises one or more substitutions relative to the reference sequence corresponding to residues 135-413 of SEQ ID NO: 1548, or relative to the reference sequence corresponding to SEQ ID NO: 1548.

In some embodiments, the amino acid sequence of the engineered protease polypeptide comprises at least a substitution or substitution set at amino acid position(s) 145/273/372, 145/256/273, 221/243/273/328/372, 372, 145/221/279/372/406, 273/328, 145/169/273/346/406, 256/273, 145/221/273/328/346/406, 243/273, 145/214/256/273/279/328/372, 169/221/328/372/406, 372/406, 169/328/372/406, 221/372, 145/221/273/328/372, 214/243/273/328, 145/221, 214/256/273/346/372, 221/406, 169/372, 145/214/221/273, 145/221/346/372, 243/273/328/372/406, 145/169/273/328/346, 328, 169/273/372, 145/221/328, 169/214/273, 221/273/328, 221, 145/328, 214/346, 312, 212, 279, 212/312, 145, 179/346, 214, 346, 315/372, 375, 264, 179, 185, 220/372, or 324, wherein the amino acid positions are relative to the reference sequence corresponding to residues 135-413 of SEQ ID NO: 1548, or relative to the reference sequence corresponding to SEQ ID NO: 1548.

In some embodiments, the amino acid sequence of the engineered protease polypeptide comprises at least a substitution set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the amino acid sequence of the engineered protease polypeptide comprises at least a substitution or substitution set of an engineered protease polypeptide variant set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to an amino acid sequence comprising at least a substitution or substitution set of an engineered protease polypeptide variant set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the engineered protease polypeptide comprises an amino acid sequence comprising residues 135-413 of an even-numbered SEQ ID NO. of SEQ ID NOs: 6-2242, or an amino acid sequence comprising an even-numbered SEQ ID NO. of SEQ ID NOs: 6-2242.

In some embodiments, the engineered protease polypeptide comprises an amino acid sequence comprising residues 135-413 of SEQ ID NO: 628, 948, 1126, 1368, 1548, 1640, or 1710, or an amino acid sequence comprising SEQ ID NO: 628, 948, 1126, 1368, 1548, 1640, or 1710.

In some embodiments, the engineered protease polypeptide is capable of converting to a proteolytically active polypeptide or is an active protease. In some embodiments, the amino acid sequence of the engineered protease polypeptide comprises amino acid residues 135-413 or amino acid residues 128-413, wherein the engineered protease polypeptide is proteolytically active or is an active protease.

In some embodiments, the proteolytic active polypeptide or active protease of an engineered protease polypeptide is characterized by an improved property selected from: i) increased protease activity, ii) increased resistance to pepsin, iii) increased stability and/or activity at acidic pH, iv) increased stability and/or activity at neutral pH, or v) increased thermostability, or any combination of i), ii), iii), iv), and v) as compared to a reference protease. In some embodiments, the reference protease has an amino acid sequence corresponding to residues 135-413 of SEQ ID NO: 4, 628, 948, 1126, 1368, or 1548, or an amino acid sequence corresponding to SEQ ID NO: 4, 628, 948, 1126, 1368, or 1548. In some embodiments, the reference protease has an amino acid sequence corresponding to residues 135-413 of SEQ ID NO: 4 or 628, or an amino acid sequence corresponding to SEQ ID NO: 4, 628.

In some embodiments, the engineered protease polypeptide comprises at least a carboxy terminal deletion of SEQ ID NO: 2, wherein the deletion maintains protease activity of the mature form of SEQ ID NO: 2 with the carboxy terminal deletion. In some embodiments, the carboxy terminal deletion comprise deletion of the Big1 domain. In some embodiments, the carboxy terminal deletion is up to and including amino acid residue 426, or up to and including amino acid residue 414 of SEQ ID NO: 2. In some embodiments, the engineered protease polypeptide further comprises 1, 2, 3, 4, 5, 6, 7, 8, 9, or up to 10 amino acid deletions of the carboxy terminus at amino acid residue 413 of SEQ ID NO: 2, wherein the further amino acid deletion(s) maintains proteolytic activity of the mature form of SEQ ID NO: 2 having the further amino acid deletions. In some embodiments, the engineered protease polypeptide is the mature form having an amino terminus at amino acid residue 128 or 135 of SEQ ID NO: 2.

In another aspect, the present disclosure provides a recombinant polynucleotide comprising a polynucleotide sequence encoding an engineered protease polypeptide described herein.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference polynucleotide sequence corresponding to nucleotide residues 403 to 1239 of SEQ ID NO: 3, 627, 947, 1125, 1367, or 1547, or to a reference polynucleotide corresponding to SEQ ID NO: 3, 627, 947, 1125, 1367, or 1547, wherein the recombinant polynucleotide encodes an engineered protease polypeptide.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference polynucleotide sequence corresponding to nucleotide residues 403 to 1239 of an odd-numbered SEQ ID NO. of SEQ ID NOs: 5-2241, or to a reference polynucleotide corresponding to an odd-numbered SEQ ID NO. of SEQ ID NOs: 5-2241, wherein the recombinant polynucleotide encodes an engineered protease polypeptide.

In some embodiments, the polynucleotide sequence of the recombinant polynucleotide encoding an engineered protease polypeptide is codon optimized. In some embodiments, the polynucleotide sequence is codon optimized for expression in a bacterial cell, fungal cell, insect cell, or mammalian cell.

In some embodiments, the recombinant polynucleotide comprises a polynucleotide sequence comprising nucleotide residues 403-1239 of an odd-numbered SEQ ID NO. of SEQ ID NOs: 5-1709, or comprising an odd-numbered SEQ ID NO. of SEQ ID NOs: 5-1709.

In another aspect, the present disclosure further provides an expression vector comprising a recombinant polynucleotide encoding an engineered protease polypeptide described herein. In some embodiments, the expression vector further comprises a control sequence operably linked to the recombinant polynucleotide. In some embodiments, the control sequence comprises at least a promoter, particularly a heterologous promoter.

In another aspect, the present disclosure provides a host cell comprising an expression vector comprising a recombinant polynucleotide encoding an engineered protease polypeptide. In some embodiments, the host cell is a bacterial cell, fungal cell, insect cell, or mammalian cell.

In a further aspect, also provided is a method of producing an engineered protease polypeptide, comprising culturing a host cell described herein under suitable conditions such that the encoded engineered protease is expressed or produced. In some embodiments, the method further comprises isolating the expressed or produced engineered protease polypeptide from culture medium and/or cells.

In some embodiments, the method further comprises purifying the expressed or produced engineered protease polypeptide.

In some embodiments, provided herein is a method of preparing a proteolytically active protease polypeptide, comprising incubating an engineered protease polypeptide described herein under suitable conditions such that the proteolytically active protease polypeptide or active protease is produced. In some embodiments, the proteolytically active protease polypeptide or active protease has an amino terminus at amino acid residue 128 or 135, wherein the amino acid positions are numbered with respect to SEQ ID NO: 4, or equivalent positions thereof for any engineered protease polypeptide variant.

In some embodiments, the suitable conditions for preparing a proteolytically active protease polypeptide is sufficient for activation of the engineered protease polypeptide. In some embodiments, the method for preparing a proteolytically active protease comprises incubating the engineered protease polypeptide under suitable conditions for autoproteolysis. In some embodiments, the method for preparing a proteolytically active protease comprises contacting the engineered protease polypeptide with a proteolytically active polypeptide or an active protease of an engineered protease polypeptide described herein.

In another aspect, the engineered protease polypeptide is provided as a composition. In some embodiments, the composition comprises an engineered protease polypeptide and a protein-containing food or drink. In some embodiments, the composition comprises an engineered protease polypeptide admixed with a protein-containing food or drink.

In some embodiments, the engineered protease polypeptide is provided as a pharmaceutical composition. In some embodiments, the pharmaceutical composition further comprises a pharmaceutically acceptable excipient and/or carrier. In some embodiments, the pharmaceutical composition comprises an effective amount of the engineered protease for treating exocrine pancreatic insufficiency.

In some embodiments, the engineered protease polypeptide is used in the treatment of a condition or disease associated with a deficiency in pancreatic digestive enzymes. In some embodiments, a method of treating a disease or condition associated with a deficiency in pancreatic enzymes, comprises administering to a subject in need thereof an effective amount of an engineered protease polypeptide described herein or a pharmaceutical composition thereof. In some embodiments, the disease or condition associated with a deficiency in pancreatic digestive enzymes is exocrine pancreatic insufficiency.

In some embodiments, the engineered protease polypeptide or pharmaceutical composition thereof is administered immediately prior to, concurrently with, or subsequent to consumption of a protein-containing food or drink.

In some embodiments, the subject for treatment with an engineered protease polypeptide is a human infant or child. In some embodiments, the subject for treatment with an engineered protease polypeptide is a human adult.

In some embodiments, an engineered protease polypeptide is used for treating exocrine pancreatic insufficiency.

In some embodiments, an engineered protease polypeptide is used in the preparation of a medicament for treating exocrine pancreatic insufficiency.

### BRIEF DESCRIPTION OF THE DRAWINGS

FIG. 1 shows the full length naturally occurring amino acid sequence of the protease pro-polypeptide from Bacillus sinesaloumensis Marseille P3516 with a His-tag sequence (italicized) at the carboxy terminus. The polypeptide is composed of the pro-domain, which is within residues 1 to 127; a protease domain, which is within residues 128 to 413; and a bacterial Ig-like domain 1 (Big1-1), which is within residues 440 to 522.

FIG. 2 shows the naturally occurring amino acid sequence of the pro-polypeptide of the protease from Bacillus sinesaloumensis Marseille P3516 (SEQ ID NO: 4). The pro-polypeptide is converted to an active protease by cleavage between amino acid residues 127/128 and/or 134/135 in the illustrated sequence.

### DETAILED DESCRIPTION

The present disclosure provides engineered protease polypeptide, a proteolytically active engineered protease polypeptide, recombinant polynucleotides encoding the engineered protease polypeptides, and use of the engineered protease polypeptides. In some embodiments, the protease polypeptide is engineered to have advantageous properties compared to the naturally occurring protease, including among others, enhanced expression, increased proteolytic activity of the active protease, increased thermostability, increased resistance against other proteases, increased activity at acidic pH, and increased stability at acidic pH. In some embodiments, the enhanced properties of the engineered protease make is useful as a therapeutic in the treatment of exocrine pancreatic insufficiency, e.g., as enzyme replacement therapy (ERT).

### Abbreviations and Definitions

In reference to the present disclosure, the technical and scientific terms used in the descriptions herein will have the meanings commonly understood by one of ordinary skill in the art, unless specifically defined otherwise.

Furthermore, the headings provided herein are not limitations of the various aspects or embodiments of the invention which can be had by reference to the application as a whole.

It is to be understood that the invention herein is not limited to the particular methodology, protocols, and reagents described, as these may vary, depending upon the context they are used by those of skill in the art. Accordingly, the terms defined immediately below are more fully described by reference to the application as a whole.

As used herein, the singular “a”, “an,” and “the” include the plural references, unless the context clearly indicates otherwise.

As used herein, the term “comprising” and its cognates are used in their inclusive sense (i.e., equivalent to the term “including” and its corresponding cognates).

It is to be further understood that where description of embodiments use the term “comprising” and its cognates, the embodiments can also be described using language “consisting essentially of” or “consisting of.”

Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

“About” as used herein means an acceptable error for a particular value. In some instances, “about” means within 0.05%, 0.5%, 1.0%, or 2.0%, of a given value range. In some instances, “about” means within 1, 2, 3, or 4 standard deviations of a given value.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Polynucleotide” is used herein to denote a polymer comprising at least two nucleotides where the nucleotides are either deoxyribonucleotides or ribonucleotides. The abbreviations used for the genetically encoding nucleosides are conventional and are as follows: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated.

“Protein,” “polypeptide,” and “peptide” are used interchangeably to denote a polymer of at least two amino acids covalently linked by an amide bond, regardless of length or post-translational modification (e.g., glycosylation or phosphorylation). Unless indicated otherwise, amino acid sequences are written left to right in amino to carboxy orientation.

“Amino acids” are referred to herein by either their commonly known three-letter symbols or by the one-letter symbols recommended by IUPAC-IUB Biochemical Nomenclature Commission. The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (Cα). For example, whereas “Ala” designates alanine without specifying the configuration about the α carbon, “D-Ala” and “L-Ala” designate D-alanine and L-alanine, respectively. When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

“Fusion protein” or “fusion polypeptide” refer to hybrid proteins created through the joining of two or more genes or polynucleotides that originally encoded separate proteins. In some embodiments, fusion proteins and fusion polypeptides are created by recombinant technology (e.g., molecular biology techniques known in the art).

“Protease,” “proteinase,” and “peptidase” refer to enzymes that hydrolyze proteins, polypeptides, and/or oligopeptides. Proteases, proteinases, and peptidases breakdown proteins, polypeptides, or oligonucleotides into smaller peptides or single amino acids.

“Proteolysis” or “proteolytic activity” refers to the breakdown (e.g., through hydrolysis) of proteins and/or polypeptides into smaller peptides and/or amino acids.

“Auto-proteolysis” refers to the self breakdown of the subject protein, for example the breakdown of proteases through their own action on their structures.

“Lipase” refers to any enzyme commonly referred to as “lipase” that catalyzes the hydrolysis of fats by hydrolyzing the ester bonds of triglycerides. Pancreatic lipases are important in the breakdown of fats to fatty acids, glycerol, and other alcohols. Lipases are essential in the digestion, transport, and processing of dietary lipids in most organisms.

“Lipid” refers to a class of water-insoluble macromolecules that include fatty acids and their esters, sterols, prenols, certain poorly soluble vitamins, and other related compounds. “Fats” are a subset of lipids composed of fatty acid esters (e.g., triglycerides, which are made from glycerol and three fatty acids). It is not intended that the present invention be limited to any specific lipid and/or fat. Taking the context into consideration, the terms “fat” and “lipid” are used interchangeably herein.

“Amylase” refers to an enzyme that is capable of hydrolyzing glycosidic bonds in starch to converting it to smaller polysaccharides, such as disaccharides (e.g., maltose) and trisaccharides, or simple sugars, such as glucose.

“Mature protein” or “mature polypeptide” refers to the final processed biological protein or polypeptide or product.

“Pro-protein,” “pro-polypeptide,” or “pro-peptide” refers to a precursor protein, polypeptide, or peptide that is processed by post-translational modification, to form a biologically active protein, polypeptide, or peptide. In some embodiments, the post translational modification is a cleavage reaction to form the protein, polypeptide, or peptide. “Pro-enzyme” refers to a precursor polypeptide that is processed by post-translational modification, in particular a cleavage reaction, to form an active enzyme.

“Pre-pro-protein,” “pre-pro-polypeptide,” or “pre-pro-peptide” refers to a precursor protein, polypeptide, or peptide that includes a signal sequence and which can be processed by posttranslational modification, in particular a cleavage reaction, to generate a pro-protein, pro-polypeptide, or pro-peptide. Generally, a cleavage reaction removes a signal sequence to generate a pro-protein, pro-polypeptide, or pro-peptide. “Pre-pro-enzyme” refers to a precursor protein, polypeptide, or peptide that is processed by post-translational modification, in particular a cleavage reaction that removes a signal sequence, to form a pro-enzyme.

“Full-length” in context of a protein or polypeptide refers to the protein or polypeptide which is not processed to alter the amino acid sequence of the entire protein or polypeptide. For example, a full-length protein is the entire protein encoded in the corresponding mRNA.

“Engineered,” “recombinant,” “non-naturally occurring,” and “variant,” when used with reference to a cell, a polynucleotide or a polypeptide refers to a material or a material corresponding to the natural or native form of the material that has been modified in a manner that would not otherwise exist in nature or is identical thereto but produced or derived from synthetic materials and/or by manipulation using recombinant techniques.

“Wild-type” and “naturally-occurring” refer to the form found in nature. For example, a wild-type polypeptide or polynucleotide sequence is a sequence present in an organism that can be isolated from a source in nature and which has not been intentionally modified by human manipulation.

“Coding sequence” refers to that part of a nucleic acid (e.g., a gene) that encodes an amino acid sequence of a protein.

“Percent (%) sequence identity” is used herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted (e.g., by the local homology algorithm of Smith and Waterman; Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA., 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms (see, e.g., Altschul et al., J. Mol. Biol., 1990, 215:403-410; and Altschul et al., Nucleic Acids Res., 1977, 25:3389-3402). Software for performing BLAST analyses is publicly available through the National Center for Biotechnology Information website. This algorithm involves first identifying high scoring sequence pairs (HSPs) by identifying short words of length “W” in the query sequence, which either match or satisfy some positive-valued threshold score “T,” when aligned with a word of the same length in a database sequence. T is referred to as the neighborhood word score threshold (See, Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (reward score for a pair of matching residues; always >0) and “N” (penalty score for mismatching residues; always <0). For amino acid sequences, a scoring matrix is used to calculate the cumulative score. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see, e.g., Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full-length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence.

“Comparison window” refers to a conceptual segment of contiguous nucleotide positions or amino acids residues wherein a sequence may be compared to a reference sequence. In some embodiments, the comparison window is at least 15 to 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. In some embodiments, the comparison window can be longer than 15-20 contiguous residues, and includes, optionally 30, 40, 50, 100, or longer windows.

“Corresponding to,” “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered protease polypeptide, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Mutation” refers to any change in a polypeptide or polynucleotide sequence. It is intended to encompass any number (i.e., one or more) of substitutions, insertions, deletions, and/or rearrangements present in a sequence (i.e., as compared to the starting or reference sequence). Thus, mutations in polynucleotide sequences can result in the production of variant polypeptides (e.g., variant or engineered proteases), as provided herein. In some embodiments, where the reference sequence is a subsequence of a longer sequence, amino acid positions and corresponding mutations (e.g., substitutions) or mutation sets within the subsequence are selected.

“Amino acid difference” and “residue difference” refer to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X135 as compared to SEQ ID NO: 4” (or a “residue difference at position 135 as compared to SEQ ID NO: 4”) refers to a difference of the amino acid residue at the polypeptide position corresponding to position 135 of SEQ ID NO: 4. Thus, if the reference polypeptide of SEQ ID NO: 4 has an alanine at position 135, then a “residue difference at position X135 as compared to SEQ ID NO: 4” refers to an amino acid substitution with any residue other than alanine at the position of the polypeptide corresponding to position 135 of SEQ ID NO: 4. In some instances herein, the specific amino acid residue difference at a position is indicated as “XnY” where “Xn” specifies the corresponding residue and position of the reference polypeptide (as described above), and “Y” is the single letter identifier of the amino acid found in the engineered or recombinant polypeptide (i.e., the different residue than in the reference polypeptide). In some embodiments, the amino acid difference, e.g., a substitution, is denoted by the abbreviation “nY,” without the identifier for the residue in the reference sequence. In some embodiments, the phrase “an amino acid residue nY” denotes the presence of the amino residue in the engineered or recombinant polypeptide, which may or may not be a substitution in context of a reference sequence. In some embodiments, the “substitution” comprises the deletion of an amino acid, which can be denoted by “−”, or a replacement with a termination codon, which can be denoted by “*”.

In some instances, a polypeptide of the present disclosure can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X151D/X151Q, X151D/Q, or 151D/Q).

“Amino acid substitution set” and “substitution set” refers to a group of amino acid substitutions within a polypeptide sequence. In some embodiments, substitution sets comprise 2, 3, 4, 5, 6, 7, 8, 9, 10, 11, 12, 13, 14, 15, or more amino acid substitutions. In some embodiments, a substitution set refers to the set of amino acid substitutions that is present in any of the variant protease polypeptides listed in any of the Tables in the Examples. In these substitution sets, the individual substitutions are separated by a semicolon (“;”; e.g., A126T;G192C) or slash (“/”; e.g., A126T/G192C or 126T/192C). In some embodiments, the phrase “mutation set” can be used.

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the properties of a protease polypeptide. Deletions can be directed to the internal portions and/or terminal portions of the polypeptide. In various embodiments, the deletion can comprise a continuous segment or can be discontinuous.

“Insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Functional fragment” and “biologically active fragment” are used interchangeably herein, to refer to a polypeptide that has an amino-terminal and/or carboxy-terminal deletion(s) and/or internal deletions, but where the remaining amino acid sequence is identical to the corresponding positions in the sequence to which it is being compared (e.g., a full-length engineered protease polypeptide) and that retains substantially all of the activity of the full-length polypeptide.

“Isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The engineered protease polypeptides may be present within a cell, present in the cellular medium, or prepared in various forms, such as lysates or isolated preparations. As such, in some embodiments, the engineered protease polypeptides provided herein are isolated polypeptides.

“Substantially pure polypeptide” or “purified polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure protease polypeptide composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated engineered protease polypeptides are substantially pure polypeptide compositions.

“Improved enzyme property” and “improved property” refers to a property of an engineered protease polypeptide which comprises an improvement in any enzyme property as compared to a reference protease polypeptide and/or as a wild-type protease polypeptide or another engineered protease polypeptide. Improved properties include but are not limited to such properties as increased protein expression, increased thermostability, increased pH activity, increased stability, increased enzymatic activity, increased substrate specificity or affinity, increased chemical stability, improved solvent stability, increased tolerance to acidic or basic pH, increased tolerance to protease activity (i.e., reduced sensitivity to proteolysis), reduced aggregation, increased solubility, and altered temperature profile.

“Increased enzymatic activity” or “enhanced catalytic activity” refers to an improved property of the engineered protease polypeptides, that can be represented by an increase in specific activity (e.g., product produced/time/weight protein) or an increase in percent conversion of the substrate to the product (e.g., percent conversion of starting amount of substrate to product in a specified time period using a specified amount of protease) as compared to the reference protease enzyme. Exemplary methods to determine enzyme activity are provided in the Examples. Any property relating to enzyme activity may be affected, including the classical enzyme properties of Kₘ, Vₘₐₓ or k_{cat}, changes of which can lead to increased enzymatic activity. Improvements in enzyme activity can be from about 1.1 fold the enzymatic activity of the corresponding wild-type enzyme, to as much as 2-fold, 5-fold, 10-fold, 20-fold, 25-fold, 50-fold, 75-fold, 100-fold, 150-fold, 200-fold or more enzymatic activity than the naturally occurring protease or another engineered protease from which the protease polypeptides were derived.

“Protease stable” and “stability to proteolysis” refer to the ability of a protein (e.g., an engineered protease of the present invention) to function and withstand proteolysis mediated by any proteolytic enzyme or other proteolytic compound or factor and retain its function following treatment with the protease. It is not intended that the term be limited to the use of any particular protease to assess the stability of a protein. In some embodiments, the engineered proteases are stable in the presence of a gastric protease.

“pH stability” refers to the ability of a protein (e.g., an engineered protease of the present invention) to function after incubation at a particular pH. In some embodiments, the present disclosure provides engineered proteases that are stable at a range of pHs, including, acid, neutral, and/or basic pH. In some embodiments, the engineered proteases are stable at different pH ranges, as indicated in the Examples provided herein.

“Physiological pH” refers to the pH range generally found in a subject's (e.g., human) blood (e.g., pH 7.2-7.4).

“Basic pH” (e.g., used with reference to improved stability to basic pH conditions or increased tolerance to basic pH) means a pH range of >7, for example >pH 7 to 11, or in some embodiments, greater than pH 11.

“Acidic pH” (e.g., used with reference to improved stability to acidic pH conditions or increased tolerance to acidic pH) means a pH range that encompasses any pH values <7. In some embodiments, the acid pH is less than 7, while in some other embodiments, the pH is less than about 6, 5, 4, 3, 2, or lower. In some alternative embodiments, the engineered proteases of the present disclosure are stable at pH levels of 2 to 4.

“Improved tolerance to acidic pH” means that an engineered protease according to the invention will have increased stability (higher retained activity at <pH 7, e.g., 6, 5, 4 3, 2, or even lower, after exposure to the acidic pH for a specified period of time (e.g., 1 hour, up to 24 hours, etc.) as compared to a reference protease or another enzyme.

“Improved tolerance to basic pH” means that an engineered protease according to the invention will have increased stability (higher retained activity at about pH >7, e.g., 8, or 9, or even higher, after exposure to basic pH for a specified period of time, e.g., 1 hour, up to 24 hours, etc., as compared to a reference protease or another enzyme.

“Gastric challenge” refers to the exposure of the engineered proteases of the present invention to a low pH environment and the presence of at least one gastric enzyme, such as a protease (e.g., pepsin), such that the recombinant protease is exposed to the conditions that may be encountered in the stomach (e.g., the human stomach).

“Thermal stability” and “thermostability” refer to the ability of a protein (e.g., an engineered protease of the present invention) to function at a particular temperature. In some embodiments, the term refers to the ability of a protein to function following incubation at a particular temperature. In some embodiments, the engineered proteases of the present invention are “thermotolerant” (i.e., the enzymes maintain their catalytic activity at elevated temperatures). In some embodiments, the engineered proteases resist inactivation at elevated temperatures and in some embodiments, maintain catalytic activity at elevated temperatures for prolonged exposure times. In some embodiments, thermal stability is measured following incubation of a protein (e.g., an engineered protease of the present invention) at a particular temperature.

“Suitable reaction conditions” refers to those conditions in the enzymatic conversion reaction solution (e.g., ranges of enzyme loading, substrate loading, temperature, pH, buffers, co-solvents, etc.) under which a protease polypeptide of the present application is capable of converting a substrate to the desired product compound. Exemplary “suitable reaction conditions” are provided in the present application and illustrated by the Examples.

“Codon optimized” refers to changes in the codons of the polynucleotide encoding a protein to those preferentially used in a particular organism such that the encoded protein is more efficiently expressed in that organism. Although the genetic code is degenerate, in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the protease polypeptide are codon optimized for optimal production from the host organism selected for expression.

“Control sequence” refers herein to include all components that are necessary or advantageous for the expression of a polynucleotide and/or polypeptide of the present disclosure. Each control sequence may be native or foreign to the nucleic acid sequence encoding the polypeptide. Such control sequences include, but are not limited to, leaders, polyadenylation sequences, pro-peptide sequences, promoter sequences, signal peptide sequences, initiation sequences, and transcription terminators. In some embodiments, at a minimum, the control sequences include a promoter, and transcriptional and translational stop signals.

“Operably linked” is defined herein as a configuration in which a control sequence is appropriately placed (i.e., in a functional relationship) at a position relative to a polynucleotide of interest such that the control sequence directs or regulates the expression of the polynucleotide and/or encoded polypeptide of interest.

“Heterologous” or “recombinant” refers to the relationship between two or more nucleic acid or polypeptide sequences (e.g., a promoter sequence, signal peptide, terminator sequence, etc.) that are derived from different sources and are not associated in nature.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Vector” refers to a polynucleotide construct for introducing a polynucleotide sequence into a cell. In some embodiments, the vector is an expression vector that is operably linked to a suitable control sequence capable of effecting the expression in a suitable host of the polypeptide encoded in the polynucleotide sequence. In some embodiments, an “expression vector” has a promoter sequence operably linked to the polynucleotide sequence (e.g., transgene) to drive expression in a host cell, and in some embodiments, also comprises a transcription terminator sequence.

“Culturing” refers to the growing of a population of cells, such as host cells, under suitable conditions using any suitable medium (e.g., liquid, gel, or solid). In some embodiments, the cells are microbial cells (e.g., bacteria), while in some other embodiments, the cells are mammalian cells, insect cells, or cells obtained from another animal. It is not intended that the present invention be limited to culturing of any particular cells or cell types or any specific method of culturing.

“Expression” includes any step involved in the expression of a polypeptide including, but not limited to, transcription, post-transcriptional modification, translation, and post-translational modification. In some embodiments, the term also encompasses secretion of the polypeptide from a cell. “Produces” refers to the expression of proteins and/or other compounds by cells.

“Host cell” and “host strain” refer to suitable hosts for expression vectors comprising polynucleotides provided herein (e.g., a polynucleotide sequences encoding at least one protease polypeptide). In some embodiments, the host cells are prokaryotic or eukaryotic cells that have been transformed or transfected with vectors constructed using recombinant techniques as known in the art.

“Hybridization stringency” relates to hybridization conditions, such as washing conditions, in the hybridization of nucleic acids. Generally, hybridization reactions are performed under conditions of lower stringency, followed by washes of varying but higher stringency. The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tm as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Another high stringency condition is hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited above.

“Composition” and “formulation” encompass products comprising at least one engineered protease of the present invention, intended for any suitable use (e.g., pharmaceutical compositions, dietary and/or nutritional supplements, etc.).

“Pharmaceutical composition” refers to a composition suitable for pharmaceutical use in a subject (e.g., human).

“Pharmaceutically acceptable” means a material that can be administered to a subject without causing any undesirable biological effects or interacting in a deleterious manner with any of the components in which it is contained and that possesses the desired biological activity.

“Excipient” refers to any pharmaceutically acceptable additive, carrier, diluent, adjuvant, or other ingredient, other than the active pharmaceutical ingredient. Excipients are typically included for formulation and/or administration purposes.

“Carrier” when used in reference to a pharmaceutical composition means any of the standard pharmaceutical carrier, buffers, and excipients, such as stabilizers, preservatives, and adjuvants.

“Administration” and “administering” a composition mean providing a composition of the present invention to a subject, such as a patient.

“Concurrent administration,” or “co-treatment,” as used herein includes administration of the agents together, or before or after each other.

“Effective amount” means an amount sufficient to produce the desired result. One of general skill in the art may determine what the effective amount by using experimentation.

“Therapeutically effective amount” when used in reference to symptoms of disease/condition refers to the amount and/or concentration of a compound (e.g., engineered protease polypeptides) that ameliorates, attenuates, or eliminates one or more symptom of a disease/condition or prevents or delays the onset of symptom(s). A “therapeutically effective amount” when used in reference to a disease/condition refers to the amount and/or concentration of a composition (e.g., engineered protease polypeptides) that ameliorates, attenuates, or eliminates the disease/condition. In some embodiments, the term is used in reference to the amount of a composition that elicits the biological (e.g., medical) response by a tissue, system, or animal subject that is sought by the researcher, physician, veterinarian, or other clinician.

“Treating” or “treatment” of a disease, disorder, or syndrome, as used herein, includes (i) preventing the disease, disorder, or syndrome from occurring in a subject, i.e., causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As such, the terms “treating,” “treat” and “treatment” encompass preventative (e.g., prophylactic), as well as palliative treatment. As is known in the art, adjustments for systemic versus localized delivery, age, body weight, general health, sex, diet, time of administration, drug interaction and the severity of the condition may be necessary, and will be ascertainable by one of ordinary skill in the art.

“Modulate,” “attenuate” or “ameliorate” means any treatment of a disease or disorder in a subject, such as a mammal, including: preventing or protecting against the disease or disorder, e.g., causing the abnormal biological reaction or symptoms not to develop; inhibiting the disease or disorder, arresting or suppressing the development of abnormal biological reactions and/or clinical symptoms; and/or relieving the disease or disorder, e.g., causing the regression of abnormal biological reactions and/or symptoms.

“Preventing” or “inhibiting” refers to the prophylactic treatment of a subject in need thereof. The prophylactic treatment can be accomplished by providing an appropriate dose of a therapeutic agent to a subject at risk of suffering from an ailment, thereby substantially averting onset of the ailment.

“Subject” encompasses mammals such as humans, non-human primates, livestock, companion animals, and laboratory animals (e.g., rodents and lagamorphs). It is intended that the term encompass females as well as males. In some embodiments, a “patient” means any subject that is being assessed for, treated for, or is experiencing disease.

“Infant” refers to a child in the period of the first month after birth to approximately one (1) year of age. As used herein, the term “newborn” refers to child in the period from birth to the 28th day of life.

“Child” refers to a person who has not attained the legal age for consent to treatment or research procedures. In some embodiments, the term refers to a person between the time of birth and adolescence.

In some embodiments, “child” can be further subdivided into children older than 12 months and younger than 4 years, and children 4 years and older up to 18 years of age.

“Adult” refers to a person who has attained legal age for the relevant jurisdiction (e.g., 18 years of age in the United States). In some embodiments, the term refers to any fully grown, mature organism.

### Engineered Protease Polypeptides

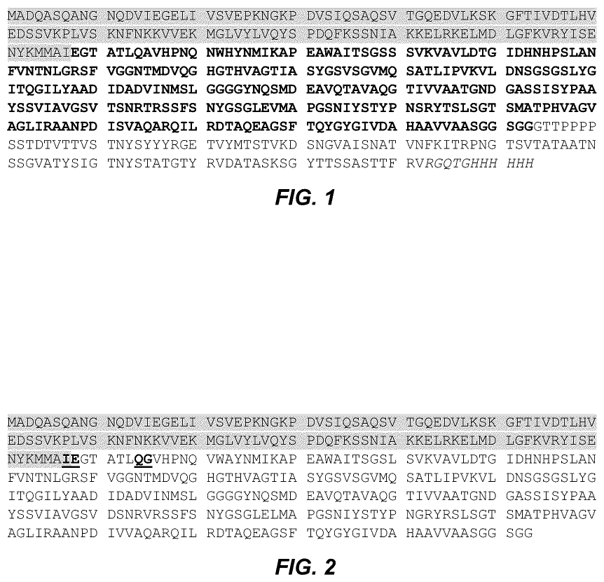

The engineered protease polypeptides described herein is based on the naturally occurring protease of Bacillus sinesaloumensis Marseille P3516. The naturally occurring protease is composed of a pro-region, a protease domain, and a Big-1 domain (see FIG. 1 and SEQ ID NO: 2). The Big-1 domain is a region of about 95 amino acids present in bacterial adhesion molecules of the intimin/invasin family. In the amino acid sequence of SEQ ID NO: 2, the Big-1 domain is within amino acid residues about 440 to 522. The present disclosure shows that protease activity is maintained in engineered proteases deleted of the Big-1 domain, as well as deletion of a peptide region linking the Big-1 domain to the protease domain. Deletion of the carboxy terminal region up to and including amino acid residue 426, or up to and including amino acid residue 414 maintains the protease activity.

Furthermore, the pro-polypeptide or pro-enzyme form of the naturally occurring protease can transform into or covert to an active protease. Without being bound by any theory of operation, the pro-domain appears to promote formation of the active protease, which is formed by cleavage of the pro-polypeptide. For the pro-polypeptide of SEQ ID NO: 4 (see FIG. 2), the cleavage can occur between amino acid residues 127/128 and/or 134/135 to form the proteolytically active polypeptide or active protease. This cleavage can occur through auto-proteolysis, a self catalyzed reaction that occurs under suitable activation conditions. In some instances, the proteolytically active polypeptide may be formed by action of another protease, including the active protease forms of the engineered protease polypeptide described herein.

In one aspect, the present disclosure provides engineered protease polypeptides, with or without the Big-1 domain, as well as engineered protease polypeptides having one or more amino acid deletions of the peptide region linking the Big-1 domain to the protease domain. In some embodiments, the engineered protease polypeptides include the pro-polypeptide and corresponding proteolytically active polypeptide form, e.g., an active protease or the mature form of the protease.

In some embodiments, the present disclosure provides engineered protease polypeptides which exhibit an improved property, including, among others, enhanced expression, increased proteolytic activity of the active protease, increased thermostability, increased resistance against gastric proteases, increased activity at acidic pH, and/or increased stability at acidic pH.

In some embodiments, an engineered protease polypeptide, or a biologically active fragment thereof, comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of SEQ ID NO: 4, 628, 948, 1126, 1368, or 1548, or to a reference sequence corresponding to SEQ ID NO: 4, 628, 948, 1126, 1368, or 1548, wherein the amino acid sequence comprises one or more substitutions relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the engineered protease polypeptide, or a biologically active fragment thereof, comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 4 or 628, or to the reference sequence corresponding to SEQ ID NO: 4 or 628, wherein the amino acid sequence comprises one or more substitutions relative to the reference sequence corresponding to residues 135-413 of SEQ ID NO: 4 or 628, or relative to the reference sequence corresponding to SEQ ID NO: 4 or 628.

In some embodiments, the engineered protease polypeptide, or a biologically active fragment thereof, comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 948, 1126, 1368, or 1548, or to the reference sequence corresponding to SEQ ID NO: 948, 1126, 1368, or 1548, wherein the amino acid sequence comprises one or more substitutions relative to the reference sequence corresponding to residues 135-413 of SEQ ID NO: 4 or 628, or relative to the reference sequence corresponding to SEQ ID NO: 4 or 628.

In some embodiments, the engineered protease polypeptide, or a biologically active fragment thereof, of comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 6-2242, or to a reference sequence corresponding to an even-numbered SEQ ID NO. of SEQ ID NOs: 6-2242, wherein the amino acid sequence comprises one or more substitutions relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution at amino acid position 11, 31, 42, 45, 50, 53, 84, 99, 100, 126, 128, 129, 130, 131, 132, 134, 135, 136, 137, 138, 139, 140, 141, 143, 145, 151, 154, 156, 157, 159, 160, 161, 162, 163, 169, 172, 173, 174, 179, 180, 184, 185, 186, 187, 188, 190, 191, 192, 193, 194, 198, 199, 212, 214, 220, 221, 222, 223, 225, 231, 232, 233, 235, 237, 238, 239, 240, 242, 243, 245, 246, 249, 250, 251, 252, 253, 254, 256, 258, 262, 263, 264, 265, 266, 267, 268, 269, 271, 273, 274, 275, 277, 278, 279, 280, 281, 283, 285, 290, 292, 293, 294, 296, 297, 300, 302, 303, 311, 312, 313, 314, 315, 316, 318, 324, 328, 336, 339, 341, 342, 343, 345, 346, 355, 358, 360, 364, 367, 368, 369, 370, 371, 372, 373, 374, 375, 377, 381, 382, 384, 386, 389, 391, 392, 401, 402, 405, 406, 409, 410, 411, 412, or 413, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or amino acid residue 11K, 31G, 42W, 45Y, 50R, 53A, 84M, 99V, 100V, 126T, 128G/I/K/L/P/R/S/T/V, 129E/F/H/I/K/L/R/S/T/V, 130A/F/G/N/V, 131E/P/R/T/V/Y, 132A/C/D/E/G/P/R/V/Y, 134A/C/D/E/G/I/L/M/N/P/S/T/V/W/Y, 135C/E/G/H/I/K/L/M/N/P/R/S/T/V/W/Y, 136C/G/I/M, 137A/D/N/S, 138Q, 139C/D/E/F/H/I/K/L/M/R/S, 140L, 141A/C/D/E/F/G/H/I/L/M/Q/R/S/T/V/W/Y, 143A/C/D/N/Q/S/T, 145A/C/D/E/F/G/H/I/K/L/P/Q/R/S/T/V/W, 151D/Q, 154C/D/L/R, 156C/V, 157A/C/D/E/F/G/H/I/K/L/M/N/P/Q/R/T/V/W, 159G, 160A/C/D/E/F/K/L/M/N/P/R/Q/T/V/W/Y, 161D/E/G/L/R, 162I, 163H/L, 169S, 172Q, 173F/S, 174L, 179K/S, 180H/L/M, 184A/D/G/L/M/Q/R, 185A/D/E/F/G/L/M/P/Q/R/S/T/V, 186A/R/S/T/Y, 187A, 188A/C/D/F/G/L/M/S/T/W, 190S, 191R, 192C/D/M/N, 193T, 194A/D/L/T, 198G, 199C/K/L, 212S, 214A/C/D/E/F/G/H/I/K/L/M/N/P/Q/R/T/V/W/Y, 220K/L/R, 221A/C/D/E/F/G/H/I/K/L/M/P/Q/R/T/V/W/Y, 222G, 223S, 225V, 231H/V, 232S, 233G/I/L, 235Q/R/V, 237A/G, 238Q, 239L/M, 240A/L, 242E/S, 243E/L/M/R/S/T, 245L/V, 246I/V, 249G/M/S, 250A/C/F/L/N/T, 251D/S, 252P, 253C/I/V, 254C/E, 256L/M, 258W, 262A/S, 263E/H/P/Q/R/S, 264A/C/F/I/L/N/P/R/T/V, 265C/G/R, 266H/T/Y, 267A/G/H/I/L/M/R/S/T/V/W, 268A/F/G/H/I/N/P/Q/T/V/Y, 269Q/T, 271A, 273A/C/F/L/M/S/T, 274A/G/K/L/T/V/W, 275A/V, 277D/G, 278L/N/S/V/Y, 279A/E/F/G/H/K/L/M/R/S/T/Y/V/W, 280D/K/S/T, 281C/V, 283M, 285S, 290E/G/S, 292V, 293A, 294V/W, 296M/R, 297F, 300R/V, 302G/P, 303A/V, 311A/E/D/G/K/M/Q/S, 312A/C/D/E/G/H/I/K/L/M/N/P/Q/R/T/V/W/Y, 313A/Q/S/T, 314G, 315A/C/D/E/F/G/H/I/K/L/M/Q/R/S/V/W/Y, 316K, 318N/P/R, 324A/D/E/I/R/V/W/Y, 328L/M, 336F, 339S/W, 341G, 342A/C/D/E/F/G/I/K/M/N/P/R/Q/T/V/W/Y, 343S, 345A/C/D/E/F/G/I/K/L/M/Q/R/S/V/W/Y, 346A/C/D/F/G/K/L/M/N/P/Q/R/T/V/W/Y, 355A, 358S, 360S, 364A/V, 367V, 368G/T, 369I/V/W, 370C/E/F/G/I/K/L/P/Q/R/S/V, 371L, 372A/C/F/L/R/V/Y, 373A/C/E/F/M/S/Y, 374E/G/L/R/S/W/Y, 375A/E/I/L/M/S/T/V, 377H, 381N, 382G/R/S/T, 384C, 386P/W, 389C/P, 391L/S, 392Y, 401L, 402G/*, 405L/Q, 406C/M/R/W, 409E/R/*, 410C/I/W/*, 411L/R/T/V, 412P/T/*, or 413A/C/D/S/*, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution N11K, D31G, G42W, D45Y, K50R, T53A, V84M, I99V, A100V, A126T, E128G/I/K/L/P/R/S/T/V, G129E/F/H/I/K/L/R/S/T/V, R130A/F/G/N/V, A131E/P/R/T/V/Y, T132A/C/D/E/G/P/R/V/Y, Q134A/C/D/E/G/I/L/M/N/P/S/T/V/W/Y, A135C/E/G/H/I/K/L/M/N/P/R/S/T/V/W/Y, V136C/G/I/M, H137A/D/N/S, P138Q, N139C/D/E/F/H/I/K/L/M/R/S, Q140L, N141A/C/D/E/F/G/H/I/L/M/Q/R/S/T/V/W/Y, H143A/C/D/N/Q/S/T, N145A/C/D/E/F/G/H/I/K/L/P/Q/R/S/T/V/W, E151D/Q, A154C/D/L/R, T156C/V, S157A/C/D/E/F/G/H/I/K/L/M/N/P/Q/R/T/V/W, S159G, S160A/C/D/E/F/K/L/M/N/P/R/Q/T/V/W/Y, S161D/E/G/L/R, V162I, K163H/L, T169S, D172Q, H173F/S, N174L, A179K/S, N180H/L/M, T184A/D/G/L/M/Q/R, N185A/D/E/F/G/L/M/P/Q/R/S/T/V, L186A/R/S/T/Y, G187A, R188A/C/D/F/G/L/M/S/T/W, F190S, V191R, G192C/D/M/N, G193T, N194A/D/L/T, V198G, Q199C/K/L, Y212S, S214A/C/D/E/F/G/H/I/K/L/M/N/P/Q/R/T/V/W/Y, Q220K/L/R, S221A/C/D/E/F/G/H/I/K/L/M/P/Q/R/T/V/W/Y, A222G, T223S, I225V, D231H/V, N232S, S233G/I/L, S235Q/R/V, S237A/G, L238Q, Y239L/M, G240A/L, T242E/S, Q243E/L/M/R/S/T, 1245L/V, L246I/V, A249G/M/S, D250A/C/F/L/N/T, T251D/S, D252P, A253C/I/V, D254C/E, 1256L/M, M258W, G262A/S, G263E/H/P/Q/R/S, G264A/C/F/I/L/N/P/R/T/V, Y265C/G/R, N266H/T/Y, Q267A/G/H/I/L/M/R/S/T/V/W, S268A/F/G/H/I/N/P/Q/T/V/Y, M269Q/T, E271A, V273A/C/F/L/M/S/T, Q274A/G/K/L/T/V/W, T275A/V, V277D/G, A278L/N/S/V/Y, Q279A/E/F/G/H/K/L/M/R/S/T/Y/V/W, G280D/K/S/T, T281C/V, V283M, A285S, D290E/G/S, A292V, S293A, S294V/W, S296M/R, Y297F, A300R/V, S302G/P, S303A/V, T311A/E/D/G/K/M/Q/S, S312A/C/D/E/G/H/I/K/L/M/N/P/Q/R/T/V/W/Y, N313A/Q/S/T, R314G, T315A/C/D/E/F/G/H/I/K/L/M/Q/R/S/V/W/Y, R316K, S318N/P/R, S324A/D/E/I/R/V/W/Y, V328L/M, Y336F, Y339S/W, N341G, S342A/C/D/E/F/G/I/K/M/N/P/R/Q/T/V/W/Y, R343S, T345A/C/D/E/F/G/I/K/L/M/Q/R/S/V/W/Y, S346A/C/D/F/G/K/L/M/N/P/Q/R/T/V/W/Y, P355A, A358S, V360S, 1364A/V, A367V, N368G/T, P369I/V/W, 370C/E/F/G/I/K/L/P/Q/R/S/V, I371L, S372A/C/F/L/R/V/Y, V373A/C/E/F/M/S/Y, A374E/G/L/R/S/W/Y, Q375A/E/I/L/M/S/T/V, R377H, R381N, D382G/R/S/T, A384C, E386P/W, S389C/P, T391L/S, Q392Y, H401L, A402G/*, V405L/Q, A406C/M/R/W, G409E/R/*, G410C/I/W/*, S411L/R/T/V, G412P/T/*, or G413A/C/D/S/*, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution at amino acid position 135, 137, 139, 141, 143, 157, 160, 214, 268, 273, 279, 311, 312, 315, 328, 342, 345, 346, or 372, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or amino acid residue 135C/E/G/H/I/K/L/M/N/P/R/S/T/V/W/Y, 137A/D/N/S, 139C/D/E/F/H/I/K/L/M/R/S, 141A/C/D/E/F/G/H/I/L/M/Q/R/S/T/V/W/Y, 143A/C/D/N/Q/S/T, 157A/C/D/E/F/G/H/I/K/L/M/N/P/Q/R/T/V/W, 160A/C/D/E/F/K/L/M/N/P/R/Q/T/V/W/Y, 214A/C/D/E/F/G/H/I/K/L/M/N/P/Q/R/T/V/W/Y, 268A/F/G/H/I/N/P/Q/T/V/Y, 273A/C/F/L/M/S/T, 279A/E/F/G/H/K/L/M/R/S/T/Y/V/W, 311A/E/D/G/K/M/Q/S, 312A/C/D/E/G/H/I/K/L/M/N/P/Q/R/T/V/W/Y, 315A/C/D/E/F/G/H/I/K/L/M/Q/R/S/V/W/Y, 328L/M, 342A/C/D/E/F/G/I/K/M/N/P/R/Q/T/V/W/Y, 345A/C/D/E/F/G/I/K/L/M/Q/R/S/V/W/Y, 346A/C/D/F/G/K/L/M/N/P/Q/R/T/V/W/Y, or 372A/C/F/L/R/V/Y, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution A135C/E/G/H/I/K/L/M/N/P/R/S/T/V/W/Y, H137A/D/N/S, N139C/D/E/F/H/I/K/L/M/R/S, N141A/C/D/E/F/G/H/I/L/M/Q/R/S/T/V/W/Y, H143A/C/D/N/Q/S/T, S157A/C/D/E/F/G/H/I/K/L/M/N/P/Q/R/T/V/W, S160A/C/D/E/F/K/L/M/N/P/R/Q/T/V/W/Y, S214A/C/D/E/F/G/H/I/K/L/M/N/P/Q/R/T/V/W/Y, S268A/F/G/H/I/N/P/Q/T/V/Y, V273A/C/F/L/M/S/T, Q279A/E/F/G/H/K/L/M/R/S/T/Y/V/W, T311A/E/D/G/K/M/Q/S, S312A/C/D/E/G/H/I/K/L/M/N/P/Q/R/T/V/W/Y, T315A/C/D/E/F/G/H/I/K/L/M/Q/R/S/V/W/Y, V328L/M, S342A/C/D/E/F/G/I/K/M/N/P/R/Q/T/V/W/Y, T345A/C/D/E/F/G/I/K/L/M/Q/R/S/V/W/Y, S346A/C/D/F/G/K/L/M/N/P/Q/R/T/V/W/Y, or S372A/C/F/L/R/V/Y, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution set at amino acid positions 135/141/160/311/315/372, 143/328/342/345, 139/157/268/273/312/346, or 137/139/214/279, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution set or amino acid residues 135G/141V/160L/311D/315V/372V, 143A/328L/342G/345R, 139C/157G/268G/273T/312Q/346T, or 137N/139L/214P/279M, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution set A135G/N141V/S160L/T311D/T315V/S372V, H143A/V328L/S342G/T345R, N139C/S157G/S268G/V273T/S312Q/S346T, or H137N/C139L/S214P/Q279M, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or substitution set at amino acid position(s) 185, 134, 129, 135, 184, 132, 186, 193, 263, 370, 45/134, 199, 368, 161, 141, 267, 179, 264, 160, 138, 131, 372, 151, 274, 128, 339, 313, 374, 314, 191, 324, 315, 375, 136, 220, 194, 231, 277, 369, 251, 180, 163, 343, 264/279, 279, 232, 141/300, 367, 266, 188, 130, 318, 265, 341, 190, 145, 126/192, 11/220, 192, 370/392, 99/278, 265/311, 84/159/265/279/311/370, 311/316, 342/370, 265/311/370, 192/311/316, 141/154/192, 265/311/316/342, 279/311/316, 141/265/279/311/342, 141/192/311/316/370, 141/265/311, 198/279, 392, 342/370/392, 141/198/265, 265/392, 184/267, 342, 312, 100/251, 141/220, 311/316/370, 99, 278, 405, 311/342/370, 141/198, 311/342, 141/311, 279/311/377/392, 186/198/311/342/370/392, 141/392, 311/370/392, 141/311/392, 311/370, 311/316/392, 265/311/392, 141/192, 311, 141/265/311/392, 192/311/370/392, 198/265/311/316/370, 141/186/265/311, or 141/198/265/311/370, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or substitution set, or amino acid residue(s) 185F, 134I, 129T, 135C, 184A, 129R, 132Y, 186R, 193T, 263P, 370C, 45Y/Q134W, 185M, 199K, 368T, 161E, 141T, 267L, 179S, 185V, 264L, 199C, 160M, 138Q, 131Y, 184D, 372R, 134L, 370R, 370I, 134E, 368G, 151D, 274K, 134D, 134V, 128I, 339S, 313A, 131E, 185P, 374W, 314G, 191R, 128V, 132E, 324R, 315M, 132V, 375L, 375T, 129L, 132P, 184M, 136G, 186A, 135S, 220L, 134P, 132A, 141M, 135I, 194D, 185Q, 263H, 274L, 231V, 315R, 375S, 135T, 185G, 135R, 277D, 128P, 132R, 369I, 264C, 315H, 251S, 136I, 160P, 3751, 180M, 369V, 251D, 264A, 163L, 231H, 343S, 264R/279R, 274A, 279Y, 131P, 232S, 220R, 315Q, 186T, 324V, 313S, 132D, 141R/300V, 324I, 367V, 135V, 370L, 132G, 267G, 131T, 266T, 179K, 372A, 372F, 185T, 324D, 135K, 188A, 141D, 374L, 185D, 130N, 370V, 161R, 3151, 315L, 318N, 188C, 180L, 372Y, 135P, 375E, 324A, 129K, 134M, 184G, 185A, 129H, 188D, 130F, 265C, 141W, 324W, 370E, 184R, 134A, 161L, 134T, 370G, 375A, 128G, 130V, 134N, 341G, 190S, 370P, 145R, 279H, 279S, 160Q, 370K, 126T/G192C, 374E, 128K, 160C, 186S, 11K/Q220K, 134W, 129V, 128L, 151Q, 375M, 134C, 374R, 160T, 279T, 264F, 132C, 129F, 264V, 1291, 184Q, 192M, 374S, 370F, 267A, 369W, 199L, 145M, 194A, 185S, 265R, 129S, 185R, 188W, 161G, 370G/392Y, 99V/278N, 265G/311D, 84M/159G/265G/279K/311D/370G, 311D/316K, 342N/370G, 265G/311D/370G, 192D/311D/316K, 141Q/154D/192D, 265G/311D/316K/342N, 279K/311D/316K, 141Q/265G/279K/311D/342N, 141Q/192D/311D/316K/370G, 141Q, 141Q/265G/311D, 198G/279K, 392Y, 342N/370G/392Y, 141Q/198G/265G, 265G/392Y, or 265G, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or substitution set N185F, Q134I, G129T, A135C, T184A, G129R, T132Y, L186R, G193T, G263P, D370C, D45Y/Q134W, N185M, Q199K, N368T, S161E, N141T, Q267L, A179S, N185V, G264L, Q199C, S160M, P138Q, A131Y, T184D, S372R, Q134L, D370R, D370I, Q134E, N368G, E151D, Q274K, Q134D, Q134V, E128I, Y339S, N313A, A131E, N185P, A374W, R314G, V191R, E128V, T132E, S324R, T315M, T132V, Q375L, Q375T, G129L, T132P, T184M, V136G, L186A, A135S, Q220L, Q134P, T132A, N141M, A135I, N194D, N185Q, G263H, Q274L, D231V, T315R, Q375S, A135T, N185G, A135R, V277D, E128P, T132R, P369I, G264C, T315H, 1251S, V136I, S160P, Q3751, N180M, P369V, 1251D, G264A, K163L, D231H, R343S, G264R/Q279R, Q274A, Q279Y, A131P, N232S, Q220R, T315Q, L186T, S324V, N313S, T132D, N141R/A300V, S324I, A367V, A135V, D370L, T132G, Q267G, A131T, N266T, A179K, S372A, S372F, N185T, S324D, A135K, R188A, N141D, A374L, N185D, T130N, D370V, S161R, T3151, T315L, S318N, R188C, N180L, S372Y, A135P, Q375E, S324A, G129K, Q134M, T184G, N185A, G129H, R188D, T130F, Y265C, N141W, S324W, D370E, T184R, Q134A, S161L, Q134T, D370G, Q375A, E128G, T130V, Q134N, N341G, F190S, D370P, N145R, Q279H, Q279S, S160Q, D370K, A126T/G192C, A374E, E128K, S160C, L186S, N11K/Q220K, Q134W, G129V, E128L, E151Q, Q375M, Q134C, A374R, S160T, Q279T, G264F, T132C, G129F, G264V, G1291, T184Q, G192M, A374S, D370F, Q267A, P369W, Q199L, N145M, N194A, N185S, Y265R, G129S, N185R, R188W, S161G, D370G/Q392Y, 199V/A278N, Y265G/T311D, V84M/S159G/Y265G/Q279K/T311D/D370G, T311D/R316K, S342N/D370G, Y265G/T311D/D370G, G192D/T311D/R316K, N141Q/A154D/G192D, Y265G/T311D/R316K/S342N, Q279K/T311D/R316K, N141Q/Y265G/Q279K/T311D/S342N, N141Q/G192D/T311D/R316K/D370G, N141Q, N141Q/Y265G/T311D, V198G/Q279K, Q392Y, S342N/D370G/Q392Y, N141Q/V198G/Y265G, Y265G/Q392Y, or Y265G, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or substitution set, or amino acid residue(s) 135L, 194L, 128T, 134S, 313T, 184L/267L, 185E, 342G, 374Y, 141R, 186Y, 312R, 313Q, 315V, 374G, 128S, 136A, 128R, 370Q, 267V, 188M, 188F, 263S, 188S, 339W, 100V/251S, 131V, 188T, 141L, 134Y, 267M, 264N, 134G, 185L, 370S, 267W, 279M, 267R, 264T, 279L, 263R, 136C, 145E, 188G, 130A, 192N, 188L, 312I, 129E, 315E, 145A, 267H, 372V, 130G, 267T, 274W, 136M, 372C, 194T, 375V, 135G, 267I, 141L/220R, 324E, 160L, 141S, 372L, 135Y, 141V, 141A, 131R, 135E, 324Y, 311D/316K/370G, 99V, 278N, 405Q, 311D/342N/370G, 141Q/198G, 311D/342N, 141Q/311D, 279K/311D/377H/392Y, 186Y/198G/311D/342N/370G/392Y, 141Q/392Y, 311D/370G/392Y, 141Q/311D/392Y, 311D/370G, 311D/316K/392Y, 265G/311D/392Y, 141Q/192D, 311D, 141Q/265G/311D/392Y, 192D/311D/370G/392Y, 198G/265G/311D/316K/370G, 141Q/186Y/265G/311D, or 141Q/198G/265G/311D/370G, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or substitution set A135L, N194L, E128T, Q134S, N313T, T184L/Q267L, N185E, S342G, A374Y, N141R, L186Y, S312R, N313Q, T315V, A374G, E128S, V136A, E128R, D370Q, Q267V, R188M, R188F, G263S, R188S, Y339W, A100V/I251S, A131V, R188T, N141L, Q134Y, Q267M, G264N, Q134G, N185L, D370S, Q267W, Q279M, Q267R, G264T, Q279L, G263R, V136C, N145E, R188G, T130A, G192N, R188L, S312I, G129E, T315E, N145A, Q267H, S372V, T130G, Q267T, Q274W, V136M, S372C, N194T, Q375V, A135G, Q267I, N141L/Q220R, S324E, S160L, N141S, S372L, A135Y, N141V, N141A, A131R, A135E, S324Y, T311D/R316K/D370G, I99V, A278N, V405Q, T311D/S342N/D370G, N141Q/V198G, T311D/S342N, N141Q/T311D, Q279K/T311D/R377H/Q392Y, L186Y/V198G/T311D/S342N/D370G/Q392Y, N141Q/Q392Y, T311D/D370G/Q392Y, N141Q/T311D/Q392Y, T311D/D370G, T311D/R316K/Q392Y, Y265G/T311D/Q392Y, N141Q/G192D, T311D, N141Q/Y265G/T311D/Q392Y, G192D/T311D/D370G/Q392Y, V198G/Y265G/T311D/R316K/D370G, N141Q/L186Y/Y265G/T311D, or N141Q/V198G/Y265G/T311D/D370G, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution at amino acid position 135, 137, 139, 141, 143, 145, 145, 157, 160, 214, 221, 268, 273, 279, 311, 312, 315, 315, 342, 345, 346, 402, 409, 410, 412, or 413, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or amino acid residue 135G, 135S, 135V, 135L, 135R, 135E, 135P, 135H, 135C, 135T, 135Y, 135W, 135M, 135N, 137S, 137A, 137N, 137D, 139R, 139E, 139F, 139L, 139K, 139D, 139H, 139I, 139S, 141T, 141S, 141V, 141L, 141R, 141M, 141G, 141Y, 141I, 141C, 141F, 141A, 141D, 141E, 141H, 143T, 143C, 143Q, 143A, 143D, 143S, 143N, 145Q, 145T, 145V, 145H, 145L, 145E, 145R, 145D, 145R, 145A, 145F, 145S, 145G, 145I, 145K, 145M, 145C, 145W, 157A, 157E, 157P, 157V, 157T, 157N, 157R, 157G, 157L, 157W, 157K, 157C, 157D, 157Q, 157M, 157H, 157I, 157F, 160R, 160V, 160C, 160Q, 160A, 160P, 160L, 160F, 160T, 160D, 160Y, 160W, 160E, 160K, 160N, 160M, 214G, 214M, 214L, 214Q, 214T, 214P, 214R, 214D, 214F, 214K, 214A, 214V, 214I, 214E, 214H, 214Y, 214C, 214W, 221L, 221T, 221I, 221R, 221D, 221A, 221C, 221V, 221F, 221G, 221P, 221K, 221Y, 221E, 221Q, 221M, 221H, 221W, 268V, 268Y, 268A, 268Q, 268P, 268G, 268T, 268H, 268I, 268F, 268N, 273S, 273C, 273A, 273L, 273F, 273T, 273M, 279R, 279E, 279F, 279G, 279T, 279M, 279L, 279S, 279A, 279K, 279V, 279W, 279Y, 279H, 311S, 311D, 311Q, 311M, 311K, 311G, 311A, 311E, 312V, 312D, 312G, 312R, 312W, 312M, 312L, 312N, 312E, 312A, 312T, 312Y, 312P, 312H, 312Q, 312K, 312C, 312I, 315E, 315S, 315L, 315R, 315G, 315A, 315M, 315Y, 315K, 315Q, 315D, 315W, 315C, 315V, 3151, 315H, 315F, 342F, 342G, 342R, 342Q, 342E, 342V, 342T, 342C, 342N, 3421, 342P, 342M, 342A, 342W, 342K, 342D, 342Y, 345G, 345R, 345L, 345V, 345A, 345M, 345W, 345I, 345S, 345E, 345Y, 345D, 345Q, 345F, 345C, 345K, 346T, 346Q, 346V, 346R, 346P, 346L, 346D, 346W, 346G, 346A, 346C, 346M, 346F, 346N, 346Y, 346K, 402*, 409*, 410*, 412*, or 413*, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution A135G, A135S, A135V, A135L, A135R, A135E, A135P, A135H, A135C, A135T, A135Y, A135W, A135M, A135N, H137S, H137A, H137N, H137D, N139R, N139E, N139F, N139L, N139K, N139D, N139H, N139I, N139S, N141T, N141S, N141V, N141L, N141R, N141M, N141G, N141Y, N141I, N141C, N141F, N141A, N141D, N141E, N141H, H143T, H143C, H143Q, H143A, H143D, H143S, H143N, N145Q, N145T, N145V, N145H, N145L, N145E, N145R, N145D, N145R, N145A, N145F, N145S, N145G, N145I, N145K, N145M, N145C, N145W, S157A, S157E, S157P, S157V, S157T, S157N, S157R, S157G, S157L, S157W, S157K, S157C, S157D, S157Q, S157M, S157H, S157I, S157F, S160R, S160V, S160C, S160Q, S160A, S160P, S160L, S160F, S160T, S160D, S160Y, S160W, S160E, S160K, S160N, S160M, S214G, S214M, S214L, S214Q, S214T, S214P, S214R, S214D, S214F, S214K, S214A, S214V, S214I, S214E, S214H, S214Y, S214C, S214W, S221L, S221T, S221I, S221R, S221D, S221A, S221C, S221V, S221F, S221G, S221P, S221K, S221Y, S221E, S221Q, S221M, S221H, S221W, S268V, S268Y, S268A, S268Q, S268P, S268G, S268T, S268H, S268I, S268F, S268N, V273S, V273C, V273A, V273L, V273F, V273T, V273M, Q279R, Q279E, Q279F, Q279G, Q279T, Q279M, Q279L, Q279S, Q279A, Q279K, Q279V, Q279W, Q279Y, Q279H, T311S, T311D, T311Q, T311M, T311K, T311G, T311A, T311E, S312V, S312D, S312G, S312R, S312W, S312M, S312L, S312N, S312E, S312A, S312T, S312Y, S312P, S312H, S312Q, S312K, S312C, S312I, T315E, T315S, T315L, T315R, T315G, T315A, T315M, T315Y, T315K, T315Q, T315D, T315W, T315C, T315V, T3151, T315H, T315F, S342F, S342G, S342R, S342Q, S342E, S342V, S342T, S342C, S342N, S3421, S342P, S342M, S342A, S342W, S342K, S342D, S342Y, T345G, T345R, T345L, T345V, T345A, T345M, T345W, T345I, T345S, T345E, T345Y, T345D, T345Q, T345F, T345C, T345K, S346T, S346Q, S346V, S346R, S346P, S346L, S346D, S346W, S346G, S346A, S346C, S346M, S346F, S346N, S346Y, S346K, A402*, G409*, G410*, G412*, or G413*, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or substitution set at amino acid position(s) 242, 157, 250, 373, 243, 336, 187, 240, 280, 271, 237, 386, 382, 328, 42, 391, 381, 275, 249, 239, 384, 139, 364, 346, 389, 254, 246, 345, 360, 303, 300, 269, 135/141/372, 311/315/372, 136/141/311, 141/188, 135/136, 135/141/315, 372, 135/141/160/267/372, 135/136/141/160/185/188/267/311/315, 160/185, 135/141/188/279/311, 135/136/141, 135/136/141/372, 135/141/160/185/267/279, 135/141/160/267, 141/188/311/372, 160/185/188/279/311, 136/141/279, 135/136/141/160/185/188, 141/372, 135/136/141/311, 185/311/315/372, 135/141/188, 136/185, 135/141, 135/136/141/279/315/372, 135/311/315, 141, 311/372, 188/311, 135/141/188/372, 141/160/279, 313/392, 342/392, 279/392, 128, 198/342, 313, 128/312, 50, 145/263, 313/342, 279/312, 312/392, 279/342, 128/342, 342, 263, 143, 262, 156, 169, 143/237, 136/160/185/267/311/372, 135/160/311/372, 135/141/311/315, 141/311/315, 136/141/160/185/188/311/315/372, 135/141/311/315/372, 135/141/160/185/311/315, 135/141/267/311/315/372, 135/136/141/160/311/315, 135/136/141/279, 135/141/267/279/311/315, 135/141/160, 135/141/160/311/315/372, 135/141/160/311/315, 135/136/141/188/311, 141/160/311, 135/141/160/279/311/315/372, 141/160/185/279/311/372, 135/136/141/160/315/372, 135/136/160/279/311/372, 128/279/312/342, 128/198/312/342, 263/342, 145/263/279/312/342/392, or 128/145/198/312/313/392, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or substitution set, or amino acid residue(s) 242E, 157V, 250N, 373F, 243R, 336F, 187A, 240A, 280K, 271A, 237G, 386W, 382G, 280S, 373Y, 328M, 157R, 157A, 42W, 243E, 382S, 391L, 381N, 243M, 275V, 157I, 373S, 157T, 280D, 249G, 239L, 384C, 139M, 240L, 243T, 250L, 250A, 382T, 364A, 346V, 373M, 389P, 373C, 382R, 373E, 254E, 246I, 250F, 280T, 373A, 139K, 345I, 360S, 275A, 249M, 364V, 303V, 300R, 239M, 269T, 135G/141Q/372L, 311D/315V/372L, 136M/141V/311D, 141V/188M, 135E/136M, 136M/141Q/311D, 135E/141V/315V, 372V, 135E/141V/160L/267I/372V, 135G/136M/141V/160L/185E/188M/267I/311D/315V, 135G/136M, 160L/185E, 135E/141V/188M/279M/311D, 135G/136M/141Q, 135G/136M/141Q/372L, 135G/141V/160L/185E/267I/279M, 135G/141V/160L/267I, 141Q/188M/311D/372V, 160L/185E/188M/279M/311D, 136M/141V/279M, 135G/136M/141V/160L/185E/188L, 141Q/372V, 135E/136M/141Q/311D, 185E/311D/315V/372V, 135G/141V/188M, 136M/185E, 135E/141Q, 135E/136M/141Q/279M/315V/372L, 135G/311D/315V, 141V, 135G/141V, 311D/372L, 188M/311D, 135E/141V/188L/372L, 141V/160L/279M, 313Q/392Y, 342G/392Y, 279L/392Y, 128T, 198G/342G, 313Q, 128T/312I, 50R, 145E/263S, 313Q/342G, 279L/312I, 312I/392Y, 279K/342G, 279K/392Y, 128T/342G, 342G, or 263S, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or substitution set T242E, S157V, D250N, V373F, Q243R, Y336F, G187A, G240A, G280K, E271A, S237G, E386W, D382G, G280S, V373Y, V328M, S157R, S157A, G42W, Q243E, D382S, T391L, R381N, Q243M, T275V, S157I, V373S, S157T, G280D, A249G, Y239L, A384C, N139M, G240L, Q243T, D250L, D250A, D382T, I364A, S346V, V373M, S389P, V373C, D382R, V373E, D254E, L246I, D250F, G280T, V373A, N139K, T345I, V360S, T275A, A249M, I364V, S303V, A300R, Y239M, M269T, A135G/N141Q/S372L, T311D/T315V/S372L, V136M/N141V/T311D, N141V/R188M, A135E/V136M, V136M/N141Q/T311D, A135E/N141V/T315V, S372V, A135E/N141V/S160L/Q267I/S372V, A135G/V136M/N141V/S160L/N185E/R188M/Q267I/T311D/T315V, A135G/V136M, S160L/N185E, A135E/N141V/R188M/Q279M/T311D, A135G/V136M/N141Q, A135G/V136M/N141Q/S372L, A135G/N141V/S160L/N185E/Q267I/Q279M, A135G/N141V/S160L/Q267I, N141Q/R188M/T311D/S372V, S160L/N185E/R188M/Q279M/T311D, V136M/N141V/Q279M, A135G/V136M/N141V/S160L/N185E/R188L, N141Q/S372V, A135E/V136M/N141Q/T311D, N185E/T311D/T315V/S372V, A135G/N141V/R188M, V136M/N185E, A135E/N141Q, A135E/V136M/N141Q/Q279M/T315V/S372L, A135G/T311D/T315V, N141V, A135G/N141V, T311D/S372L, R188M/T311D, A135E/N141V/R188L/S372L, N141V/S160L/Q279M, N313Q/Q392Y, S342G/Q392Y, Q279L/Q392Y, E128T, V198G/S342G, N313Q, E128T/S312I, K50R, N145E/G263S, N313Q/S342G, Q279L/S312I, S312I/Q392Y, Q279K/S342G, Q279K/Q392Y, E128T/S342G, S342G, or G263S, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or substitution set, or amino acid residue(s) 139C, 345R, 243L, 143A, 249S, 262S, 139R, 269Q, 328L, 157G, 156V, 242S, 139L, 262A, 169S, 346T, 143N/237A, 136M/160L/185E/267I/311D/372L, 135E/160L/311D/372L, 135G/141V/311D/315V, 141V/311D/315V, 136M/141V/160L/185E/188M/311D/315V/372V, 135G/141V/311D/315V/372L, 135G/141Q/160L/185E/311D/315V, 135G/141V/267I/311D/315V/372V, 135G/136M/141V/160L/311D/315V, 135G/136M/141V/279M, 135G/141Q/267I/279M/311D/315V, 135E/141V/311D/315V/372V, 135E/141Q/160L, 135G/141Q/311D/315V, 135G/141V/160L/311D/315V/372V, 135G/141V/160L/311D/315V, 135G/141Q/267I/311D/315V/372L, 135G/136M/141V/188M/311D, 141V/160L/311D, 135E/141V/160L/279M/311D/315V/372L, 141V/160L/185E/279M/311D/372V, 135E/141V/311D/315V, 135G/136M/141V/160L/315V/372V, 135E/141V/160L, 135E/136M/160L/279M/311D/372V, 128T/279K/312I/342G, 128T/198G/312I/342G, 263S/342G, 145E/263S/279L/312I/342G/392Y, or 128T/145E/198G/312I/313Q/392Y, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or substitution set N139C, T345R, Q243L, H143A, A249S, G262S, N139R, M269Q, V328L, S157G, T156V, T242S, N139L, G262A, T169S, S346T, H143N/S237A, V136M/S160L/N185E/Q267I/T311D/S372L, A135E/S160L/T311D/S372L, A135G/N141V/T311D/T315V, N141V/T311D/T315V, V136M/N141V/S160L/N185E/R188M/T311D/T315V/S372V, A135G/N141V/T311D/T315V/S372L, A135G/N141Q/S160L/N185E/T311D/T315V, A135G/N141V/Q267I/T311D/T315V/S372V, A135G/V136M/N141V/S160L/T311D/T315V, A135G/V136M/N141V/Q279M, A135G/N141Q/Q267I/Q279M/T311D/T315V, A135E/N141V/T311D/T315V/S372V, A135E/N141Q/S160L, A135G/N141Q/T311D/T315V, A135G/N141V/S160L/T311D/T315V/S372V, A135G/N141V/S160L/T311D/T315V, A135G/N141Q/Q267I/T311D/T315V/S372L, A135G/V136M/N141V/R188M/T311D, N141V/S160L/T311D, A135E/N141V/S160L/Q279M/T311D/T315V/S372L, N141V/S160L/N185E/Q279M/T311D/S372V, A135E/N141V/T311D/T315V, A135G/V136M/N141V/S160L/T315V/S372V, A135E/N141V/S160L, A135E/V136M/S160L/Q279M/T311D/S372V, E128T/Q279K/S312I/S342G, E128T/V198G/S312I/S342G, G263S/S342G, N145E/G263S/Q279L/S312I/S342G/Q392Y, or E128T/N145E/V198G/S312I/N313Q/Q392Y, wherein the amino acid positions are relative to the reference sequence corresponding to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution set at amino acid position(s) 135/141/160/311/315/372/411, 135/141/160/311/315/372/402, 135/141/160/285/311/315/372, 135/141/160/245/311/315/372, 135/141/160/266/311/315/372, 135/141/160/311/315/355/372, 135/141/160/258/311/315/372, 135/141/160/222/311/315/372, 135/140/141/160/311/315/372, 135/141/160/268/311/315/372, 135/141/160/225/311/315/372, 135/141/160/283/311/315/372, 135/141/160/311/315/372/406, 135/141/160/311/315/372/410, 135/141/143/145/160/243/311/312/315/372, 135/139/141/143/145/157/160/311/312/315/372, 135/139/141/157/160/311/315/345/372, 135/139/141/160/269/311/315/372, 135/141/156/157/160/311/315/342/346/372, 135/139/141/143/160/311/315/372, 135/141/160/269/311/315/372, 135/139/141/160/243/311/315/328/372, 135/141/160/269/311/315/328/372, 135/141/143/145/160/169/311/315/372, 135/141/160/311/315/328/372, 135/141/143/145/160/262/311/315/372, 135/141/145/160/262/311/312/315/328/372, 135/139/141/156/157/160/311/315/372, 135/139/141/145/160/311/312/315/372, 135/141/160/311/312/315/372, 135/139/141/160/311/315/372, 135/139/141/160/311/312/315/372, 135/139/141/156/160/311/315/372, 135/139/141/143/145/160/243/311/315/372, 135/141/145/157/160/311/315/372, 135/141/145/160/311/315/346/372, 135/141/145/160/262/311/312/315/328/345/346/372, 135/141/145/160/262/311/315/372, 135/141/160/311/312/315/342/372, 135/141/143/160/243/311/315/372, 135/139/141/160/311/315/345/372, 135/141/160/311/315/342/372, 135/141/143/145/160/262/311/315/342/372, 135/139/141/143/160/169/311/315/372, 135/139/141/143/145/160/311/312/315/372, 135/141/160/169/311/315/372, 135/139/141/145/160/262/311/312/315/328/342/345/346/372, 135/139/141/160/311/315/328/372, 135/139/141/160/243/311/315/372, 135/139/141/143/160/311/315/328/372, 135/139/141/143/160/243/311/315/372, 135/139/141/145/160/311/315/372, 135/141/145/160/311/312/315/372, 135/141/145/160/169/311/315/372, 135/139/141/143/157/160/311/312/315/372, 84/135/139/143/141/160/311/315/372, 135/141/145/160/269/311/315/372, 135/141/143/145/157/160/269/311/312/315/328/372, 135/141/143/145/160/269/311/315/372, 135/141/157/160/311/315/372, 135/139/141/143/160/311/312/315/372, 135/141/160/256/311/315/372, 135/141/160/273/311/315/372, 135/141/160/311/315/372/409, 135/141/160/172/311/315/372, 135/141/160/311/315/372/401, 135/141/160/281/311/315/372, 135/141/160/253/311/315/372, 135/141/143/145/160/243/311/315/328/372, 135/141/145/160/311/315/372, 135/139/141/143/145/160/311/315/328/342/345/372, 135/141/143/160/311/315/328/342/345/372, 135/141/145/160/311/315/342/345/372, 135/141/143/160/311/315/372, 135/141/139/143/160/311/315/372, 135/139/141/145/160/311/315/328/342/345/372, 135/141/143/145/160/169/311/312/315/328/345/346/372, 135/141/143/160/243/311/315/328/342/345/346/372, 135/139/141/143/157/160/169/311/315/328/346/372, 135/143/141/145/156/160/311/312/315/328/372, 135/139/141/145/157/160/311/312/315/328/372, 135/141/143/160/311/315/328/342/345/346/372, 135/141/143/145/160/311/315/372, 135/141/143/145/160/311/312/315/342/345/372, or 135/141/143/145/160/311/315/328/372, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution set or amino acid residues 135G/141V/160L/311D/315V/372V/S411V, 135G/141V/160L/311D/315V/372V/411R, 135G/141V/160L/311D/315V/372V/402G, 135G/141V/160L/285S/311D/315V/372V, 135G/141V/160L/245V/311D/315V/372V, 135G/141V/160L/266H/311D/315V/372V, 135G/141V/160L/311D/315V/355A/372V, 135G/141V/160L/258W/311D/315V/372V, 135G/141V/160L/222G/311D/315V/372V, 135G/140L/141V/160L/311D/315V/372V, 135G/141V/160L/268T/311D/315V/372V, 135G/141V/160L/311D/315V/372V/411L, 135G/141V/160L/225V/311D/315V/372V, 135G/141V/160L/245L/311D/315V/372V, 135G/141V/160L/283M/311D/315V/372V, 135G/141V/160L/311D/315V/372V/406W, 135G/141V/160L/311D/315V/372V/410C, 135G/141V/160L/311D/315V/372V/406C, 135G/141V/143A/145E/160L/243L/311D/312I/315V/372V, 135G/139L/141V/143A/145E/157G/160L/311D/312I/315V/372V, 135G/139C/141V/157G/160L/311D/315V/345R/372V, 135G/139C/141V/160L/269Q/311D/315V/372V, 135G/141V/156V/157G/160L/311D/315V/342G/346T/372V, 135G/139L/141V/143A/160L/311D/315V/372V, 135G/141V/160L/269Q/311D/315V/372V, 135G/139L/141V/160L/243L/311D/315V/328L/372V, 135G/141V/160L/269Q/311D/315V/328L/372V, 135G/141V/143A/145E/160L/169S/311D/315V/372V, 135G/141V/160L/311D/315V/328L/372V, 135G/141V/143A/145E/160L/262S/311D/315V/372V, 135G/141V/145E/160L/262S/311D/312I/315V/328L/372V, 135G/139L/141V/156V/157G/160L/311D/315V/372V, 135G/139C/141V/145E/160L/311D/312I/315V/372V, 135G/141V/160L/311D/312I/315V/372V, 135G/139C/141V/160L/311D/315V/372V, 135G/139C/141V/160L/311D/312I/315V/372V, 135G/139C/141V/156V/160L/311D/315V/372V, 135G/139C/141V/143A/145E/160L/243L/311D/315V/372V, 135G/141V/145E/157G/160L/311D/315V/372V, 135G/141V/145E/160L/311D/315V/346T/372V, 135G/141V/145E/160L/262A/311D/312I/315V/328L/345R/346T/372V, 135G/141V/145E/160L/262A/311D/315V/372V, 135G/141V/160L/311D/312I/315V/342G/372V, 135G/141V/143A/160L/243L/311D/315V/372V, 135G/139C/141V/160L/311D/315V/345R/372V, 135G/141V/160L/311D/315V/342G/372V, 135G/141V/143A/145E/160L/262S/311D/315V/342G/372V, 135G/139L/141V/143A/160L/169S/311D/315V/372V, 135G/139C/141V/143A/145E/160L/311D/312I/315V/372V, 135G/141V/160L/169S/311D/315V/372V, 135G/139L/141V/145E/160L/262A/311D/312I/315V/328L/342G/345R/346T/372V, 135G/139C/141V/160L/311D/315V/328L/372V, 135G/139L/141V/160L/243L/311D/315V/372V, 135G/139L/141V/143A/160L/311D/315V/328L/372V, 135G/139L/141V/160L/311D/315V/372V, 135G/139C/141V/143A/160L/243L/311D/315V/372V, 135G/139C/141V/145E/160L/311D/315V/372V, 135G/141V/145E/160L/311D/312I/315V/372V, 135G/141V/145E/160L/169S/311D/315V/372V, 135G/139C/141V/143A/157G/160L/311D/312I/315V/372V, 84M/135G/139C/143A/141V/160L/311D/315V/372V, 135G/141V/145E/160L/269Q/311D/315V/372V, 135G/141V/143A/145E/157G/160L/269Q/311D/312I/315V/328L/372V, 135G/141V/143A/145E/160L/269Q/311D/315V/372V, 135G/141V/157G/160L/311D/315V/372V, or 135G/139L/141V/143A/160L/311D/312I/315V/372V, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution set or amino acid residues 135G/141V/160L/256L/311D/315V/372V, 135G/141V/160L/311D/315V/372V/411T, 135G/141V/160L/273F/311D/315V/372V, 135G/141V/160L/311D/315V/372V/409E, 135G/141V/160L/268G/311D/315V/372V, 135G/141V/160L/172Q/311D/315V/372V, 135G/141V/160L/311D/315V/372V/409R, 135G/141V/160L/311D/315V/372V/401L, 135G/141V/160L/281C/311D/315V/372V, 135G/141V/160L/311D/315V/372V/410W, 135G/141V/160L/253V/311D/315V/372V, 135G/141V/160L/311D/315V/372V/406M, 135G/141V/160L/273T/311D/315V/372V, 135G/141V/160L/311D/315V/372V/406R, 135G/141V/160L/256M/311D/315V/372V, 135G/141V/160L/311D/315V/372V/410I, 135G/141V/160L/273M/311D/315V/372V, 135G/141V/160L/273L/311D/315V/372V, 135G/141V/143A/145E/160L/243L/311D/315V/328L/372V, 135G/141V/145E/160L/311D/315V/372V, 135G/139C/141V/143A/145E/160L/311D/315V/328L/342G/345R/372V, 135G/139L/141V/145E/160L/311D/315V/372V, 135G/141V/143A/160L/311D/315V/328L/342G/345R/372V, 135G/141V/145E/160L/311D/315V/342G/345R/372V, 135G/141V/143A/160L/311D/315V/372V, 135G/141V/139C/143A/160L/311D/315V/372V, 135G/139C/141V/145E/160L/311D/315V/328L/342G/345R/372V, 135G/141V/143A/145E/160L/169S/311D/312I/315V/328L/345R/346T/372V, 135G/141V/143A/160L/243L/311D/315V/328L/342G/345R/346T/372V, 135G/139C/141V/143A/157G/160L/169S/311D/315V/328L/346T/372V, 135G/143A/141V/145E/156V/160L/311D/312I/315V/328L/372V, 135G/139C/141V/145E/157G/160L/311D/312I/315V/328L/372V, 135G/141V/143A/160L/311D/315V/328L/342G/345R/346T/372V, 135G/139C/141V/143A/160L/311D/315V/328L/372V, 135G/141V/143A/145E/160L/311D/315V/372V, 135G/141V/143A/145E/160L/311D/312I/315V/342G/345R/372V, or 135G/141V/143A/145E/160L/311D/315V/328L/372V, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution set at amino acid positions 135/141/143/160/279/311/315/328/342/345/372, 135/141/143/160/250/311/315/328/342/345/372, 135/141/143/154/160/311/315/328/342/345/372, 135/141/143/160/214/311/315/328/342/345/372, 135/141/143/160/249/311/315/328/342/345/372, 135/141/143/160/275/311/315/328/342/345/372, 135/137/141/143/160/311/315/328/342/345/372, 135/141/143/160/161/311/315/328/342/345/372, 135/141/143/160/180/311/315/328/342/345/372, 135/141/143/160/174/311/315/328/342/345/372, 135/139/141/143/160/311/315/328/342/345/372, 135/141/143/160/254/311/315/328/342/345/372, 135/141/143/145/160/311/315/328/342/345/372, 135/141/143/160/278/311/315/328/342/345/372, 135/136/141/143/160/311/315/328/342/345/372, 135/141/143/154/160/311/315/328/342/345/372/413, 135/141/143/160/294/311/315/328/342/345/372, 135/141/143/160/237/311/315/328/342/345/372, 135/141/143/160/311/315/328/342/345/372, 135/141/143/160/274/311/315/328/342/345/372, 135/141/143/160/264/311/315/328/342/345/372, 135/141/143/160/185/311/315/328/342/345/372, 135/141/143/160/277/311/315/328/342/345/372, 135/141/143/160/293/311/315/328/342/345/372, 135/141/143/160/233/311/315/328/342/345/372, 135/141/143/160/173/311/315/328/342/345/372, 135/141/143/160/311/312/315/328/342/345/372, 135/141/143/160/302/311/315/328/342/345/372, 135/141/143/160/238/311/315/328/342/345/372, 141/143/160/311/315/328/342/345/372, 135/141/143/160/221/311/315/328/342/345/372, 135/141/143/160/290/311/315/328/342/345/372, 135/141/143/160/263/311/315/328/342/345/372, 135/141/143/160/267/311/315/328/342/345/372, 135/141/143/160/239/311/315/328/342/345/372, 135/141/143/160/163/311/315/328/342/345/372, 135/141/143/160/292/311/315/328/342/345/372, 135/141/143/160/246/311/315/328/342/345/372, 135/141/143/160/243/311/315/328/342/345/372, 135/141/143/160/235/311/315/328/342/345/372, 135/141/143/156/160/311/315/328/342/345/372, 135/141/143/160/223/311/315/328/342/345/372, 135/141/143/160/278/311/315/328/342/345/372/413, 135/141/143/160/297/311/315/328/342/345/372, 135/141/143/160/194/311/315/328/342/345/372, 135/141/143/160/251/311/315/328/342/345/372, 135/141/143/145/157/160/253/268/273/281/311/312/315/328/342/345/346/411/372, 135/139/141/143/160/311/315/328/342/345/346/372, 135/141/143/160/253/311/315/328/342/345/372, 135/141/143/160/311/315/328/342/345/346/372/411, 135/141/143/160/253/311/315/328/342/345/346/372, 135/141/143/160/311/312/315/328/342/345/346/372, 135/141/143/160/273/311/312/315/328/342/345/372, 135/141/143/160/253/281/311/315/328/342/345/372, 135/141/143/157/160/253/273/311/312/315/328/342/345/346/372/411, 135/139/141/143/157/160/253/268/273/281/311/312/315/328/342/345/346/372, 135/141/143/160/253/273/311/315/328/342/345/372/411, 135/139/141/143/160/253/268/273/281/311/315/328/342/345/372, 135/139/141/143/157/160/311/315/328/342/345/372/411, 135/141/143/157/160/311/315/328/342/345/372, 135/141/143/160/273/311/315/328/342/345/372, 135/139/141/143/160/253/268/273/281/311/312/315/328/342/345/372/411, 135/141/143/157/160/253/311/315/328/342/345/372/411, 135/139/141/143/145/160/253/311/315/328/342/345/346/372, 135/139/141/143/157/160/253/273/311/312/315/328/342/345/372, 135/139/141/143/157/160/268/273/311/312/315/328/342/345/346/372, 135/141/143/157/160/273/311/312/315/328/342/345/346/372, 135/139/141/143/160/311/315/328/342/345/372/411, 135/139/141/143/160/253/268/273/281/311/312/315/328/342/345/346/372/411, 135/141/143/157/160/273/311/315/328/342/345/346/372/411, 135/139/141/143/145/157/160/162/253/273/281/311/312/315/328/342/345/372, 135/139/141/143/160/253/273/281/311/312/315/328/342/345/372, 135/141/143/157/160/253/268/273/281/311/312/315/328/342/345/372, 135/139/141/143/160/253/268/311/315/328/342/345/372, 135/139/141/143/157/160/311/312/315/328/342/345/372, 135/141/143/160/253/273/281/311/315/328/342/345/346/372, 135/141/143/157/160/253/311/312/315/328/342/345/346/372/411, 135/141/143/157/160/273/311/312/315/328/342/345/346/372/411, 135/139/141/143/145/157/160/253/268/281/311/312/315/328/342/345/372, 135/139/141/143/160/273/311/312/315/328/342/345/346/372, 135/141/143/157/160/253/268/273/311/312/315/328/342/345/346/372, 135/139/141/143/160/268/311/315/328/342/345/346/372, 135/141/143/160/268/273/311/312/315/328/342/345/346/372, 135/139/141/143/157/160/253/268/273/311/312/315/328/342/345/372, 135/139/141/143/157/160/253/311/315/328/342/345/372, 135/139/141/143/160/253/281/311/315/328/342/345/372, 135/139/141/143/157/160/253/268/273/311/315/328/342/345/372, 135/141/143/160/253/311/312/315/328/342/345/372/411, or 135/139/141/143/160/268/273/311/315/328/342/345/372, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution set or amino acid residues 135G/141V/143A/160L/279Y/311D/315V/328L/342G/345R/372V, 135G/141V/143A/160L/250T/311D/315V/328L/342G/345R/372V, 135G/141V/143A/154C/160L/311D/315V/328L/342G/345R/372V, 135G/141V/143A/160L/214Y/311D/315V/328L/342G/345R/372V, 135G/141V/143A/160L/249S/311D/315V/328L/342G/345R/372V, 135G/141V/143A/160L/275A/311D/315V/328L/342G/345R/372V, 135G/137A/141V/143A/160L/311D/315V/328L/342G/345R/372V, 135G/141V/143A/160L/161D/311D/315V/328L/342G/345R/372V, 135G/141V/143A/160L/180H/311D/315V/328L/342G/345R/372V, 135G/141V/143A/160L/174L/311D/315V/328L/342G/345R/372V, 135G/139K/141V/143A/160L/311D/315V/328L/342G/345R/372V, 135G/141V/143A/160L/254C/311D/315V/328L/342G/345R/372V, 135G/141V/143A/145E/160L/311D/315V/328L/342G/345R/372V, 135G/141V/143A/160L/278V/311D/315V/328L/342G/345R/372V, 135G/136M/141V/143A/160L/311D/315V/328L/342G/345R/372V, 135G/141V/143A/154L/160L/311D/315V/328L/342G/345R/372V/413D, 135G/141V/143A/160L/294V/311D/315V/328L/342G/345R/372V, 135G/141V/143A/154R/160L/311D/315V/328L/342G/345R/372V, 135G/141V/143A/160L/237A/311D/315V/328L/342G/345R/372V, 135G/141V/143A/160L/311D/315V/328L/342G/345R/372V, 135G/141V/143A/160L/274G/311D/315V/328L/342G/345R/372V, 135G/141V/143A/160L/264P/311D/315V/328L/342G/345R/372V, 135G/141V/143A/160L/274T/311D/315V/328L/342G/345R/372V, 135G/141V/143A/160L/278N/311D/315V/328L/342G/345R/372V, 135G/141V/143A/160L/214A/311D/315V/328L/342G/345R/372V, 135G/141V/143A/160L/185G/311D/315V/328L/342G/345R/372V, 135G/141V/143A/160L/214V/311D/315V/328L/342G/345R/372V, 135G/141V/143A/160L/279T/311D/315V/328L/342G/345R/372V, 135G/141V/143A/160L/277G/311D/315V/328L/342G/345R/372V, 135G/141V/143A/160L/264I/311D/315V/328L/342G/345R/372V, 135G/141V/143A/160L/293A/311D/315V/328L/342G/345R/372V, 135G/141V/143A/160L/233L/311D/315V/328L/342G/345R/372V, 135G/141V/143A/160L/278Y/311D/315V/328L/342G/345R/372V, 135G/141V/143A/160L/173F/311D/315V/328L/342G/345R/372V, 135G/141V/143A/160L/274L/311D/315V/328L/342G/345R/372V, 135G/141V/143A/160L/311D/312C/315V/328L/342G/345R/372V, 135G/141V/143A/160L/279L/311D/315V/328L/342G/345R/372V, 135G/141V/143A/160L/302G/311D/315V/328L/342G/345R/372V, 135G/141V/143A/160L/238Q/311D/315V/328L/342G/345R/372V, 135G/141V/143A/160L/294W/311D/315V/328L/342G/345R/372V, 141V/143A/160L/311D/315V/328L/342G/345R/372V, 135G/141V/143A/160L/221E/311D/315V/328L/342G/345R/372V, 135G/141V/143A/160L/290S/311D/315V/328L/342G/345R/372V, 135G/141V/143A/160L/278S/311D/315V/328L/342G/345R/372V, 135G/141V/143A/160L/263Q/311D/315V/328L/342G/345R/372V, 135G/141V/143A/160L/263H/311D/315V/328L/342G/345R/372V, 135G/141V/143A/160L/278L/311D/315V/328L/342G/345R/372V, 135G/141V/143A/160L/263E/311D/315V/328L/342G/345R/372V, 135G/141V/143A/154L/160L/311D/315V/328L/342G/345R/372V, 135G/139M/141V/143A/160L/311D/315V/328L/342G/345R/372V, 135G/137S/141V/143A/160L/311D/315V/328L/342G/345R/372V, 135G/141V/143A/160L/233G/311D/315V/328L/342G/345R/372V, 135G/139F/141V/143A/160L/311D/315V/328L/342G/345R/372V, 135G/141V/143A/160L/267I/311D/315V/328L/342G/345R/372V, 135G/141V/143A/160L/221L/311D/315V/328L/342G/345R/372V, 135G/141V/143A/160L/173S/311D/315V/328L/342G/345R/372V, 135G/141V/143A/160L/302P/311D/315V/328L/342G/345R/372V, 135G/141V/143A/160L/221V/311D/315V/328L/342G/345R/372V, 135G/141V/143A/160L/239M/311D/315V/328L/342G/345R/372V, 135G/141V/143A/160L/290G/311D/315V/328L/342G/345R/372V, 135G/141V/143A/160L/163H/311D/315V/328L/342G/345R/372V, 135G/141V/143A/160L/292V/311D/315V/328L/342G/345R/372V, 135G/141V/143A/160L/246V/311D/315V/328L/342G/345R/372V, 135G/141V/143A/160L/214N/311D/315V/328L/342G/345R/372V, 135G/141V/143A/160L/243S/311D/315V/328L/342G/345R/372V, 135G/141V/143A/160L/233I/311D/315V/328L/342G/345R/372V, 135G/141V/143A/160L/235Q/311D/315V/328L/342G/345R/372V, 135G/141V/143A/145D/160L/311D/315V/328L/342G/345R/372V, 135G/141V/143A/160L/274V/311D/315V/328L/342G/345R/372V, 135G/141V/143A/160L/279M/311D/315V/328L/342G/345R/372V, 135G/141V/143A/160L/185S/311D/315V/328L/342G/345R/372V, 135G/141V/143A/160L/279K/311D/315V/328L/342G/345R/372V, 135G/141V/143A/145W/160L/311D/315V/328L/342G/345R/372V, 135G/141V/143A/160L/290E/311D/315V/328L/342G/345R/372V, 135G/141V/143A/160L/214P/311D/315V/328L/342G/345R/372V, 135G/141V/143A/156V/160L/311D/315V/328L/342G/345R/372V, 135G/141V/143A/156C/160L/311D/315V/328L/342G/345R/372V, 135G/141V/143A/160L/223S/311D/315V/328L/342G/345R/372V, 135G/141V/143A/160L/278V/311D/315V/328L/342G/345R/372V/413D, 135G/141V/143A/160L/250C/311D/315V/328L/342G/345R/372V, 135G/141V/143A/160L/267S/311D/315V/328L/342G/345R/372V, 135G/141V/143A/160L/297F/311D/315V/328L/342G/345R/372V, 135G/141V/143A/160L/221Q/311D/315V/328L/342G/345R/372V, 135G/141V/143A/160L/194D/311D/315V/328L/342G/345R/372V, 135G/141V/143A/160L/311D/315V/328L/342G/345R/372V, 135G/141V/143A/160L/251T/311D/315V/328L/342G/345R/372V, 135G/141V/143A/145P/157R/160L/253I/268G/273T/281V/311D/312Q/315V/328L/342G/345R/346T/411T/372V, 135G/139C/141V/143A/160L/311D/315V/328L/342G/345R/346A/372V, 135G/141V/143A/160L/253V/311D/315V/328L/342G/345R/372V, 135G/141V/143A/160L/311D/315V/328L/342G/345R/346T/372V/411T, 135G/141V/143A/160L/253V/311D/315V/328L/342G/345R/346T/372V, 135G/139C/141V/143A/160L/311D/315V/328L/342G/345R/346T/372V, 135G/141V/143A/160L/311D/312Q/315V/328L/342G/345R/346T/372V, 135G/141V/143A/160L/273T/311D/S312Q/315V/328L/342G/345R/372V, 135G/141V/143A/160L/253I/281V/311D/315V/328L/342G/345R/372V, 135G/141V/143A/157R/160L/253V/273T/311D/312Q/315V/328L/342G/345R/346T/372V/41 IT, 135G/139C/141V/143A/157K/160L/253V/268G/273F/281V/311D/312Q/315V/328L/342G/345R/346A/372V, 135G/141V/143A/160L/253V/273T/311D/315V/328L/342G/345R/372V/411T, 135G/139C/141V/143A/160L/253I/268F/273T/281V/311D/315V/328L/342G/345R/372V, 135G/139C/141V/143A/157R/160L/311D/315V/328L/342G/345R/372V/411T, 135G/141V/143A/157G/160L/311D/315V/328L/342G/345R/372V, 135G/141V/143A/160L/273T/311D/315V/328L/342G/345R/372V, 135G/139C/141V/143A/160L/253V/268G/273F/281V/311D/312Q/315V/328L/342G/345R/372V/41 iT, 135G/141V/143A/157G/160L/253V/311D/315V/328L/342G/345R/372V/41 iT, 135G/139C/141V/143A/145E/160L/253V/311D/315V/328L/342G/345R/346T/372V, 135G/139C/141V/143A/157K/160L/253V/273T/311D/312Q/315V/328L/342G/345R/372V, 135G/139C/141V/143A/157G/160L/268G/273T/311D/312Q/315V/328L/342G/345R/346T/372V, 135G/141V/143A/157G/160L/273T/311D/312Q/315V/328L/342G/345R/346T/372V, 135G/139C/141V/143A/160L/311D/315V/328L/342G/345R/372V/41 iT, 135G/139C/141V/143A/160L/253V/268G/273F/281C/311D/312I/315V/328L/342G/345R/346T/372V/411T, 135G/141V/143A/157K/160L/273F/311D/315V/328L/342G/345R/346T/372V/411T, 135G/139C/141V/143A/145E/157G/160L/162I/253V/273F/281V/311D/312Q/315V/328L/342G/345R/372V, 135G/139C/141V/143A/160L/253V/273T/281C/311D/312Q/315V/328L/342G/345R/372V, 135G/141V/143A/157G/160L/253V/268F/273F/281V/311D/312Q/315V/328L/342G/345R/372V, 135G/139C/141V/143A/160L/253I/268F/311D/315V/328L/342G/345R/372V, 135G/139C/141V/143A/157R/160L/311D/312Q/315V/328L/342G/345R/372V, 135G/141V/143A/160L/253V/273T/281C/311D/315V/328L/342G/345R/346T/372V, 135G/141V/143A/157K/160L/253V/311D/312I/315V/328L/342G/345R/346T/372V/411T, 135G/141V/143A/157R/160L/273T/311D/312Q/315V/328L/342G/345R/346T/372V/41 IT, 135G/139C/141V/143A/145E/157K/160L/253V/268G/281C/311D/312Q/315V/328L/342G/345R/372V, 135G/139C/141V/143A/160L/273T/311D/312Q/315V/328L/342G/345R/346T/372V, 135G/141V/143A/157K/160L/253V/268F/273T/311D/312I/315V/328L/342G/345R/346T/372V, 135G/139C/141V/143A/160L/268G/311D/315V/328L/342G/345R/346T/372V, 135G/141V/143A/157K/160L/311D/315V/328L/342G/345R/372V, 135G/141V/143A/160L/268G/273F/311D/312Q/315V/328L/342G/345R/346T/372V, 135G/139C/141V/143A/157K/160L/253V/268F/273F/311D/312Q/315V/328L/342G/345R/372V, 135G/141V/143A/157K/160L/273T/311D/312Q/315V/328L/342G/345R/346T/372V, 135G/139C/141V/143A/157G/160L/253V/311D/315V/328L/342G/345R/372V, 135G/139C/141V/143A/160L/311D/315V/328L/342G/345R/372V, 135G/139C/141V/143A/160L/253V/281V/311D/315V/328L/342G/345R/372V, 135G/139C/141V/143A/157G/160L/253V/268G/273T/311D/315V/328L/342G/345R/372V, 135G/141V/143A/160L/253V/311D/312Q/315V/328L/342G/345R/372V/41 IT, 135G/139C/141V/143A/160L/268G/273T/311D/315V/328L/342G/345R/372V, or 135G/141V/143A/160L/253I/311D/315V/328L/342G/345R/372V, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution set at amino acid positions 135/139/141/143/157/160/268/273/311/312/315/31/328/342/345/346/372, 135/139/141/143/157/160/268/273/311/312/315/318/328/342/345/346/372, 135/139/141/143/157/160/268/273/296/311/312/315/328/342/345/346/372, 135/139/141/143/157/160/252/268/273/311/312/315/328/342/345/346/372, 135/139/141/143/157/160/268/273/303/311/312/315/328/342/345/346/372, 135/139/141/143/157/160/253/268/273/311/312/315/328/342/345/346/372, 135/139/141/143/157/160/268/273/311/312/315/328/342/345/346/372/413, 135/139/141/143/157/160/268/273/311/312/315/328/342/345/346/372/386, 135/139/141/143/157/160/268/273/311/312/315/328/342/345/346/372, 135/139/141/143/157/160/235/268/273/311/312/315/328/342/345/346/372, 135/139/141/143/157/160/268/273/311/312/315/328/342/345/346/372/412, 135/139/141/143/157/160/268/273/302/311/312/315/328/342/345/346/372, 135/139/141/143/157/160/268/273/311/312/315/328/342/345/346/371/372, 135/139/141/143/157/160/268/273/311/312/315/328/342/345/346/372/405, 135/139/141/143/157/160/268/273/311/312/315/328/342/345/346/372/389, 135/139/141/143/157/160/268/273/311/312/315/328/342/345/346/372/391, 135/139/141/143/157/160/268/273/311/312/315/328/342/345/346/358/372, 135/141/143/157/160/268/312/315/342/345/346, 135/139/141/143/157/160/268/273/312/315/328/342/345/346/372, 135/139/141/143/157/160/268/273/311/315/328/342/345/346/372, 135/139/141/143/157/160/268/273/311/312/315/328/342/345/346, 135/141/312/328/342/345/346/372, 135/139/141/157/160/268/273/311/312/315/328/342/345/346/372, 135/141/157/160/268/273/311/312/315/328/342/345/346/372, 135/141/143/157/268/273/311315/328/342/345/346, 135/139/141/157/160/268/311/312/315/342/345/346/372, 135/139/141/143/157/160/268/273/311/312/315/328/342/345/372, 141/143/157/273/311/315/328/345/372, 135/143/157/160/268/311/312/315/328/342/345/346/372, 139/157/160/311/315/328/342/345/346, 135/157/160/268/273/312/315/328/342/345/346/372, 135/141/143/160/273/311/312/315/342/345, 53/135/157/160/268/311/312/315/328/342/345/346, 135/141/143/157/160/268/273/311/312/315/328/342/345/346/372, 135/157/160/268/311/315/328/342/345/346/372, 135/137/141/143/157/160/221/233/268/273/311/312/315/328/342/345/346/372/413, 135/139/141/143/157/160/233/268/273/311/312/315/328/342/345/346/372, 135/139/141/143/157/160/221/268/273/279/311/312/315/328/342/345/346/372, 135/137/141/143/157/160/233/268/273/279/311/312/315/328/342/345/346/372, 135/139/141/143/157/160/221/268/273/311/312/315/328/342/345/346/372, 135/139/141/143/157/160/214/268/273/311/312/315/328/342/345/346/372, 135/137/141/143/156/157/160/268/273/311/312/315/328/342/345/346/372, 135/139/141/143/157/160/214/221/268/273/311/312/315/328/342/345/346/372, 135/137/139/141/143/157/160/221/233/268/273/279/311/312/315/328/342/345/346/372, 135/137/139/141/143/157/160/221/268/273/311/312/315/328/342/345/346/372, 135/137/139/141/143/157/160/268/273/311/312/315/328/342/345/346/372, 135/137/139/141/143/157/160/268/273/279/311/312/315/328/342/345/346/372, 135/137/141/143/157/160/268/273/311/312/315/328/342/345/346/372, 135/137/139/141/143/157/160/214/268/273/279/311/312/315/328/342/345/346/372, 135/139/141/143/157/160/266/268/273/311/312/315/328/342/345/346/372, 135/137/139/141/143/156/157/160/214/268/273/311/312/315/328/342/345/346/372, 135/137/139/141/143/157/160/221/233/268/273/311/312/315/328/342/345/346/372, 135/137/141/143/156/157/160/221/268/273/311/312/315/328/342/345/346/372, 135/137/139/141/143/157/160/214/268/273/311/312/315/328/342/345/346/372, 135/139/141/143/157/160/268/273/279/311/312/315/328/342/345/346/372, 135/137/139/141/143/157/160/233/268/273/311/312/315/328/342/345/346/372, 135/137/141/143/157/160/214/233/268/273/311/312/315/328/342/345/346/372, 135/139/141/143/157/160/221/268/273/311/312/315/328/342/345/346/372/413, 135/137/139/141/143/157/160/214/233/268/273/311/312/315/328/342/345/346/372, 135/137/141/143/157/160/221/233/268/273/279/311/312/315/328/342/345/346/372, 135/137/139/141/143/156/157/160/268/273/311/312/315/328/342/345/346/372, 135/137/139/141/143/157/160/221/268/273/311/312/315/328/342/345/346/372/413, 135/137/139/141/143/157/160/268/273/311/312/315/328/342/345/346/372/413, 135/137/139/141/143/157/160/221/268/273/279/311/312/315/328/342/345/346/372, or 135/137/141/143/156/157/160/214/233/268/273/311/312/315/328/342/345/346/372/413, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution set or amino acid residues 31G/135G/139C/141V/143A/157G/160L/268G/273T/311D/312Q/315V/328L/342G/345R/346T/372V, 135G/139C/141V/143A/157G/160L/268G/273T/311D/312Q/315V/318R/328L/342G/345R/346T/372V, 135G/139C/141V/143A/157G/160L/268G/273T/296R/311D/312Q/315V/328L/342G/345R/346T/372V, 135G/139C/141V/143A/157G/160L/268G/273T/296M/311D/312Q/315V/328L/342G/345R/346T/372V, 135G/139C/141V/143A/157G/160L/252P/268G/273T/311D/312Q/315V/328L/342G/345R/346T/372V, 135G/139C/141V/143A/157G/160L/268G/273T/303A/311D/312Q/315V/328L/342G/345R/346T/372V, 135G/139C/141V/143A/157G/160L/253C/268G/273T/311D/312Q/315V/328L/342G/345R/346T/372V, 135G/139C/141V/143A/157G/160L/268G/273T/311D/312Q/315V/328L/342G/345R/346T/372V/413C, 135G/139C/141V/143A/157G/160L/268G/273T/311D/312Q/315V/328L/342G/345R/346T/372V/386P, 135G/139C/141V/143A/157G/160L/268G/273T/311D/312Q/315V/328L/342G/345R/346T/372V/413A, 135G/139C/141V/143A/157G/160L/268G/273T/311D/312R/315V/328L/342G/345R/346T/372V, 135G/139C/141V/143A/157G/160L/235R/268G/273T/311D/312Q/315V/328L/342G/345R/346T/372V, 135G/139C/141V/143A/157G/160L/268G/273T/311D/312Q/315V/328L/342G/345R/346T/372V/412P, 135G/139C/141V/143A/157G/160L/268G/273T/311D/312Q/315V/328L/342A/345R/346T/372V, 135G/139C/141V/143A/157G/160L/268G/273T/311D/312Q/315V/328L/342G/345R/346T/372V/413S, 135G/139C/141V/143A/157G/160L/268G/273T/302P/311D/312Q/315V/328L/342G/345R/346T/372V, 135G/139C/141V/143A/157G/160L/268G/273T/311D/312Q/315V/328L/342G/345R/346T/371L/372V, 135G/139C/141V/143A/157G/160L/268G/273T/311D/312Q/315V/328L/342G/345R/346T/372V/405L, 135G/139C/141V/143A/157G/160L/268G/273T/311D/312A/315V/328L/342G/345R/346T/372V, 135G/139C/141V/143A/157G/160L/268G/273T/311D/312Q/315V/328L/342G/345R/346T/372V/389P, 135G/139C/141V/143A/157G/160L/268G/273T/311D/312Q/315V/318P/328L/342G/345R/346T/372V, 135G/139C/141V/143A/157G/160L/268G/273T/311D/312Q/315V/328L/342G/345R/346T/372V/391S, 135G/139C/141V/143A/157G/160L/268G/273T/311D/312Q/315V/328L/342G/345R/346T/372V/412T, 135G/139C/141V/143A/157G/160L/268G/273T/311D/312Q/315V/328L/342G/345R/346T/358S/372V, 135G/139C/141V/143A/157G/160L/268G/273T/311D/312Q/315V/328L/342G/345R/346T/372V/389C, 135G/139C/141V/143A/157G/160L/235V/268G/273T/311D/312Q/315V/328L/342G/345R/346T/372V, 135G/139C/141V/143A/157G/160L/268G/273T/311D/312Q/315V/328L/342G/345R/346T/372V/391L, 135G/141V/143A/157G/160L/268G/312Q/315V/342G/345R/346T, 135G/139C/141V/143A/157G/160L/268G/273T/312Q/315V/328L/342G/345R/346T/372V, 135G/139C/141V/143A/157G/160L/268G/273T/311D/315V/328L/342G/345R/346T/372V, 135G/139C/141V/143A/157G/160L/268G/273T/311D/312Q/315V/328L/342G/345R/346T, 135G/141V/312Q/328L/342G/345R/346T/372V, 135G/139C/141V/157G/160L/268G/273T/311D/312Q/315V/328L/342G/345R/346T/372V, 135G/141V/157G/160L/268G/273T/311D/312Q/315V/328L/342G/345R/346T/372V, 135G/141V/143A/157G/268G/273T/311D/315V/328L/342G/345R/346T, 135G/139C/141V/157G/160L/268G/311D/312Q/315V/342G/345R/346T/372V, 135G/139C/141V/143A/157G/160L/268G/273T/311D/312Q/315V/328L/342G/345R/372V, 141V/143A/157G/273T/311D/315V/328L/345R/372V, 135G/143A/157G/160L/268G/311D/312Q/315V/328L/342G/345R/346T/372V, 139C/157G/160L/311D/315V/328L/342G/345R/346T, 135G/157G/160L/268G/273T/312Q/315V/328L/342G/345R/346T/372V, 135G/141V/143A/160L/273T/311D/312Q/315V/342G/345R, 53A/135G/157G/160L/268G/311D/312Q/315V/328L/342G/345R/346T, 135G/141V/143A/157G/160L/268G/273T/311D/312Q/315V/328L/342G/345R/346T/372V, 135G/157G/160L/268G/311D/315V/328L/342G/345R/346T/372V, 135G/139L/141V/143A/157G/160L/268G/273T/311D/312Q/315V/328L/342G/345R/346T/372V, 135G/137A/141V/143A/157G/160L/221Q/233L/268G/273T/311D/312Q/315V/328L/342G/345R/346T/372V/413D, 135G/139C/141V/143A/157G/160L/S233L/268G/273T/311D/312Q/315V/328L/342G/345R/346T/372V 135G/139C/141V/143A/157G/160L/221Q/268G/273T/279K/311D/312Q/315V/328L/342G/345R/346T/372V, 135G/137N/141V/143A/157G/160L/233L/268G/273T/279M/311D/312Q/315V/328L/342G/345R/346T/372V, 135G/139C/141V/143A/157G/160L/221Q/268G/273T/311D/312Q/315V/328L/342G/345R/346T/372V, 135G/139L/141V/143A/157G/160L/214V/268G/273T/311D/312Q/315V/328L/342G/345R/346T/372V, 135G/137N/141V/143A/T156V/157G/160L/268G/273T/311D/312Q/315V/328L/342G/345R/346T/372V, 135G/139C/141V/143A/157G/160L/268G/273T/311D/312Q/315V/328L/342G/345R/346T/372V, 135G/139L/141V/143A/157G/160L/214V/221Q/268G/273T/311D/312Q/315V/328L/342G/345R/346T/372V, 135G/137A/139L/141V/143A/157G/160L/221Q/233L/268G/273T/279K/311D/312Q/315V/328L/342G/345R/346T/372V, 135G/137A/139L/141V/143A/157G/160L/221Q/268G/273T/311D/312Q/315V/328L/342G/345R/346T/372V, 135G/137N/139L/141V/143A/157G/160L/268G/273T/311D/312Q/315V/328L/342G/345R/346T/372V, 135G/137N/139L/141V/143A/157G/160L/268G/273T/279K/311D/312Q/315V/328L/342G/345R/346T/372V, 135G/137A/141V/143A/157G/160L/268G/273T/311D/312Q/315V/328L/342G/345R/346T/372V, 135G/137N/139C/141V/143A/157G/160L/268G/273T/311D/312Q/315V/328L/342G/345R/346T/372V, 135G/137N/139L/141V/143A/157G/160L/221Q/268G/273T/311D/312Q/315V/328L/342G/345R/346T/372V, 135G/137N/139L/141V/143A/157G/160L/214P/268G/273T/279M/311D/312Q/315V/328L/342G/345R/346T/372V, 135G/139C/141V/143A/157G/160L/266Y/268G/273T/311D/312Q/315V/328L/342G/345R/346T/372V, 135G/139L/141V/143A/157G/160L/221Q/268G/273T/311D/312Q/315V/328L/342G/345R/346T/372V, 135G/137N/139C/141V/143A/157G/160L/221Q/268G/273T/311D/312Q/315V/328L/342G/345R/346T/372V, 135G/137N/141V/143A/157G/160L/268G/273T/311D/312Q/315V/328L/342G/345R/346T/372V, 135G/137N/139C/141V/143A/156V/157G/160L/214V/268G/273T/311D/312C/315V/328L/342G/345R/346T/372V, 135G/137N/139C/141V/143A/157G/160L/221Q/233L/268G/273T/311D/312Q/315V/328L/342G/345R/346T/372V, 135G/137N/141V/143A/156V/157G/160L/221Q/268G/273T/311D/312Q/315V/328L/342G/345R/346T/372V, 135G/137A/139L/141V/143A/157G/160L/214V/268G/273T/311D/312Q/315V/328L/342G/345R/346T/372V, 135G/139C/141V/143A/157G/160L/268G/273T/311D/312Q/315V/328L/342G/345R/346T/372V/413D, 135G/139C/141V/143A/157G/160L/268G/273T/279K/311D/312Q/315V/328L/342G/345R/346T/372V, 135G/139C/141V/143A/157G/160L/268G/273T/279M/311D/312Q/315V/328L/342G/345R/346T/372V, 135G/137A/139L/141V/143A/157G/160L/233L/268G/273T/311D/312Q/315V/328L/342G/345R/346T/372V, 135G/137N/141V/143A/157G/160L/214V/233I/268G/273T/311D/312Q/315V/328L/342G/345R/346T/372V, 135G/139C/141V/143A/157G/160L/221Q/268G/273T/311D/312Q/315V/328L/342G/345R/346T/372V/413D, 135G/137N/139C/141V/143A/157G/160L/S214V/S233L/268G/273T/311D/312Q/315V/328L/342G/345R/346T/372V, 135G/137A/139L/141V/143A/157G/160L/214P/268G/273T/311D/312Q/315V/328L/342G/345R/346T/372V, 135G/137N/141V/143A/157G/160L/221Q/233L/268G/273T/Q279M/311D/312Q/315V/328L/342G/345R/346T/372V, 135G/137N/139C/141V/143A/156V/157G/160L/268G/273T/311D/312Q/315V/328L/342G/345R/346T/372V, 135G/137N/139L/141V/143A/157G/160L/221Q/233I/268G/273T/311D/312Q/315V/328L/342G/345R/346T/372V, 135G/139C/141V/143A/157G/160L/214V/221Q/268G/273T/311D/312Q/315V/328L/342G/345R/346T/372V, 135G/137N/139C/141V/143A/157G/160L/221Q/268G/273T/311D/312Q/315V/328L/342G/345R/346T/372V/413D, 135G/137A/139L/141V/143A/157G/160L/221Q/233L/268G/273T/279M/311D/312Q/315V/328L/342G/345R/346T/372V, 135G/139C/141V/143A/157G/160L/214V/268G/273T/311D/312Q/315V/328L/342G/345R/346T/372V, 135G/137A/139L/141V/143A/157G/160L/268G/273T/311D/312Q/315V/328L/342G/345R/346T/372V, 135G/137N/139C/141V/143A/157G/160L/S233L/268G/273T/311D/312Q/315V/328L/342G/345R/346T/372V, 135G/137A/139C/141V/143A/157G/160L/268G/273T/311D/312Q/315V/328L/342G/345R/346T/372V, 135G/137N/139C/141V/143A/157G/160L/268G/273T/311D/312Q/315V/328L/342G/345R/346T/372V/413D, 135G/137N/139C/141V/143A/157G/160L/221Q/268G/273T/279K/311D/312Q/315V/328L/342G/345R/346T/372V, 135G/137N/141V/143A/T156V/157G/160L/214V/233L/268G/273T/311D/312Q/315V/328L/342G/345R/346T/372V/413D, or 135G/137N/139L/141V/143A/156V/157G/160L/214V/268G/273T/311D/312Q/315V/328L/342G/345R/346T/372V, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution set at amino acid positions 135/137/139/141/143/145/157/160/214/268/273/279/311/312/315/328/342/345/346, 135/137/139/141/143/145/157/160/214/256/268/273/279/311/312/315/328/342/345/346/372, 135/137/139/141/143/157/160/214/221/243/268/273/279/311/312/315/342/345/346, 135/137/139/141/143/157/160/214/268/273/279/311/312/315/328/342/345/346, 135/137/139/141/143/145/157/160/214/221/268/273/279/311/312/315/328/342/345/346/372/406, 135/137/139/141/143/157/160/214/268/273/279/311/312/315/342/345/346/372, 135/137/139/141/143/145/157/160/169/214/268/273/279/311/312/315/328/342/345/372/406, 135/137/139/141/143/157/160/214/256/268/273/279/311/312/315/328/342/345/346/372, 135/137/139/141/143/145/157/160/214/221/268/273/279/311/312/315/342/345/372/406, 135/137/139/141/143/157/160/214/243/268/273/279/311/312/315/328/342/345/346/372, 135/137/139/141/143/145/157/160/214/256/268/273/279/311/312/315/342/345/346, 135/137/139/141/143/157/160/169/214/221/268/273/279/311/312/315/342/345/346/406, 135/137/139/141/143/157/160/214/268/273/279/311/312/315/328/342/345/346/406, 135/137/139/141/143/157/160/169/214/268/273/279/311/312/315/342/345/346/372/406, 135/137/139/141/143/157/160/214/221/268/273/279/311/312/315/328/342/345/346, 135/137/139/141/143/145/157/160/214/221/268/273/279/311/312/315/342/345/346, 135/137/139/141/143/157/160/214/243/268/273/279/311/312/315/342/345/346/372, 135/137/139/141/143/145/157/160/214/268/273/279/311/312/315/328/342/345/346/372, 135/137/139/141/143/157/160/214/256/268/273/279/311/312/315/328/342/345, 135/137/139/141/143/157/160/214/221/268/273/279/311/312/315/328/342/345/346/372/406, 135/137/139/141/143/157/160/169/214/268/273/279/311/312/315/328/342/345/346, 135/137/139/141/143/145/157/160/214/221/268/273/279/311/312/315/328/342/345/346/372, 135/137/139/141/143/145/157/160/214/221/268/273/279/311/312/315/328/342/345, 135/137/139/141/143/157/160/214/243/268/273/279/311/312/315/342/345/346/406, 135/137/139/141/143/145/157/160/169/214/268/273/279/311/312/315/342/345/372, 135/137/139/141/143/145/157/160/214/221/268/273/279/311/312/315/342/345/346/372, 135/137/139/141/143/157/160/169/214/268/273/279/311/312/315/328/342/345/346/372, 135/137/139/141/143/157/160/214/221/268/273/279/311/312/315/342/345/346/372, 135/137/139/141/143/157/160/214/221/268/273/279/311/312/315/328/342/345/346/372, 135/137/139/141/143/145/157/160/214/268/273/279/311/312/315/342/345/346/372, 135/137/139/141/143/157/160/214/268/273/279/311/312/315/328/342/345/372, 135/137/139/141/143/157/160/214/268/273/279/311/312/315/328/342/345/346/372, 135/137/139/141/143/157/160/212/214/268/273/279/311/312/315/328/342/345/346/372, 135/137/139/141/143/145/157/160/214/268/273/279/311/312/315/328/342/345/346/372, 135/137/139/141/143/157/160/179/214/268/273/279/311/312/315/328/342/345/372, 135/137/139/141/143/157/160/214/268/273/279/311/312/315/328/342/345/346/372/375, 135/137/139/141/143/157/160/214/264/268/273/279/311/312/315/328/342/345/346/372, 135/137/139/141/143/157/160/179/214/268/273/279/311/312/315/328/342/345/346/372, 135/137/139/141/143/157/160/185/214/268/273/279/311/312/315/328/342/345/346/372, 135/137/139/141/143/157/160/214/268/273/279/311/312/315/328/342/345/346/372, 135/137/139/141/143/157/160/214/220/268/273/279/311/312/315/328/342/345/346/372, or 135/137/139/141/143/157/160/214/268/273/279/311/312/315/324/328/342/345/346/372, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution set or amino acid residue 135G/137N/139L/141V/143A/145E/157G/160L/214P/268G/273L/279M/311D/312Q/315V/328L/342G/T345R/346T, 135G/137N/139L/141V/143A/145E/157G/160L/214P/256M/268G/273L/279M/311D/312Q/315V/328L/342G/345R/346T/372V, 135G/137N/139L/141V/143A/157G/160L/214P/221Q/243L/268G/273L/279M/311D/312Q/315V/342G/345R/346T, 135G/137N/139L/141V/143A/157G/160L/214P/268G/273T/279M/311D/312Q/315V/328L/342G/345R/346T, 135G/137N/139L/141V/143A/145E/157G/160L/214P/221Q/268G/273T/279L/311D/312Q/315V/328L/342G/345R/346T/372V/406R, 135G/137N/139L/141V/143A/157G/160L/214P/268G/273L/279M/311D/312Q/315V/342G/345R/346T/372V, 135G/137N/139L/141V/143A/145E/157G/160L/169S/214P/268G/273L/279M/311D/312Q/315V/328L/342G/345R/372V/406R, 135G/137N/139L/141V/143A/157G/160L/214P/256M/268G/273L/279M/311D/312Q/315V/328L/342G/345R/346T/372V, 135G/137N/139L/141V/143A/145E/157G/160L/214P/221Q/268G/273L/279M/311D/312Q/315V/342G/345R/372V/406R, 135G/137N/139L/141V/143A/157G/160L/214P/243L/268G/273L/279M/311D/312Q/315V/328L/342G/345R/346T/372V, 135G/137N/139L/141V/143A/145E/157G/160L/214V/256M/268G/273L/279L/311D/312Q/315V/342G/345R/346T, 135G/137N/139L/141V/143A/157G/160L/T169S/214P/221Q/268G/273T/279M/311D/312Q/315V/342G/345R/346T/406R, 135G/137N/139L/141V/143A/157G/160L/214P/268G/273T/279M/311D/312Q/315V/328L/342G/345R/346T/406R, 135G/137N/139L/141V/143A/157G/160L/169S/214P/268G/273T/279M/311D/312Q/315V/342G/345R/346T/372V/406R, 135G/137N/139L/141V/143A/157G/160L/214P/221Q/268G/273T/279M/311D/312Q/315V/328L/342G/345R/346T, 135G/137N/139L/141V/143A/145E/157G/160L/214P/221Q/268G/273T/279L/311D/312Q/315V/342G/345R/346T, 135G/137N/139L/141V/143A/157G/160L/214V/243L/268G/273L/279M/311D/312Q/315V/342G/345R/346T/372V, 135G/137N/139L/141V/143A/145E/157G/160L/214P/221Q/268G/273T/279M/311D/312Q/315V/328L/342G/345R/346T/372V, 135G/137N/139L/141V/143A/157G/160L/214V/256M/268G/273L/279M/311D/312Q/315V/328L/342G/345R, 135G/137N/139L/141V/143A/157G/160L/214P/221Q/268G/273T/279M/311D/312Q/315V/328L/342G/345R/346T/372V/406R, 135G/137N/139L/141V/143A/157G/160L/T169S/214P/268G/273T/279M/311D/312Q/315V/328L/342G/345R/346T, 135G/137N/139L/141V/143A/145E/157G/160L/214V/221Q/268G/273L/279M/311D/312Q/315V/328L/342G/345R/346T/372V, 135G/137N/139L/141V/143A/145E/157G/160L/214P/221Q/268G/273T/279M/311D/312Q/315V/328L/342G/345R, 135G/137N/139L/141V/143A/157G/160L/214P/243L/268G/273L/279M/311D/312Q/315V/342G/345R/346T/406R, 135G/137N/139L/141V/143A/145E/157G/160L/169S/214P/268G/273L/279M/311D/312Q/315V/342G/345R/372V, 135G/137N/139L/141V/143A/157G/160L/214P/268G/273T/279M/311D/312Q/315V/342G/345R/346T/372V, 135G/137N/139L/141V/143A/157G/160L/169S/214P/268G/273L/279M/311D/312Q/315V/328L/342G/345R/346T, 135G/137N/139L/141V/143A/145E/157G/160L/214P/221Q/268G/273T/279M/311D/312Q/315V/342G/345R/346T/372V, 135G/137N/139L/141V/143A/157G/160L/169S/214V/268G/273L/279M/311D/312Q/315V/328L/342G/345R/346T/372V, 135G/137N/139L/141V/143A/157G/160L/214P/221Q/268G/273L/279M/311D/312Q/315V/342G/345R/346T/372V, 135G/137N/139L/141V/143A/157G/160L/214P/221Q/268G/273T/279M/311D/312Q/315V/328L/342G/345R/346T/372V, 135G/137N/139L/141V/143A/145E/157G/160L/214P/268G/273T/279M/311D/312Q/315V/342G/345R/346T/372V, 135G/137N/139L/141V/143A/157G/160L/214N/268G/273T/279M/311D/312Q/315V/328L/342G/345R/372V, 135G/137N/139L/141V/143A/157G/160L/214P/268G/273T/279M/311D/312R/315V/328L/342G/345R/346T/372V, 135G/137N/139L/141V/143A/157G/160L/212S/214P/268G/273T/279M/311D/312Q/315V/328L/342G/345R/346T/372V, 135G/137N/139L/141V/143A/157G/160L/214P/268G/273T/279K/311D/312Q/315V/328L/342G/345R/346T/372V, 135G/137N/139L/141V/143A/157G/160L/212S/214P/268G/273T/279M/311D/312R/315V/328L/342G/345R/346T/372V, 135G/137N/139L/141V/143A/145E/157G/160L/214P/268G/273T/279M/311D/312Q/315V/328L/342G/345R/346T/372V, 135G/137N/139L/141V/143A/157G/160L/179S/214P/268G/273T/279M/311D/312Q/315V/328L/342G/345R/372V, 135G/137N/139L/141V/143A/157G/160L/214N/268G/273T/279M/311D/312Q/315V/328L/342G/345R/346T/372V, 135G/137N/139L/141V/143A/157G/160L/214P/268G/273T/279M/311D/312Q/315V/328L/342G/345R/372V, 135G/137N/139L/141V/143A/157G/160L/214P/268G/273T/279M/311D/312Q/315R/328L/342G/345R/346T/372Y, 135G/137N/139L/141V/143A/157G/160L/214P/268G/273T/279M/311D/312Q/315V/328L/342G/345R/346T/372Y, 135G/137N/139L/141V/143A/157G/160L/214P/268G/273T/279M/311D/312Q/315V/328L/342G/345R/346T/372V/375A, 135G/137N/139L/141V/143A/157G/160L/214P/264F/268G/273T/279M/311D/312Q/315V/328L/342G/345R/346T/372V, 135G/137N/139L/141V/143A/157G/160L/179S/214P/268G/273T/279M/311D/312Q/315V/328L/342G/345R/346T/372V, 135G/137N/139L/141V/143A/157G/160L/185T/214P/268G/273T/279M/311D/312Q/315V/328L/342G/345R/346T/372V, 135G/137N/139L/141V/143A/157G/160L/214P/268G/273T/279M/311D/312Q/315V/328L/342G/345R/346T/372V, 135G/137N/139L/141V/143A/157G/160L/214P/220R/268G/273T/279M/311D/312Q/315V/328L/342G/345R/346T/372Y, 135G/137N/139L/141V/143A/157G/160L/214P/268G/273T/279M/311D/312Q/315V/324D/328L/342G/345R/346T/372V, or 135G/137N/139L/141V/143A/145E/157G/160L/214P/221Q/268G/273L/279M/311D/312Q/315V/342G/345R/346T, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution at an amino acid position set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or substitution set at amino acid position(s) set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the amino acid sequence of the engineered protease polypeptide comprises at least a substitution or substitution set of an engineered protease polypeptide variant set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to an amino acid sequence comprising at least a substitution or substitution set of an engineered protease polypeptide set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 948, 1126, 1368, or 1548, or to the reference sequence corresponding to SEQ ID NO: 948, 1126, 1368, or 1548.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 6-2242, or to a reference sequence corresponding to an even-numbered SEQ ID NO. of SEQ ID NOs: 6-2242.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of an even-numbered SEQ ID NO. of SEQ ID NOs: 6-2242, or to the reference sequence corresponding to an even-numbered SEQ ID NO. of SEQ ID NOs: 6-2242, wherein the amino acid sequence comprises one or more substitutions relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 948, 1126, 1368, or 1548, or to the reference sequence corresponding to SEQ ID NO: 948, 1126, 1368, or 1548, wherein the amino acid sequence comprises one or more substitutions relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the amino acid sequence of the engineered protease polypeptide comprises at least a substitution at amino acid position 11, 31, 42, 45, 50, 53, 84, 99, 100, 126, 128, 129, 130, 131, 132, 134, 135, 136, 137, 138, 139, 140, 141, 143, 145, 151, 154, 156, 157, 159, 160, 161, 162, 163, 169, 172, 173, 174, 179, 180, 184, 185, 186, 187, 188, 190, 191, 192, 193, 194, 198, 199, 212, 214, 220, 221, 222, 223, 225, 231, 232, 233, 235, 237, 238, 239, 240, 242, 243, 245, 246, 249, 250, 251, 252, 253, 254, 256, 258, 262, 263, 264, 265, 266, 267, 268, 269, 271, 273, 274, 275, 277, 278, 279, 280, 281, 283, 285, 290, 292, 293, 294, 296, 297, 300, 302, 303, 311, 312, 313, 314, 315, 316, 318, 324, 328, 336, 339, 341, 342, 343, 345, 346, 355, 358, 360, 364, 367, 368, 369, 370, 371, 372, 373, 374, 375, 377, 381, 382, 384, 386, 389, 391, 392, 401, 402, 405, 406, 409, 410, 411, 412, or 413, or combinations thereof, wherein the amino acid positions are relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the amino acid sequence of the engineered protease polypeptide comprises at least a substitution or amino acid residue 11K, 31G, 42W, 45Y, 50R, 53A, 84M, 99V, 100V, 126T, 128G/I/K/L/P/R/S/T/V, 129E/F/H/I/K/L/R/S/T/V, 130A/F/G/N/V, 131E/P/R/T/V/Y, 132A/C/D/E/G/P/R/V/Y, 134A/C/D/E/G/I/L/M/N/P/S/T/V/W/Y, 135A/C/E/G/H/I/K/L/M/N/P/R/S/T/V/W/Y, 136C/G/I/M, 137A/D/N/S, 138Q, 139C/D/E/F/H/I/K/L/M/N/R/S, 140L, 141A/C/D/E/F/G/H/I/L/M/N/Q/R/S/T/V/W/Y, 143A/C/D/H/N/Q/S/T, 145A/C/D/E/F/G/H/I/K/L/P/Q/R/S/T/V/W, 151D/Q, 154C/D/L/R, 156C/V, 157A/C/D/E/F/G/H/I/K/L/M/N/P/Q/R/S/T/V/W, 159G, 160A/C/D/E/F/K/L/M/N/P/R/Q/S/T/V/W/Y, 161D/E/G/L/R, 162I, 163H/L, 169S, 172Q, 173F/S, 174L, 179K/S, 180H/L/M, 184A/D/G/L/M/Q/R, 185A/D/E/F/G/L/M/P/Q/R/S/T/V, 186A/R/S/T/Y, 187A, 188A/C/D/F/G/L/M/S/T/W, 190S, 191R, 192C/D/M/N, 193T, 194A/D/L/T, 198G, 199C/K/L, 212S, 214A/C/D/E/F/G/H/I/K/L/M/N/P/Q/R/T/V/W/Y, 220K/L/R, 221A/C/D/E/F/G/H/I/K/L/M/P/Q/R/T/V/W/Y, 222G, 223S, 225V, 231H/V, 232S, 233G/I/L, 235Q/R/V, 237A/G, 238Q, 239L/M, 240A/L, 242E/S, 243E/L/M/R/S/T, 245L/V, 246I/V, 249G/M/S, 250A/C/F/L/N/T, 251D/S/T, 252P, 253C/I/V, 254C/E, 256L/M, 258W, 262A/S, 263E/H/P/Q/R/S, 264A/C/F/I/L/N/P/R/T/V, 265C/G/R, 266H/T/Y, 267A/G/H/I/L/M/R/S/T/V/W, 268A/F/G/H/I/N/P/Q/S/T/V/Y, 269Q/T, 271A, 273A/C/F/L/M/S/T/V, 274A/G/K/L/T/V/W, 275A/V, 277D/G, 278L/N/S/V/Y, 279A/E/F/G/H/K/L/M/R/S/T/Y/V/W, 280D/K/S/T, 281C/V, 283M, 285S, 290E/G/S, 292V, 293A, 294V/W, 296M/R, 297F, 300R/V, 302G/P, 303A/V, 311A/E/D/G/K/M/Q/S/T, 312A/C/D/E/G/H/I/K/L/M/N/P/Q/R/S/T/V/W/Y, 313A/Q/S/T, 314G, 315A/C/D/E/F/G/H/I/K/L/M/Q/R/S/T/V/W/Y, 316K, 318N/P/R, 324A/D/E/I/R/V/W/Y, 328L/M/V, 336F, 339S/W, 341G, 342A/C/D/E/F/G/I/K/M/N/P/R/Q/S/T/V/W/Y, 343S, 345A/C/D/E/F/G/I/K/L/M/Q/R/S/V/W/Y, 346A/C/D/F/G/K/L/M/N/P/Q/R/S/T/V/W/Y, 355A, 358S, 360S, 364A/V, 367V, 368G/T, 369I/V/W, 370C/E/F/G/I/K/L/P/Q/R/S/V, 371L, 372A/C/F/L/R/S/V/Y, 373A/C/E/F/M/S/Y, 374E/G/L/R/S/W/Y, 375A/E/I/L/M/S/T/V, 377H, 381N, 382G/R/S/T, 384C, 386P/W, 389C/P, 391L/S, 392Y, 401L, 402G/*, 405L/Q, 406C/M/R/W, 409E/R/*, 410C/I/W/*, 411L/R/T/V, 412P/T/*, or 413A/C/D/S/*, or combinations thereof, wherein the amino acid positions are relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the amino acid sequence of the engineered protease polypeptide comprises at least a substitution at amino acid position 135, 137, 139, 141, 143, 157, 160, 214, 268, 273, 279, 311, 312, 315, 328, 342, 345, 346, or 372, or combinations thereof, wherein the amino acid positions are relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the amino acid sequence of the engineered protease polypeptide comprises at least a substitution or amino acid residue 135A/C/E/G/H/I/K/L/M/N/P/R/S/T/V/W/Y, 137A/D/N/S, 139C/D/E/F/H/I/K/L/M/N/R/S, 141A/C/D/E/F/G/H/I/L/M/N/Q/R/S/T/V/W/Y, 143A/C/D/H/N/Q/S/T, 157A/C/D/E/F/G/H/I/K/L/M/N/P/Q/R/S/T/V/W, 160A/C/D/E/F/K/L/M/N/P/R/Q/S/T/V/W/Y, 214A/C/D/E/F/G/H/I/K/L/M/N/P/Q/R/T/V/W/Y, 268A/F/G/H/I/N/P/Q/S/T/V/Y, 273A/C/F/L/M/S/T/V, 279A/E/F/G/H/K/L/M/R/S/T/Y/V/W, 311A/E/D/G/K/M/Q/S/T, 312A/C/D/E/G/H/I/K/L/M/N/P/Q/R/S/T/V/W/Y, 315A/C/D/E/F/G/H/I/K/L/M/Q/R/S/T/V/W/Y, 328L/M/V, 342A/C/D/E/F/G/I/K/M/N/P/R/Q/S/T/V/W/Y, 345A/C/D/E/F/G/I/K/L/M/Q/R/S/V/W/Y, 346A/C/D/F/G/K/L/M/N/P/Q/R/S/T/V/W/Y, or 372A/C/F/L/R/S/V/Y, or combinations thereof, wherein the amino acid positions are relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the amino acid sequence of the engineered protease polypeptide comprises at least a substitution or amino acid residue 135G, 137N, 139L/C, 141V, 143A, 157G, 160L, 214P, 268G, 273T, 279M, 311D, 312Q, 315V, 328L, 342G, 345R, 346T, or 372V, or combinations thereof, wherein the amino acid positions are relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 948, or to the reference sequence corresponding to SEQ ID NO: 948, wherein the amino acid sequence comprises one or more substitutions relative to the reference sequence corresponding to residues 135-413 of SEQ ID NO: 948, or relative to the reference sequence corresponding to SEQ ID NO: 948.

In some embodiments, the engineered protease polypeptide of comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 950-1154, or to a reference sequence corresponding to an even-numbered SEQ ID NO. of SEQ ID NOs: 950-1154, wherein the amino acid sequence comprises one or more substitutions relative to the reference sequence corresponding to residues 135-413 of SEQ ID NO: 948, or relative to the reference sequence corresponding to SEQ ID NO: 948.

In some embodiments, the amino acid sequence of the engineered protease polypeptide comprises at least a substitution or substitution set at amino acid position(s) 411, 402, 285, 245, 266, 355, 258, 222, 140, 268, 225, 283, 406, 410, 143/145/243/312, 139/143/145/157/312, 139/157/345, 139/269, 156/157/342/346, 139/143, 269, 139/243/328, 269/328, 143/145/169, 328, 143/145/262, 145/262/312/328, 139/156/157, 139/145/312, 312, 139, 139/312, 139/156, 139/143/145/243, 145/157, 145/346, 145/262/312/328/345/346, 145/262, 312/342, 143/243, 139/345, 342, 143/145/262/342, 139/143/169, 139/143/145/312, 169, 139/145/262/312/328/342/345/346, 139/328, 139/243, 139/143/328, 139/143/243, 139/145, 145/312, 145/169, 139/143/157/312, 84/139/143, 145/269, 143/145/157/269/312/328, 143/145/269, 157, 139/143/312, 256, 273, 409, 172, 401, 281, 253, 143/145/243/328, 145, 139/143/145/328/342/345, 143/328/342/345, 145/342/345, 143, 139/145/328/342/345, 143/145/169/312/328/345/346, 143/243/328/342/345/346, 139/143/157/169/328/346, 143/145/156/312/328, 139/145/157/312/328, 143/328/342/345/346, 143/145, 143/145/312/342/345, or 143/145/328, wherein the amino acid positions are relative to the reference sequence corresponding to residues 135-413 of SEQ ID NO: 948, or relative to the reference sequence corresponding to SEQ ID NO: 948.

In some embodiments, the amino acid sequence of the engineered protease polypeptide comprises at least a substitution or substitution set, or amino acid residue(s) 411V, 411R, 402G, 285S, 245V, 266H, 355A, 258W, 222G, 140L, 268T, 411L, 225V, 245L, 283M, 406W, 410C, 406C, 143A/145E/243L/312I, 139L/143A/145E/157G/312I, 139C/157G/345R, 139C/269Q, 156V/157G/342G/346T, 139L/143A, 269Q, 139L/243L/328L, 269Q/328L, 143A/145E/169S, 328L, 143A/145E/262S, 145E/262S/312I/328L, 139L/156V/157G, 139C/145E/312I, 312I, 139C, 139C/312I, 139C/156V, 139C/143A/145E/243L, 145E/157G, 145E/346T, 145E/262A/312I/328L/345R/346T, 145E/262A, 312I/342G, 143A/243L, 139C/345R, 342G, 143A/145E/262S/342G, 139L/143A/169S, 139C/143A/145E/312I, 169S, 139L/145E/262A/312I/328L/342G/345R/346T, 139C/328L, 139L/243L, 139L/143A/328L, 139L, 139C/143A/243L, 139C/145E, 145E/312I, 145E/169S, 139C/143A/157G/312I, 84M/139C/143A, 145E/269Q, 143A/145E/157G/269Q/312I/328L, 143A/145E/269Q, 157G, or 139L/143A/312I, wherein the amino acid positions are relative to the reference sequence corresponding to residues 135-413 of SEQ ID NO: 948, or relative to the reference sequence corresponding to SEQ ID NO: 948.

In some embodiments, the amino acid sequence of the engineered protease polypeptide comprises at least a substitution or substitution set S411V, S411R, A402G, A285S, I245V, N266H, P355A, M258W, A222G, Q140L, S268T, S411L, 1225V, 1245L, V283M, A406W, G410C, A406C, H143A/N145E/Q243L/S312I, N139L/H143A/N145E/S157G/S312I, N139C/S157G/T345R, N139C/M269Q, T156V/S157G/S342G/S346T, N139L/H143A, M269Q, N139L/Q243L/V328L, M269Q/V328L, H143A/N145E/T169S, V328L, H143A/N145E/G262S, N145E/G262S/S312I/V328L, N139L/T156V/S157G, N139C/N145E/S312I, S312I, N139C, N139C/S312I, N139C/T156V, N139C/H143A/N145E/Q243L, N145E/S157G, N145E/S346T, N145E/G262A/S312I/V328L/T345R/S346T, N145E/G262A, S312I/S342G, H143A/Q243L, N139C/T345R, S342G, H143A/N145E/G262S/S342G, N139L/H143A/T169S, N139C/H143A/N145E/S312I, T169S, N139L/N145E/G262A/S312I/V328L/S342G/T345R/S346T, N139C/V328L, N139L/Q243L, N139L/H143A/V328L, N139L, N139C/H143A/Q243L, N139C/N145E, N145E/S312I, N145E/T169S, N139C/H143A/S157G/S312I, V84M/N139C/H143A, N145E/M269Q, H143A/N145E/S157G/M269Q/S312I/V328L, H143A/N145E/M269Q, S157G, or N139L/H143A/S312I, wherein the amino acid positions are relative to the reference sequence corresponding to residues 135-413 of SEQ ID NO: 948, or relative to the reference sequence corresponding to SEQ ID NO: 948.

In some embodiments, the amino acid sequence of the engineered protease polypeptide comprises at least a substitution or substitution set, or amino acid residue(s) 256L, 411T, 273F, 409E, 268G, 172Q, 409R, 401L, 281C, 410W, 253V, 406M, 273T, 406R, 256M, 410I, 273M, 273L, 143A/145E/243L/328L, 145E, 139C/143A/145E/328L/342G/345R, 139L/145E, 143A/328L/342G/345R, 145E/342G/345R, 143A, 139C/143A, 139C/145E/328L/342G/345R, 143A/145E/169S/312I/328L/345R/346T, 143A/243L/328L/342G/345R/346T, 139C/143A/157G/169S/328L/346T, 143A/145E/156V/312I/328L, 139C/145E/157G/312I/328L, 143A/328L/342G/345R/346T, 139C/143A/328L, 143A/145E, 143A/145E/312I/342G/345R, or 143A/145E/328L, wherein the amino acid positions are relative to the reference sequence corresponding to residues 135-413 of SEQ ID NO: 948, or relative to the reference sequence corresponding to SEQ ID NO: 948.

In some embodiments, the amino acid sequence of the engineered protease polypeptide comprises at least a substitution or substitution set I256L, S411T, V273F, G409E, S268G, D172Q, G409R, H401L, T281C, G410W, A253V, A406M, V273T, A406R, 1256M, G410I, V273M, V273L, H143A/N145E/Q243L/V328L, N145E, N139C/H143A/N145E/V328L/S342G/T345R, N139L/N145E, H143A/V328L/S342G/T345R, N145E/S342G/T345R, H143A, N139C/H143A, N139C/N145E/V328L/S342G/T345R, H143A/N145E/T169S/S312I/V328L/T345R/S346T, H143A/Q243L/V328L/S342G/T345R/S346T, N139C/H143A/S157G/T169S/V328L/S346T, H143A/N145E/T156V/S312I/V328L, N139C/N145E/S157G/S312I/V328L, H143A/V328L/S342G/T345R/S346T, N139C/H143A/V328L, H143A/N145E, H143A/N145E/S312I/S342G/T345R, or H143A/N145E/V328L, wherein the amino acid positions are relative to the reference sequence corresponding to residues 135-413 of SEQ ID NO: 948, or relative to the reference sequence corresponding to SEQ ID NO: 948.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 1126, or to the reference sequence corresponding to SEQ ID NO: 1126, wherein the amino acid sequence comprises one or more substitutions relative to the reference sequence corresponding to residues 135-413 of SEQ ID NO: 1126, or relative to the reference sequence corresponding to SEQ ID NO: 1126.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 1156-1422, or to a reference sequence corresponding to an even-numbered SEQ ID NO. of SEQ ID NOs: 1156-1422, wherein the amino acid sequence comprises one or more substitutions relative to the reference sequence corresponding to residues 135-413 of SEQ ID NO: 1126, or relative to the reference sequence corresponding to SEQ ID NO: 1126.

In some embodiments, the amino acid sequence of the engineered protease polypeptide comprises at least a substitution or substitution set at amino acid position(s), or amino acid residue(s) 279, 250, 154, 214, 249, 275, 137, 161, 180, 174, 139, 254, 145, 278, 136, 154/413, 294, 237, 274, 264, 185, 277, 293, 233, 173, 312, 302, 238, 135, 221, 290, 263, 267, 239, 163, 292, 246, 243, 235, 156, 223, 278/413, 297, 194, 251, 253/411, 145/157/253/268/273/281/312/346/411, 139/346, 253, 346/411, 253/346, 312/346, 273/312, 253/281, 157/253/273/312/346/411, 139/157/253/268/273/281/312/346, 253/273/411, 139/253/268/273/281, 139/157/411, 157, 273, 139/253/268/273/281/312/411, 157/253/411, 139/145/253/346, 139/157/253/273/312, 139/157/268/273/312/346, 157/273/312/346, 139/411, 139/253/268/273/281/312/346/411, 157/273/346/411, 139/145/157/162/253/273/281/312, 139/253/273/281/312, 157/253/268/273/281/312, 139/253/268, 139/157/312, 253/273/281/346, 157/253/312/346/411, 157/273/312/346/411, 139/145/157/253/268/281/312, 139/273/312/346, 157/253/268/273/312/346, 139/268/346, 268/273/312/346, 139/157/253/268/273/312, 139/157/253, 139/253/281, 139/157/253/268/273, 253/312/411, or 139/268/273, wherein the amino acid positions are relative to the reference sequence corresponding to residues 135-413 of SEQ ID NO: 1126, or relative to the reference sequence corresponding to SEQ ID NO: 1126.

In some embodiments, the amino acid sequence of the engineered protease polypeptide comprises at least a substitution or substitution set, or amino acid residue(s) 279Y, 250T, 154C, 214Y, 249S, 275A, 137A, 161D, 180H, 174L, 139K, 254C, 145E, 278V, 136M, 154L/413D, 294V, 154R, 237A, 274G, 137N, 264P, 274T, 278N, 214A, 185G, 214V, 279T, 277G, 264I, 293A, 233L, 278Y, 173F, 274L, 312C, 279L, 302G, 238Q, 294W, 135A, 221E, 290S, 278S, 263Q, 263H, 278L, 263E, 154L, 139M, 137S, 233G, 139F, 267I, 221L, 173S, 302P, 221V, 239M, 290G, 163H, 292V, 246V, 214N, 243S, 233I, 235Q, 145D, 274V, 279M, 185S, 279K, 145W, 290E, 214P, 156V, 156C, 223S, 278V/413D, 250C, 267S, 297F, 221Q, 194D, 251T, 253V/411T, 145P/157R/253I/268G/273T/281V/312Q/346T/411T, 139C/346A, 253V, 346T/41 IT, 253V/346T, 139C/346T, 312Q/346T, 273T/312Q, 253I/281V, 157R/253V/273T/312Q/346T/411T, 139C/157K/253V/268G/273F/281V/312Q/346A, 253V/273T/411T, 139C/253I/268F/273T/281V, 139C/157R/411T, 157G, 273T, 139C/253V/268G/273F/281V/312Q/411T, 157G/253V/411T, 139C/145E/253V/346T, 139C/157K/253V/273T/312Q, 139C/157G/268G/273T/312Q/346T, 157G/273T/312Q/346T, 139C/411T, 139C/253V/268G/273F/281C/312I/346T/411T, 157K/273F/346T/411T, 139C/145E/157G/162I/253V/273F/281V/312Q, 139C/253V/273T/281C/312Q, 157G/253V/268F/273F/281V/312Q, 139C/253I/268F, 139C/157R/312Q, 253V/273T/281C/346T, 157K/253V/312I/346T/411T, 157R/273T/312Q/346T/411T, 139C/145E/157K/253V/268G/281C/312Q, 139C/273T/312Q/346T, 157K/253V/268F/273T/312I/346T, 139C/268G/346T, 157K, 268G/273F/312Q/346T, 139C/157K/253V/268F/273F/312Q, 157K/273T/312Q/346T, 139C/157G/253V, 139C, 139C/253V/281V, 139C/157G/253V/268G/273T, 253V/312Q/411T, 139C/268G/273T, or 253I, wherein the amino acid positions are relative to the reference sequence corresponding to residues 135-413 of SEQ ID NO: 1126, or relative to the reference sequence corresponding to SEQ ID NO: 1126.

In some embodiments, the amino acid sequence of the engineered protease polypeptide comprises at least a substitution or substitution set Q279Y, D250T, A154C, S214Y, A249S, T275A, H137A, S161D, N180H, N174L, N139K, D254C, N145E, A278V, V136M, A154L/G413D, S294V, A154R, S237A, Q274G, H137N, G264P, Q274T, A278N, S214A, N185G, S214V, Q279T, V277G, G264I, S293A, S233L, A278Y, H173F, Q274L, S312C, Q279L, S302G, L238Q, S294W, G135A, S221E, D290S, A278S, G263Q, G263H, A278L, G263E, A154L, N139M, H137S, S233G, N139F, Q267I, S221L, H173S, S302P, S221V, Y239M, D290G, K163H, A292V, L246V, S214N, Q243S, S233I, S235Q, N145D, Q274V, Q279M, N185S, Q279K, N145W, D290E, S214P, T156V, T156C, T223S, A278V/G413D, D250C, Q267S, Y297F, S221Q, N194D, 1251T, A253V/S411T, N145P/S157R/A253I/S268G/V273T/T281V/S312Q/S346T/S411T, N139C/S346A, A253V, S346T/S411T, A253V/S346T, N139C/S346T, S312Q/S346T, V273T/S312Q, A253I/T281V, S157R/A253V/V273T/S312Q/S346T/S411T, N139C/S157K/A253V/S268G/V273F/T281V/S312Q/S346A, A253V/V273T/S411T, N139C/A253I/S268F/V273T/T281V, N139C/S157R/S411T, S157G, V273T, N139C/A253V/S268G/V273F/T281V/S312Q/S411T, S157G/A253V/S411T, N139C/N145E/A253V/S346T, N139C/S157K/A253V/V273T/S312Q, N139C/S157G/S268G/V273T/S312Q/S346T, S157G/V273T/S312Q/S346T, N139C/S411T, N139C/A253V/S268G/V273F/T281C/S312I/S346T/S411T, S157K/V273F/S346T/S411T, N139C/N145E/S157G/V162I/A253V/V273F/T281V/S312Q, N139C/A253V/V273T/T281C/S312Q, S157G/A253V/S268F/V273F/T281V/S312Q, N139C/A253I/S268F, N139C/S157R/S312Q, A253V/V273T/T281C/S346T, S157K/A253V/S3I2I/S346T/S411T, S157R/V273T/S312Q/S346T/S411T, N139C/N145E/S157K/A253V/S268G/T281C/S312Q, N139C/V273T/S312Q/S346T, S157K/A253V/S268F/V273T/S312I/S346T, N139C/S268G/S346T, S157K, S268G/V273F/S312Q/S346T, N139C/S157K/A253V/S268F/V273F/S312Q, S157K/V273T/S312Q/S346T, N139C/S157G/A253V, N139C, N139C/A253V/T281V, N139C/S157G/A253V/S268G/V273T, A253V/S312Q/S411T, N139C/S268G/V273T, or A253I, wherein the amino acid positions are relative to the reference sequence corresponding to residues 135-413 of SEQ ID NO: 1126, or relative to the reference sequence corresponding to SEQ ID NO: 1126.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 1368, or to the reference sequence corresponding to SEQ ID NO: 1368, wherein the amino acid sequence comprises one or more substitutions relative to the reference sequence corresponding to residues 135-413 of SEQ ID NO: 1368, or relative to the reference sequence corresponding to SEQ ID NO: 1368.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 1424-1608, or to a reference sequence corresponding to an even-numbered SEQ ID NO. of SEQ ID NOs: 1424-1608, wherein the amino acid sequence comprises one or more substitutions relative to the reference sequence corresponding to residues 135-413 of SEQ ID NO: 1368, or relative to the reference sequence corresponding to SEQ ID NO: 1368.

In some embodiments, the amino acid sequence of the engineered protease polypeptide comprises at least a substitution or substitution set at amino acid position(s) 31, 318, 296, 252, 303, 253, 413, 386, 312, 235, 412, 342, 302, 371, 405, 389, 391, 358, 139/273/311/328/372, 311, 372, 139/143/157/160/268/273/311/315, 143, 139/143, 139/160/312/372, 143/273/328, 346, 135/139/160/268/312/342/346, 139/141/273, 135/141/143/268/273/312/372, 139/141/143/311, 139/157/268/328/346/372, 53/139/141/143/273/372, 139, 139/141/143/273/312, 137/139/221/233/413, 233, 221/279, 137/139/233/279, 221, 139/214, 137/139/156, 139/214/221, 214/233, 137/139/221/233/279, 137/139/221, 137/139, 137/139/279, 137, 137/139/214/279, 266, 139/221, 137/221, 137/156/214/312, 137/221/233, 137/139/156/221, 137/139/214, 279, 137/139/233, 137/139/214/233, 221/413, 137/214/233, 137/156, 137/139/221/233, 214/221, 137/221/413, 214, 137/233, 137/413, 137/221/279, 137/139/156/214/233/413, or 137/139/156/214, wherein the amino acid positions are relative to the reference sequence corresponding to residues 135-413 of SEQ ID NO: 1368, or relative to the reference sequence corresponding to SEQ ID NO: 1368.

In some embodiments, the amino acid sequence of the engineered protease polypeptide comprises at least a substitution or substitution set, or amino acid residue(s) 31G, 318R, 296R, 296M, 252P, 303A, 253C, 413C, 386P, 413A, 312R, 235R, 412P, 342A, 413S, 302P, 371L, 405L, 312A, 389P, 318P, 391S, 412T, 358S, 389C, 235V, 391L, 139N/273V/311T/328V/372S, 311T, 312S, 372S, 139N/143H/157S/160S/268S/273V/311T/315T, 143H, 139N/143H, 139N/160S/312S/372S, 143H/273V/328V, 346S, 135A/139N/160S/268S/312S/342S/346S, 139N/141N/273V, 135A/141N/143H/268S/273V/312S/372S, 139N/141N/143H/311T, 139N/157S/268S/328V/346S/372S, 53A/139N/141N/143H/273V/372S, 139N, 139N/141N/143H/273V/312S, 139L, 137A/139N/221Q/233L/413D, 233L, 221Q/279K, 137N/139N/233L/279M, 221Q, 139L/214V, 137N/139N/156V, 139L/214V/221Q, 214V/233L, 137A/139L/221Q/233L/279K, 137A/139L/221Q, 137N/139L, 137N/139L/279K, 137A/139N, 137N, 137N/139L/221Q, 137N/139L/214P/279M, 266Y, 139L/221Q, 137N/221Q, 137N/139N, 137N/156V/214V/312C, 137N/221Q/233L, 137N/139N/156V/221Q, 137A/139L/214V, 413D, 279K, 279M, 137A/139L/233L, 137N/139N/214V/233I, 221Q/413D, 137N/214V/233L, 137A/139L/214P, 137N/139N/221Q/233L/279M, 137N/156V, 137N/139L/221Q/233I, 214V/221Q, 137N/221Q/413D, 137A/139L/221Q/233L/279M, 214V, 137A/139L, 137N/233L, 137A, 137N/413D, 137N/221Q/279K, 137N/139N/156V/214V/233L/413D, or 137N/139L/156V/214V, wherein the amino acid positions are relative to the reference sequence corresponding to residues 135-413 of SEQ ID NO: 1368, or relative to the reference sequence corresponding to SEQ ID NO: 1368.

In some embodiments, the amino acid sequence of the engineered protease polypeptide comprises at least a substitution or substitution set D31G, S318R, S296R, S296M, D252P, S303A, A253C, G413C, E386P, G413A, Q312R, S235R, G412P, G342A, G413S, S302P, 1371L, V405L, Q312A, S389P, S318P, T391S, G412T, A358S, S389C, S235V, T391L, C139N/T273V/D311T/L328V/V372S, D311T, Q312S, V372S, C139N/A143H/G157S/L160S/G268S/T273V/D311T/V315T, A143H, C139N/A143H, C139N/L160S/Q312S/V372S, A143H/T273V/L328V, T346S, G135A/C139N/L160S/G268S/Q312S/G342S/T346S, C139N/V141N/T273V, G135A/V141N/A143H/G268S/T273V/Q312S/V372S, C139N/V141N/A143H/D311T, C139N/G157S/G268S/L328V/T346S/V372S, T53A/C139N/V141N/A143H/T273V/V372S, C139N, C139N/V141N/A143H/T273V/Q312S, C139L, H137A/C139N/S221Q/S233L/G413D, S233L, S221Q/Q279K, H137N/C139N/S233L/Q279M, S221Q, C139L/S214V, H137N/C139N/T156V, C139L/S214V/S221Q, S214V/S233L, H137A/C139L/S221Q/S233L/Q279K, H137A/C139L/S221Q, H137N/C139L, H137N/C139L/Q279K, H137A/C139N, H137N, H137N/C139L/S221Q, H137N/C139L/S214P/Q279M, N266Y, C139L/S221Q, H137N/S221Q, H137N/C139N, H137N/T156V/S214V/Q312C, H137N/S221Q/S233L, H137N/C139N/T156V/S221Q, H137A/C139L/S214V, G413D, Q279K, Q279M, H137A/C139L/S233L, H137N/C139N/S214V/S233I, S221Q/G413D, H137N/S214V/S233L, H137A/C139L/S214P, H137N/C139N/S221Q/S233L/Q279M, H137N/T156V, H137N/C139L/S221Q/S233I, S214V/S221Q, H137N/S221Q/G413D, H137A/C139L/S221Q/S233L/Q279M, S214V, H137A/C139L, H137N/S233L, H137A, H137N/G413D, H137N/S221Q/Q279K, H137N/C139N/T156V/S214V/S233L/G413D, or H137N/C139L/T156V/S214V, wherein the amino acid positions are relative to the reference sequence corresponding to residues 135-413 of SEQ ID NO: 1368, or relative to the reference sequence corresponding to SEQ ID NO: 1368.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 1548, or to the reference sequence corresponding to SEQ ID NO: 1548, wherein the amino acid sequence comprises one or more substitutions relative to the reference sequence corresponding to residues 135-413 of SEQ ID NO: 1548, or relative to the reference sequence corresponding to SEQ ID NO: 1548.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 1610-1710, or to a reference sequence corresponding to an even-numbered SEQ ID NO. of SEQ ID NOs: 1610-1710, wherein the amino acid sequence comprises one or more substitutions relative to the reference sequence corresponding to residues 135-413 of SEQ ID NO: 1548, or relative to the reference sequence corresponding to SEQ ID NO: 1548.

In some embodiments, the amino acid sequence of the engineered protease polypeptide comprises at least a substitution or substitution set at amino acid position(s) 145/273/372, 145/256/273, 221/243/273/328/372, 372, 145/221/279/372/406, 273/328, 145/169/273/346/406, 256/273, 145/221/273/328/346/406, 243/273, 145/214/256/273/279/328/372, 169/221/328/372/406, 372/406, 169/328/372/406, 221/372, 145/221/273/328/372, 214/243/273/328, 145/221, 214/256/273/346/372, 221/406, 169/372, 145/214/221/273, 145/221/346/372, 243/273/328/372/406, 145/169/273/328/346, 328, 169/273/372, 145/221/328, 169/214/273, 221/273/328, 221, 145/328, 214/346, 312, 212, 279, 212/312, 145, 179/346, 214, 346, 315/372, 375, 264, 179, 185, 220/372, or 324, wherein the amino acid positions are relative to the reference sequence corresponding to residues 135-413 of SEQ ID NO: 1548, or relative to the reference sequence corresponding to SEQ ID NO: 1548.

In some embodiments, the amino acid sequence of the engineered protease polypeptide comprises at least a substitution or substitution set, or amino acid residue(s) 145E/273L/372S, 145E/256M/273L, 221Q/243L/273L/328V/372S, 372S, 145E/221Q/279L/372S/406R, 273L/328V, 145E/169S/273L/346S/406R, 256M/273L, 145E/221Q/273L/328V/346S/406R, 243L/273L, 145E/214V/256M/273L/279L/328V/372S, 169S/221Q/328V/372S/406R, 372S/406R, 169S/328V/372S/406R, 221Q/372S, 145E/221Q/273L/328V/372S, 214V/243L/273L/328V, 145E/221Q, 214V/256M/273L/346S/372S, 221Q/406R, 169S/372S, 145E/214V/221Q/273L, 145E/221Q/346S/372S, 243L/273L/328V/372S/406R, 145E/169S/273L/328V/346S, 328V, 169S/273L/372S, 145E/221Q/328V, 169S/214V/273L, 221Q/273L/328V, 221Q, 145E/328V, 214N/346S, 312R, 212S, 279K, 212S/312R, 145E, 179S/346S, 214N, 346S, 315R/372Y, 372Y, 375A, 264F, 179S, 185T, 220R/372Y, 324D, 145E/221Q/273L/328V/372S, or 145E/221Q/273L/328V/372S, wherein the amino acid positions are relative to the reference sequence corresponding to residues 135-413 of SEQ ID NO: 1548, or relative to the reference sequence corresponding to SEQ ID NO: 1548.

In some embodiments, the amino acid sequence of the engineered protease polypeptide comprises at least a substitution or substitution set N145E/T273L/V372S, N145E/I256M/T273L, S221Q/Q243L/T273L/L328V/V372S, V372S, N145E/S221Q/M279L/V372S/A406R, T273L/L328V, N145E/T169S/T273L/T346S/A406R, I256M/T273L, N145E/S221Q/T273L/L328V/T346S/A406R, Q243L/T273L, N145E/P214V/I256M/T273L/M279L/L328V/V372S, T169S/S221Q/L328V/V372S/A406R, V372S/A406R, T169S/L328V/V372S/A406R, S221Q/V372S, N145E/S221Q/T273L/L328V/V372S, P214V/Q243L/T273L/L328V, N145E/S221Q, P214V/I256M/T273L/T346S/V372S, S221Q/A406R, T169S/V372S, N145E/P214V/S221Q/T273L, N145E/S221Q/T346S/V372S, Q243L/T273L/L328V/V372S/A406R, N145E/T169S/T273L/L328V/T346S, L328V, T169S/T273L/V372S, N145E/S221Q/L328V, T169S/P214V/T273L, S221Q/T273L/L328V, S221Q, N145E/L328V, P214N/T346S, Q312R, Y212S, M279K, Y212S/Q312R, N145E, A179S/T346S, P214N, T346S, V315R/V372Y, V372Y, Q375A, G264F, A179S, N185T, Q220R/V372Y, S324D, N145E/S221Q/T273L/L328V/V372S, or N145E/S221Q/T273L/L328V/V372S, wherein the amino acid positions are relative to the reference sequence corresponding to residues 135-413 of SEQ ID NO: 1548, or relative to the reference sequence corresponding to SEQ ID NO: 1548.

In some embodiments, the amino acid sequence of the engineered protease polypeptide comprises at least a substitution at an amino acid position set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the amino acid sequence of the engineered protease polypeptide comprises at least a substitution set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the amino acid sequence of the engineered protease polypeptide comprises at least a substitution or substitution set at amino acid position(s) set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the amino acid sequence of the engineered protease polypeptide comprises at least a substitution or substitution set of a reference engineered protease polypeptide set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence comprising at least a substitution or substitution set of an engineered protease polypeptide variant set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the amino acid sequence of the engineered protease polypeptide comprises amino acid residues 135-413, or comprises amino acid residues 128-413, wherein the engineered protease polypeptide is proteolytically active or is an active protease.

In some embodiments, the engineered protease polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to an amino acid sequence comprising residues 135-413 of an amino acid sequence of a protease variant set forth in Tables 3.1, 3.2, 4.1, 4.2, 5.1, 5.2, 6.1, 7.1, 8.1, and 9.1, or to an amino acid sequence comprising an amino acid sequence of a variant set forth in Tables 3.1, 3.2, 4.1, 4.2, 5.1, 5.2, 6.1, 7.1, 8.1, and 9.1.

In some embodiments, the engineered protease polypeptide comprises an amino acid sequence comprising residues 135-413, or residues 128-413 of SEQ ID NO: 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, 1410, 1412, 1414, 1416, 1418, 1420, 1422, 1424. 1426, 1428, 1430, 1432, 1434, 1436, 1438, 1440, 1442, 1444, 1446, 1448, 1450, 1452, 1454, 1456, 1458, 1460, 1462, 1464, 1468, 1470, 1472, 1474, 1476, 1478, 1480, 1482, 1484, 1486, 1488, 1490, 1492, 1494, 1496, 1498, 1500, 1502, 1504, 1506, 1508, 1510, 1512, 1514, 1516, 1518, 1520, 1522, 1524, 1526, 1528, 1530, 1532, 1534, 1536, 1538, 1540, 1542, 1544, 1546, 1548, 1550, 1552, 1554, 1556, 1558, 1560, 1562, 1564, 1566, 1568, 1570, 1572, 1574, 1576, 1578, 1580, 1582, 1584, 1586, 1588, 1590, 1592, 1594, 1596, 1598, 1600, 1602, 1604, 1606, 1608, 1610, 1612, 1614, 1616, 1618, 1620, 1622, 1624, 1626, 1628, 1630, 1632, 1634, 1636, 1638, 1640, 1642, 1644, 1646, 1648, 1650, 1652, 1654, 1656, 1658, 1660, 1662, 1664, 1666, 1668, 1670, 1672, 1674, 1676, 1678, 1680, 1682, 1684, 1686, 1688, 1690, 1692, 1694, 1696, 1698, 1700, 1702, 1704, 1706, 1708, 1710, 1712, 1714, 1716, 1718, 1720, 1722, 1724, 1726, 1728, 1730, 1732, 1734, 1736, 1738, 1740, 1742, 1744, 1746, 1748, 1750, 1752, 1754, 1756, 1758, 1760, 1762, 1764, 1766, 1768, 1770, 1772, 1774, 1776, 1778, 1780, 1782, 1784, 1786, 1788, 1790, 1792, 1794, 1796, 1798, 1800, 1802, 1804, 1806, 1808, 1810, 1812, 1814, 1816, 1818, 1820, 1822, 1824, 1826, 1828, 1830, 1832, 1834, 1836, 1838, 1840, 1842, 1844, 1846, 1848, 1850, 1852, 1854, 1856, 1858, 1860, 1862, 1864, 1866, 1868, 1870, 1872, 1874, 1876, 1878, 1880, 1882, 1884, 1886, 1888, 1890, 1892, 1894, 1896, 1898, 1900, 1902, 1904, 1906, 1908, 1910, 1912, 1914, 1916, 1918, 1920, 1922, 1924, 1926, 1928, 1930, 1932, 1934, 1936, 1938, 1940, 1942, 1944, 1946, 1948, 1950, 1952, 1954, 1956, 1958, 1960, 1962, 1964, 1966, 1968, 1970, 1972, 1974, 1976, 1978, 1980, 1982, 1984, 1986, 1988, 1990, 1992, 1994, 1996, 1998, 2000, 2002, 2004, 2006, 2008, 2010, 2012, 2014, 2016, 2018, 2020, 2022, 2024, 2026, 2028, 2030, 2032, 2034, 2036, 2038, 2040, 2042, 2044, 2046, 2048, 2050, 2052, 2054, 2056, 2058, 2060, 2062, 2064, 2066, 2068, 2070, 2072, 2074, 2076, 2078, 2080, 2082, 2084, 2086, 2088, 2090, 2092, 2094, 2096, 2098, 2100, 2102, 2104, 2106, 2108, 2110, 2112, 2114, 2116, 2118, 2120, 2122, 2124, 2126, 2128, 2130, 2132, 2134, 2136, 2138, 2140, 2142, 2144, 2146, 2148, 2150, 2152, 2154, 2156, 2158, 2160, 2162, 2164, 2166, 2168, 2170, 2172, 2174, 2176, 2178, 2180, 2182, 2184, 2186, 2188, 2190, 2192, 2194, 2196, 2198, 2200, 2202, 2204, 2206, 2208, 2210, 2212, 2214, 2216, 2218, 2220, 2222, 2224, 2226, 2228, 2230, 2232, 2234, 2236, 2238, 2240, or 2242. In some embodiments, the amino acid sequence of the engineered protease polypeptide optionally has 1, 2, 3, 4, 5, 6, 7, 8, 9, or up to 10 insertions, substitutions, and/or deletions. In some embodiments, the amino acid sequence optionally has 1, 2, 3, 4, 5, 6, 7, 8, 9, or up to 10 substitutions. In some of embodiments, the amino acid sequence of the engineered protease polypeptide optionally has 1, 2, 3, 4, up to 5 insertions, substitutions, and/or deletions. In some embodiments, the amino acid sequence optionally has 1, 2, 3, 4, up to 5 substitutions. In some embodiments, the substitutions comprise non-conservative and/or conservative substitutions. In some embodiments, the substitutions comprise conservative substitutions.

In some embodiments, the engineered protease polypeptide comprises an amino acid sequence comprising SEQ ID NO: 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, 1410, 1412, 1414, 1416, 1418, 1420, 1422, 1424. 1426, 1428, 1430, 1432, 1434, 1436, 1438, 1440, 1442, 1444, 1446, 1448, 1450, 1452, 1454, 1456, 1458, 1460, 1462, 1464, 1468, 1470, 1472, 1474, 1476, 1478, 1480, 1482, 1484, 1486, 1488, 1490, 1492, 1494, 1496, 1498, 1500, 1502, 1504, 1506, 1508, 1510, 1512, 1514, 1516, 1518, 1520, 1522, 1524, 1526, 1528, 1530, 1532, 1534, 1536, 1538, 1540, 1542, 1544, 1546, 1548, 1550, 1552, 1554, 1556, 1558, 1560, 1562, 1564, 1566, 1568, 1570, 1572, 1574, 1576, 1578, 1580, 1582, 1584, 1586, 1588, 1590, 1592, 1594, 1596, 1598, 1600, 1602, 1604, 1606, 1608, 1610, 1612, 1614, 1616, 1618, 1620, 1622, 1624, 1626, 1628, 1630, 1632, 1634, 1636, 1638, 1640, 1642, 1644, 1646, 1648, 1650, 1652, 1654, 1656, 1658, 1660, 1662, 1664, 1666, 1668, 1670, 1672, 1674, 1676, 1678, 1680, 1682, 1684, 1686, 1688, 1690, 1692, 1694, 1696, 1698, 1700, 1702, 1704, 1706, 1708, 1710, 1712, 1714, 1716, 1718, 1720, 1722, 1724, 1726, 1728, 1730, 1732, 1734, 1736, 1738, 1740, 1742, 1744, 1746, 1748, 1750, 1752, 1754, 1756, 1758, 1760, 1762, 1764, 1766, 1768, 1770, 1772, 1774, 1776, 1778, 1780, 1782, 1784, 1786, 1788, 1790, 1792, 1794, 1796, 1798, 1800, 1802, 1804, 1806, 1808, 1810, 1812, 1814, 1816, 1818, 1820, 1822, 1824, 1826, 1828, 1830, 1832, 1834, 1836, 1838, 1840, 1842, 1844, 1846, 1848, 1850, 1852, 1854, 1856, 1858, 1860, 1862, 1864, 1866, 1868, 1870, 1872, 1874, 1876, 1878, 1880, 1882, 1884, 1886, 1888, 1890, 1892, 1894, 1896, 1898, 1900, 1902, 1904, 1906, 1908, 1910, 1912, 1914, 1916, 1918, 1920, 1922, 1924, 1926, 1928, 1930, 1932, 1934, 1936, 1938, 1940, 1942, 1944, 1946, 1948, 1950, 1952, 1954, 1956, 1958, 1960, 1962, 1964, 1966, 1968, 1970, 1972, 1974, 1976, 1978, 1980, 1982, 1984, 1986, 1988, 1990, 1992, 1994, 1996, 1998, 2000, 2002, 2004, 2006, 2008, 2010, 2012, 2014, 2016, 2018, 2020, 2022, 2024, 2026, 2028, 2030, 2032, 2034, 2036, 2038, 2040, 2042, 2044, 2046, 2048, 2050, 2052, 2054, 2056, 2058, 2060, 2062, 2064, 2066, 2068, 2070, 2072, 2074, 2076, 2078, 2080, 2082, 2084, 2086, 2088, 2090, 2092, 2094, 2096, 2098, 2100, 2102, 2104, 2106, 2108, 2110, 2112, 2114, 2116, 2118, 2120, 2122, 2124, 2126, 2128, 2130, 2132, 2134, 2136, 2138, 2140, 2142, 2144, 2146, 2148, 2150, 2152, 2154, 2156, 2158, 2160, 2162, 2164, 2166, 2168, 2170, 2172, 2174, 2176, 2178, 2180, 2182, 2184, 2186, 2188, 2190, 2192, 2194, 2196, 2198, 2200, 2202, 2204, 2206, 2208, 2210, 2212, 2214, 2216, 2218, 2220, 2222, 2224, 2226, 2228, 2230, 2232, 2234, 2236, 2238, 2240, or 2242. In some embodiments, the amino acid sequence of the engineered protease polypeptide optionally has 1, 2, 3, 4, 5, 6, 7, 8, 9, or up to 10 insertions, substitutions, and/or deletions. In some embodiments, the amino acid sequence optionally has 1, 2, 3, 4, 5, 6, 7, 8, 9, or up to 10 substitutions. In some of embodiments, the amino acid sequence of the engineered protease polypeptide optionally has 1, 2, 3, 4, up to 5 insertions, substitutions, and/or deletions. In some embodiments, the amino acid sequence optionally has 1, 2, 3, 4, up to 5 substitutions. In some embodiments, the substitutions comprise non-conservative and/or conservative substitutions. In some embodiments, the substitutions comprise conservative substitutions.

In some embodiments, the engineered protease polypeptide comprises an amino acid sequence comprising residues 135-413 of SEQ ID NO: 628, 948, 1126, 1368, 1547, 1640, or 1710, or an amino acid sequence comprising SEQ ID NO: 628, 948, 1126, 1368, 1547, 1640, or 1710. In some embodiments, the amino acid sequence of the engineered protease polypeptide optionally has 1, 2, 3, 4, 5, 6, 7, 8, 9, or up to 10 insertions, substitutions, and/or deletions. In some embodiments, the amino acid sequence optionally has 1, 2, 3, 4, 5, 6, 7, 8, 9, or up to 10 substitutions. In some of embodiments, the amino acid sequence of the engineered protease polypeptide optionally has 1, 2, 3, 4, up to 5 insertions, substitutions, and/or deletions. In some embodiments, the amino acid sequence optionally has 1, 2, 3, 4, up to 5 substitutions. In some embodiments, the substitutions comprise non-conservative and/or conservative substitutions. In some embodiments, the substitutions comprise conservative substitutions.

In some embodiments, the engineered protease polypeptide described herein, particularly the pro-polypeptide form, is capable of converting or forming a proteolytically active polypeptide or an active protease. In some embodiments, the formation of a proteolytically active polypeptide or an active protease is by auto-proteolysis. In some embodiments, the formation of a proteolytically active polypeptide or an active protease is by proteolysis by another protease, including any of the proteolytically active polypeptide or active protease of the engineered protease polypeptide described herein.

In some embodiments, the engineered protease polypeptide comprises a proteolytically active polypeptide or is an active protease. In some embodiments, the proteolytically active polypeptide or active protease comprises amino acid residues 135-413, or comprises amino acid residues 128-413 of any of the engineered protease polypeptide described herein.

In some embodiments, the proteolytically active polypeptide or active protease of the engineered protease polypeptide has an improved property as compared to a reference protease polypeptide.

In some embodiments, the proteolytically active polypeptide or active protease of the engineered protease polypeptide has increased protease activity as compared to a reference protease. In some embodiments, the proteolytically active polypeptide or active protease of the engineered protease polypeptide has at least 1.1, 1.2, 1.3, 1.4, 1.5, 2, 2.5, 3, 3.5, 4, 4.5, or 5 or greater fold increase in protease activity as compared to the reference protease. Exemplary increases in protease activity compared to the reference protease are provided in the Examples.

In some embodiments, the proteolytically active polypeptide or active protease of the engineered protease polypeptide has increased resistance to a gastric protease as compared to a reference protease. In some embodiments, the proteolytically active polypeptide or active protease of the engineered protease polypeptide has increased resistance to pepsin as compared to the reference protease. In some embodiments, the increased resistance to the gastric protease is at acidic pH conditions.

In some embodiments, the proteolytically active polypeptide or active protease of the engineered protease polypeptide has increased stability and/or activity at acidic pH or neutral pH as compared to a reference protease. In some embodiments, the proteolytically active polypeptide or active protease of the engineered protease polypeptide has at least 1.1, 1.2, 1.3, 1.4, 1.5, 2, 2.5, 3, 3.5, 4, 4.5, or 5 or greater fold increase in protease activity at acidic pH or neutral as compared to the reference protease. In some embodiments, the acidic pH is from about 2.8 to 4.5.

In some embodiments, the proteolytically active polypeptide or active protease of the engineered protease polypeptide has increased thermostability as compared to a reference protease. In some embodiments, the proteolytically active polypeptide or active protease of the engineered protease polypeptide has increased thermostability at temperature of about 64° C. or 71° C. as compared to the reference protease.

In some embodiments, the proteolytically active polypeptide or active protease of the engineered protease polypeptide is characterized by an improved property selected from: i) increased protease activity, ii) increased resistance to pepsin, iii) increased stability and/or activity at acidic pH, iv) increased stability and/or activity at neutral pH, or v) increased thermostability, or any combination of i), ii), iii), iv), and v) as compared to a reference protease.

In some embodiments, the reference protease has an amino acid sequence corresponding to residues 135-413 of SEQ ID NO: 4, 628, 948, 1126, 1368, or 1548; residues 128-413 of SEQ ID NO: 4, 628, 948, 1126, 1368, or 1548; or a mature active protease of SEQ ID NO: 4, 628, 948, 1126, 1368, or 1548. In some embodiments, the reference protease has an amino acid sequence corresponding to residues 135-413 or residues 128-413 of SEQ ID NO: 4 or 628, or a mature active protease of SEQ ID NO: 4 or 628. In some embodiments, the reference protease is a proteolytically active polypeptide of SEQ ID NO: 2.

In some embodiments, the pro-polypeptide or proteolytically active polypeptide of the engineered protease polypeptide described herein includes or further comprises at the carboxy terminal region a Big-1 domain. In some embodiments, the Big-1 domain is that of SEQ ID NO: 2. In some embodiments, the Big-1 domain comprises amino acid residues 426-522 of SEQ ID NO: 2.

In some embodiments, the engineered protease polypeptide comprises a deletion of the protease of SEQ ID NO: 2, where the deletion is of at least the carboxy terminal region of SEQ ID NO: 2, and wherein the deletion maintains the protease activity of the mature form of SEQ ID NO: 2. In some embodiments, the carboxy terminal region deleted comprises deletion of the Big-1 domain. In some embodiments, the carboxy terminal deletion is up to and including amino acid residue 426, or up to and including amino acid residue 414 of SEQ ID NO: 2. In some embodiments, the engineered protease polypeptide further comprises 1, 2, 3, 4, 5, 6, 7, 8, 9, or up to 10 amino acid deletions of the carboxy terminus at amino acid residue 413 of SEQ ID NO: 2, wherein the further amino acid deletion(s) maintain proteolytic activity of the mature form of SEQ ID NO: 2 having the further amino acid deletions. In some embodiments, the mature form of the protease polypeptide has an amino terminus at amino acid residue 128 or 135 of SEQ ID NO: 2.

In some embodiments, the engineered protease polypeptide further comprises a fusion polypeptide or a fusion protein. In some embodiments, the engineered protease polypeptide further comprises a heterologous fusion polypeptide or a fusion protein. In some embodiments, the engineered protease polypeptide described herein can be fused to a variety of polypeptide or protein sequences, such as, by way of example and not limitation, polypeptide tags that can be used for detection and/or purification. In some embodiments, the fusion polypeptide of the recombinant protease polypeptide comprises a glycine-histidine or histidine-tag (His-tag). In some embodiments, the fusion polypeptide of the engineered protease polypeptide comprises an epitope tag, such as c-myc, FLAG, V5, or hemagglutinin (HA). In some embodiments, the fusion polypeptide of the engineered protease polypeptide comprises a GST, SUMO, Strep, MBP, or GFP tag. In some embodiments, the fusion is to the amino (N-) terminus of the engineered protease polypeptide. In some embodiments, the fusion is to the carboxy (C-) terminus of the engineered protease polypeptide. In some embodiments, the fusion polypeptide is inserted following a signal sequence and before the engineered protease polypeptide to allow expression and secretion of the protease polypeptide comprising the fusion polypeptide (e.g., polypeptide tag) and engineered protease polypeptide.

In some embodiments, the engineered protease polypeptide further comprises a signal sequence or signal peptide. In some embodiments, the signal sequence or signal peptide is functional in the host cell used or to be used for expression of the engineered protease polypeptide. In some embodiments the signal sequence or signal peptide is fused to a pro-polypeptide form of the engineered protease polypeptide, e.g., for forming a pre-pro-polypeptide. In some embodiments, the signal sequence or signal peptide is fused to the polypeptide that includes the proteolytically active polypeptide or active protease of the engineered protease polypeptide. In some embodiments, the signal sequence or signal peptide can be a bacterial, fungal, or mammalian signal sequence or signal polypeptide. In some embodiments, the signal sequence or signal peptide can be a naturally occurring signal sequence or a synthetic signal sequence, including a hybrid signal sequence.

In some embodiments, the signal sequence or signal peptide is a bacterial signal sequence or signal peptide, or a signal sequence or signal peptide functional in bacterial cells. In some embodiments, the bacterial signal sequence or signal peptide is recognized by the general secretion (Sec) or twin-arginine translocation (Tat) pathway (see, e.g., Freudl, R., Microbial Cell Factories, 2018, 17(52):1-10). In some embodiments, the signal sequence or signal peptide can be a Sec signal sequence or signal peptide from genes encoding, among others, LamB, MalE, OmpA, OmpF, OmpN, OmpC, OmpX, PhoA, PhoE, GBP, TolC, TolB, or CirA. In some embodiments, the signal sequence or signal peptide can be a Tat signal sequence or signal peptide from genes encoding, among others, TorA, TorZ, AmiA, AmiC, FtsP, EfeB, YcbK, NrfC, WcaM, YahJ, MdoD, or FhuD.

In some embodiments, signal sequence or signal peptide is a fungal (e.g., yeast) signal sequence or signal peptide or a signal sequence, or a signal peptide functional in fungal cells. Exemplary fungal signal sequence or signal peptide includes, among others, those found in Pichia pastoris Ost1, Pichia pastoris Pst1, S. cerevisiae α-mating factor pre-pro sequence, S. cerevisiae invertase, Komagataella pastoris yeast α-factor, S. cerevisiae CYP, Pichia pastoris PH08, S. cerevisiae PEP4, S. cerevisiae SUC2, Pichia pastoris KAR2, Pichia pastoris DSE4, Pichia pastoris EXG1, or Pichia pastoris SCW10.

In some embodiments, the signal sequence or signal peptide is a mammalian or insect cell signal sequence or signal peptide, or a signal sequence or signal peptide functional in mammalian cells or insect cells. Exemplary mammalian or insect signal sequence or signal peptide includes those from, among others, human OSM, VSV-G, mouse Ig Kappa, mouse Ig heavy, BM40, Secrecon, human IgKVIII, CD33, tPA, human chymotrypsinogen, human trypsinogen-2, human IL-2, human serum albumin (HSA), influenza haemagglutinin, human insulin, silkworm Fibroin LC, and honeybee melittin signal peptide of gp64 or gp67.

In some embodiments, for any of the engineered protease polypeptide disclosed herein, the engineered protease polypeptide is purified or is a purified preparation or composition. In some embodiments, the purified preparation comprises the pro-polypeptide of the engineered protease polypeptide. In some embodiments, the purified preparation comprises the proteolytically active polypeptide or the active protease form of the engineered protease polypeptide.

In some embodiments, the present disclosure further provides a functional or biologically active fragment of an engineered protease polypeptide described herein. In some embodiments, functional or biologically active fragments have at least 80%, 81%, 82%, 83%, 84%, 85%, 86%, 87%, 88%, 89%, 90%, 91%, 92%, 93%, 94%, 95%, 96%, 97%, 98%, or 99% of the activity of the proteolytically active polypeptide or the active protease of the engineered protease polypeptide from which it was derived (i.e., the parent engineered protease polypeptide). In some embodiments, a functional or biologically active fragment comprises at least 80%, 81%, 82%, 83%, 84%, 85%, 86%, 87%, 88%, 89%, 90%, 91%, 92%, 93%, 94%, 95%, 96%, 97%, 98%, or 99% of the parent sequence of the engineered protease polypeptide.

In some embodiments the functional or biologically active fragment is truncated by less than 5, less than 10, less than 15, less than 10, less than 25, less than 30, less than 35, less than 40, less than 45, and less than 50 amino acids of the parent engineered protease polypeptide.

In some embodiments, the functional or biologically active fragment of the engineered protease polypeptide described herein includes at least a mutation or mutation set in the amino acid sequence of the parent engineered protease polypeptide variant described herein. Accordingly, in some embodiments, the functional or biologically active fragments of the engineered protease polypeptide displays the enhanced or improved property associated with the mutation or mutation set in the parent engineered protease polypeptide variant.

### Polynucleotides Encoding Engineered Protease Polypeptides, Expression Vectors, and Host Cells

In a further aspect, the present disclosure provides a recombinant polynucleotide encoding an engineered protease polypeptide described herein, expression vectors comprising the recombinant polynucleotide operably linked to one or more control sequences, and appropriate host cells comprising the expression vector for expression of the engineered protease polypeptide.

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode the engineered protease polypeptide. Thus, having knowledge of a particular amino acid sequence, those skilled in the art could make any number of different nucleic acids by simply modifying the sequence of one or more codons in a way which does not change the amino acid sequence of the encoded protein. In this regard, the present disclosure specifically contemplates each and every possible variation of polynucleotides that could be made encoding the engineered protease polypeptide described herein by selecting combinations based on the possible codon choices, and all such polynucleotide variations are to be considered specifically disclosed for any polypeptide described herein, including the engineered protease polypeptide set forth in Tables 3.1, 3.2, 4.1, 4.2, 5.1, 5.2, 6.1, 7.1, 8.1, and 9.1, and the accompanying Sequence Listing.

In some embodiments, the codons are preferably optimized for utilization by the chosen host cell for protein production. In some embodiments, the polynucleotide encoding the engineered protease polypeptide preferably uses preferred codons used in bacterial cells for expression in bacterial cells. In some embodiments, the polynucleotide encoding the engineered protease polypeptide preferably uses preferred codons used in fungal cells for expression in fungal cells. In some embodiments, the polynucleotide encoding the engineered protease polypeptide preferably uses preferred codons used in insect cells for expression insect cells. In some embodiments, the polynucleotide encoding the engineered protease polypeptide preferably uses preferred codons used in mammalian cells for expression in mammalian cells. In some embodiments, codon optimized polynucleotides encoding an engineered protease polypeptide described herein contain preferred codons at about 40%, 50%, 60%, 70%, 80%, 90%, or greater than 90% of the codon positions in the coding region.

As discussed above, it is to be understood that the present disclosure provides recombinant polynucleotides encoding each and every engineered protease polypeptide described herein, including a pre-pro-polypeptide, pro-polypeptide, or proteolytically active polypeptide thereof, or a biologically or functionally active fragment thereof.

In some embodiments, the recombinant polynucleotide comprises a polynucleotide sequence encoding an engineered protease polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 135-413 of SEQ ID NO: 4, 628, 948, 1126, 1368, or 1548, or to the reference sequence corresponding to SEQ ID NO: 4, 628, 948, 1126, 1368, or 1548, wherein the amino acid sequence comprises one or more substitutions relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 4 or 628, or to the reference sequence corresponding to SEQ ID NO: 4 or 628, wherein the amino acid sequence comprises one or more substitutions relative to the reference sequence corresponding to residues 135-413 of SEQ ID NO: 4 or 628, or relative to the reference sequence corresponding to SEQ ID NO: 4 or 628.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 948, 1126, 1368, or 1548, or to the reference sequence corresponding to SEQ ID NO: 948, 1126, 1368, or 1548, wherein the amino acid sequence comprises one or more substitutions relative to the reference sequence corresponding to residues 135-413 of SEQ ID NO: 4 or 628, or relative to the reference sequence corresponding to SEQ ID NO: 4 or 628.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of an even-numbered SEQ ID NO. of SEQ ID NOs: 6-1710, or to the reference sequence corresponding to an even-numbered SEQ ID NO. of SEQ ID NOs: 6-1710, wherein the amino acid sequence comprises one or more substitutions relative to the reference sequence corresponding to residues 135-413 of SEQ ID NO: 4 or 628, or relative to the reference sequence corresponding to SEQ ID NO: 4 or 628.

In some embodiments, the recombinant polynucleotide comprises a polynucleotide sequence encoding an engineered protease polypeptide comprising an amino acid sequence comprising at least a substitution at amino acid position 11, 31, 42, 45, 50, 53, 84, 99, 100, 126, 128, 129, 130, 131, 132, 134, 135, 136, 137, 138, 139, 140, 141, 143, 145, 151, 154, 156, 157, 159, 160, 161, 162, 163, 169, 172, 173, 174, 179, 180, 184, 185, 186, 187, 188, 190, 191, 192, 193, 194, 198, 199, 212, 214, 220, 221, 222, 223, 225, 231, 232, 233, 235, 237, 238, 239, 240, 242, 243, 245, 246, 249, 250, 251, 252, 253, 254, 256, 258, 262, 263, 264, 265, 266, 267, 268, 269, 271, 273, 274, 275, 277, 278, 279, 280, 281, 283, 285, 290, 292, 293, 294, 296, 297, 300, 302, 303, 311, 312, 313, 314, 315, 316, 318, 324, 328, 336, 339, 341, 342, 343, 345, 346, 355, 358, 360, 364, 367, 368, 369, 370, 371, 372, 373, 374, 375, 377, 381, 382, 384, 386, 389, 391, 392, 401, 402, 405, 406, 409, 410, 411, 412, or 413, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the recombinant polynucleotide comprises a polynucleotide sequence encoding an engineered protease polypeptide comprising an amino acid sequence comprising at least a substitution at amino acid position 135, 137, 139, 141, 143, 157, 160, 214, 268, 273, 279, 311, 312, 315, 328, 342, 345, 346, or 372, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the recombinant polynucleotide comprises a polynucleotide sequence encoding an engineered protease polypeptide comprising an amino acid sequence comprising at least a substitution set at amino acid positions 135/141/160/311/315/372, 143/328/342/345, 139/157/268/273/312/346, or 137/139/214/279, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the recombinant polynucleotide comprises a polynucleotide sequence encoding an engineered protease polypeptide comprising an amino acid sequence comprising at least a substitution or substitution set at amino acid position(s) 185, 134, 129, 135, 184, 132, 186, 193, 263, 370, 45/134, 199, 368, 161, 141, 267, 179, 264, 160, 138, 131, 372, 151, 274, 128, 339, 313, 374, 314, 191, 324, 315, 375, 136, 220, 194, 231, 277, 369, 251, 180, 163, 343, 264/279, 279, 232, 141/300, 367, 266, 188, 130, 318, 265, 341, 190, 145, 126/192, 11/220, 192, 370/392, 99/278, 265/311, 84/159/265/279/311/370, 311/316, 342/370, 265/311/370, 192/311/316, 141/154/192, 265/311/316/342, 279/311/316, 141/265/279/311/342, 141/192/311/316/370, 141/265/311, 198/279, 392, 342/370/392, 141/198/265, 265/392, 184/267, 342, 312, 100/251, 141/220, 311/316/370, 99, 278, 405, 311/342/370, 141/198, 311/342, 141/311, 279/311/377/392, 186/198/311/342/370/392, 141/392, 311/370/392, 141/311/392, 311/370, 311/316/392, 265/311/392, 141/192, 311, 141/265/311/392, 192/311/370/392, 198/265/311/316/370, 141/186/265/311, or 141/198/265/311/370, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the recombinant polynucleotide comprises a polynucleotide sequence encoding an engineered protease polypeptide comprising an amino acid sequence comprising at least a substitution or substitution set at amino acid position(s) 242, 157, 250, 373, 243, 336, 187, 240, 280, 271, 237, 386, 382, 328, 42, 391, 381, 275, 249, 239, 384, 139, 364, 346, 389, 254, 246, 345, 360, 303, 300, 269, 135/141/372, 311/315/372, 136/141/311, 141/188, 135/136, 135/141/315, 372, 135/141/160/267/372, 135/136/141/160/185/188/267/311/315, 160/185, 135/141/188/279/311, 135/136/141, 135/136/141/372, 135/141/160/185/267/279, 135/141/160/267, 141/188/311/372, 160/185/188/279/311, 136/141/279, 135/136/141/160/185/188, 141/372, 135/136/141/311, 185/311/315/372, 135/141/188, 136/185, 135/141, 135/136/141/279/315/372, 135/311/315, 141, 311/372, 188/311, 135/141/188/372, 141/160/279, 313/392, 342/392, 279/392, 128, 198/342, 313, 128/312, 50, 145/263, 313/342, 279/312, 312/392, 279/342, 128/342, 342, 263, 143, 262, 156, 169, 143/237, 136/160/185/267/311/372, 135/160/311/372, 135/141/311/315, 141/311/315, 136/141/160/185/188/311/315/372, 135/141/311/315/372, 135/141/160/185/311/315, 135/141/267/311/315/372, 135/136/141/160/311/315, 135/136/141/279, 135/141/267/279/311/315, 135/141/160, 135/141/160/311/315/372, 135/141/160/311/315, 135/136/141/188/311, 141/160/311, 135/141/160/279/311/315/372, 141/160/185/279/311/372, 135/136/141/160/315/372, 135/136/160/279/311/372, 128/279/312/342, 128/198/312/342, 263/342, 145/263/279/312/342/392, or 128/145/198/312/313/392, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the recombinant polynucleotide comprises a polynucleotide sequence encoding an engineered protease polypeptide comprising an amino acid sequence comprising at least a substitution at amino acid position 135, 137, 139, 141, 143, 145, 145, 157, 160, 214, 221, 268, 273, 279, 311, 312, 315, 315, 342, 345, 346, 402, 409, 410, 412, or 413, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the recombinant polynucleotide comprises a polynucleotide sequence encoding an engineered protease polypeptide comprising an amino acid sequence comprising at least a substitution at an amino acid position set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the recombinant polynucleotide comprises a polynucleotide sequence encoding an engineered protease polypeptide comprising an amino acid sequence comprising at least a substitution as set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the recombinant polynucleotide comprises a polynucleotide sequence encoding an engineered protease polypeptide comprising an amino acid sequence comprising at least a substitution or substitution set at amino acid position(s) set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the recombinant polynucleotide comprises a polynucleotide sequence encoding an engineered protease polypeptide comprising an amino acid sequence comprising at least a substitution or substitution set of an engineered protease polypeptide set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence having a substitution or substitution set as set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4 or 628, or relative to the reference sequence corresponding to SEQ ID NO: 4 or 628.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 948, 1126, 1368, or 1548, or to the reference sequence corresponding to SEQ ID NO: 948, 1126, 1368, or 1548.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of an even-numbered SEQ ID NO. of SEQ ID NOs: 6-2242, or to the reference sequence corresponding to an even-numbered SEQ ID NO. of SEQ ID NOs: 6-2242.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 948, 1126, 1368, or 1548, or to the reference sequence corresponding to SEQ ID NO: 948, 1126, 1368, or 1548, wherein the amino acid sequence comprises one or more substitutions relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of an even-numbered SEQ ID NO. of SEQ ID NOs: 6-2242, or to the reference sequence corresponding to an even-numbered SEQ ID NO. of SEQ ID NOs: 6-2242, wherein the amino acid sequence comprises one or more substitutions relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the recombinant polynucleotide comprises a polynucleotide sequence encoding an engineered protease polypeptide comprising at least a substitution at amino acid position 11, 31, 42, 45, 50, 53, 84, 99, 100, 126, 128, 129, 130, 131, 132, 134, 135, 136, 137, 138, 139, 140, 141, 143, 145, 151, 154, 156, 157, 159, 160, 161, 162, 163, 169, 172, 173, 174, 179, 180, 184, 185, 186, 187, 188, 190, 191, 192, 193, 194, 198, 199, 212, 214, 220, 221, 222, 223, 225, 231, 232, 233, 235, 237, 238, 239, 240, 242, 243, 245, 246, 249, 250, 251, 252, 253, 254, 256, 258, 262, 263, 264, 265, 266, 267, 268, 269, 271, 273, 274, 275, 277, 278, 279, 280, 281, 283, 285, 290, 292, 293, 294, 296, 297, 300, 302, 303, 311, 312, 313, 314, 315, 316, 318, 324, 328, 336, 339, 341, 342, 343, 345, 346, 355, 358, 360, 364, 367, 368, 369, 370, 371, 372, 373, 374, 375, 377, 381, 382, 384, 386, 389, 391, 392, 401, 402, 405, 406, 409, 410, 411, 412, or 413, or combinations thereof, wherein the amino acid positions are relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the recombinant polynucleotide comprises a polynucleotide sequence encoding an engineered protease polypeptide comprising at least a substitution at amino acid position 135, 137, 139, 141, 143, 157, 160, 214, 268, 273, 279, 311, 312, 315, 328, 342, 345, 346, or 372, or combinations thereof, wherein the amino acid positions are relative to the reference sequence corresponding to residues 135-413 of SEQ ID NO: 948, 1126, 1368, or 1548, or relative to the reference sequence corresponding to SEQ ID NO: 948, 1126, 1368, or 1548.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 948, or to the reference sequence corresponding to SEQ ID NO: 948, wherein the amino acid sequence comprises one or more substitutions relative to the reference sequence corresponding to residues 135-413 of SEQ ID NO: 948, or relative to the reference sequence corresponding to SEQ ID NO: 948.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of an even-numbered SEQ ID NO. of SEQ ID NOs: 950-1154, or to the reference sequence corresponding to an even-numbered SEQ ID NO. of SEQ ID NOs: 950-1154, wherein the amino acid sequence comprises one or more substitutions relative to the reference sequence corresponding to residues 135-413 of SEQ ID NO: 948, or relative to the reference sequence corresponding to SEQ ID NO: 948.

In some embodiments, the recombinant polynucleotide comprises a polynucleotide sequence encoding an engineered protease polypeptide comprising an amino acid sequence comprising at least a substitution or substitution set at amino acid position(s) 411, 402, 285, 245, 266, 355, 258, 222, 140, 268, 225, 283, 406, 410, 143/145/243/312, 139/143/145/157/312, 139/157/345, 139/269, 156/157/342/346, 139/143, 269, 139/243/328, 269/328, 143/145/169, 328, 143/145/262, 145/262/312/328, 139/156/157, 139/145/312, 312, 139, 139/312, 139/156, 139/143/145/243, 145/157, 145/346, 145/262/312/328/345/346, 145/262, 312/342, 143/243, 139/345, 342, 143/145/262/342, 139/143/169, 139/143/145/312, 169, 139/145/262/312/328/342/345/346, 139/328, 139/243, 139/143/328, 139/143/243, 139/145, 145/312, 145/169, 139/143/157/312, 84/139/143, 145/269, 143/145/157/269/312/328, 143/145/269, 157, 139/143/312, 256, 273, 409, 172, 401, 281, 253, 143/145/243/328, 145, 139/143/145/328/342/345, 143/328/342/345, 145/342/345, 143, 139/145/328/342/345, 143/145/169/312/328/345/346, 143/243/328/342/345/346, 139/143/157/169/328/346, 143/145/156/312/328, 139/145/157/312/328, 143/328/342/345/346, 143/145, 143/145/312/342/345, or 143/145/328, wherein the amino acid positions are relative to the reference sequence corresponding to residues 135-413 of SEQ ID NO: 948, or relative to the reference sequence corresponding to SEQ ID NO: 948.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 1126, or to the reference sequence corresponding to SEQ ID NO: 1126, wherein the amino acid sequence comprises one or more substitutions relative to the reference sequence corresponding to residues 135-413 of SEQ ID NO: 1126, or relative to the reference sequence corresponding to SEQ ID NO: 1126.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of an even-numbered SEQ ID NO. of SEQ ID NOs: 1156-1422, or to the reference sequence corresponding to an even-numbered SEQ ID NO. of SEQ ID NOs: 1156-1422, wherein the amino acid sequence comprises one or more substitutions relative to the reference sequence corresponding to residues 135-413 of SEQ ID NO: 1126, or relative to the reference sequence corresponding to SEQ ID NO: 1126.

In some embodiments, the recombinant polynucleotide comprises a polynucleotide sequence encoding an engineered protease polypeptide comprising an amino acid sequence comprising at least a substitution or substitution set at amino acid position(s) 279, 250, 154, 214, 249, 275, 137, 161, 180, 174, 139, 254, 145, 278, 136, 154/413, 294, 237, 274, 264, 185, 277, 293, 233, 173, 312, 302, 238, 135, 221, 290, 263, 267, 239, 163, 292, 246, 243, 235, 156, 223, 278/413, 297, 194, 251, 253/411, 145/157/253/268/273/281/312/346/411, 139/346, 253, 346/411, 253/346, 312/346, 273/312, 253/281, 157/253/273/312/346/411, 139/157/253/268/273/281/312/346, 253/273/411, 139/253/268/273/281, 139/157/411, 157, 273, 139/253/268/273/281/312/411, 157/253/411, 139/145/253/346, 139/157/253/273/312, 139/157/268/273/312/346, 157/273/312/346, 139/411, 139/253/268/273/281/312/346/411, 157/273/346/411, 139/145/157/162/253/273/281/312, 139/253/273/281/312, 157/253/268/273/281/312, 139/253/268, 139/157/312, 253/273/281/346, 157/253/312/346/411, 157/273/312/346/411, 139/145/157/253/268/281/312, 139/273/312/346, 157/253/268/273/312/346, 139/268/346, 268/273/312/346, 139/157/253/268/273/312, 139/157/253, 139/253/281, 139/157/253/268/273, 253/312/411, or 139/268/273, wherein the amino acid positions are relative to the reference sequence corresponding to residues 135-413 of SEQ ID NO: 1126, or relative to the reference sequence corresponding to SEQ ID NO: 1126.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 1368, or to the reference sequence corresponding to SEQ ID NO: 1368, wherein the amino acid sequence comprises one or more substitutions relative to the reference sequence corresponding to residues 135-413 of SEQ ID NO: 1368, or relative to the reference sequence corresponding to SEQ ID NO: 1368.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of an even-numbered SEQ ID NO. of SEQ ID NOs: 1424-1608, or to the reference sequence corresponding to an even-numbered SEQ ID NO. of SEQ ID NOs: 1424-1608, wherein the amino acid sequence comprises one or more substitutions relative to the reference sequence corresponding to residues 135-413 of SEQ ID NO: 1368, or relative to the reference sequence corresponding to SEQ ID NO: 1368.

In some embodiments, the recombinant polynucleotide comprises a polynucleotide sequence encoding an engineered protease polypeptide comprising an amino acid sequence comprising at least a substitution or substitution set at amino acid position(s) 31, 318, 296, 252, 303, 253, 413, 386, 312, 235, 412, 342, 302, 371, 405, 389, 391, 358, 139/273/311/328/372, 311, 372, 139/143/157/160/268/273/311/315, 143, 139/143, 139/160/312/372, 143/273/328, 346, 135/139/160/268/312/342/346, 139/141/273, 135/141/143/268/273/312/372, 139/141/143/311, 139/157/268/328/346/372, 53/139/141/143/273/372, 139, 139/141/143/273/312, 137/139/221/233/413, 233, 221/279, 137/139/233/279, 221, 139/214, 137/139/156, 139/214/221, 214/233, 137/139/221/233/279, 137/139/221, 137/139, 137/139/279, 137, 137/139/214/279, 266, 139/221, 137/221, 137/156/214/312, 137/221/233, 137/139/156/221, 137/139/214, 279, 137/139/233, 137/139/214/233, 221/413, 137/214/233, 137/156, 137/139/221/233, 214/221, 137/221/413, 214, 137/233, 137/413, 137/221/279, 137/139/156/214/233/413, or 137/139/156/214, wherein the amino acid positions are relative to the reference sequence corresponding to residues 135-413 of SEQ ID NO: 1368, or relative to the reference sequence corresponding to SEQ ID NO: 1368.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 1548, or to the reference sequence corresponding to SEQ ID NO: 1548, wherein the amino acid sequence comprises one or more substitutions relative to the reference sequence corresponding to residues 135-413 of SEQ ID NO: 1548, or relative to the reference sequence corresponding to SEQ ID NO: 1548.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of an even-numbered SEQ ID NO. of SEQ ID NOs: 1610-1710, or to the reference sequence corresponding to an even-numbered SEQ ID NO. of SEQ ID NOs: 1610-1710, wherein the amino acid sequence comprises one or more substitutions relative to the reference sequence corresponding to residues 135-413 of SEQ ID NO: 1548, or relative to the reference sequence corresponding to SEQ ID NO: 1548.

In some embodiments, the recombinant polynucleotide comprises a polynucleotide sequence encoding an engineered protease polypeptide comprising an amino acid sequence comprising at least a substitution or substitution set at amino acid position(s) 145/273/372, 145/256/273, 221/243/273/328/372, 372, 145/221/279/372/406, 273/328, 145/169/273/346/406, 256/273, 145/221/273/328/346/406, 243/273, 145/214/256/273/279/328/372, 169/221/328/372/406, 372/406, 169/328/372/406, 221/372, 145/221/273/328/372, 214/243/273/328, 145/221, 214/256/273/346/372, 221/406, 169/372, 145/214/221/273, 145/221/346/372, 243/273/328/372/406, 145/169/273/328/346, 328, 169/273/372, 145/221/328, 169/214/273, 221/273/328, 221, 145/328, 214/346, 312, 212, 279, 212/312, 145, 179/346, 214, 346, 315/372, 375, 264, 179, 185, 220/372, or 324, wherein the amino acid positions are relative to the reference sequence corresponding to residues 135-413 of SEQ ID NO: 1548, or relative to the reference sequence corresponding to SEQ ID NO: 1548.

In some embodiments, the recombinant polynucleotide comprises a polynucleotide sequence encoding an engineered protease polypeptide comprising an amino acid sequence comprising at least a substitution at an amino acid position set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the recombinant polynucleotide comprises a polynucleotide sequence encoding an engineered protease polypeptide comprising an amino acid sequence comprising at least a substitution as set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the recombinant polynucleotide comprises a polynucleotide sequence encoding an engineered protease polypeptide comprising an amino acid sequence comprising at least a substitution or substitution set at amino acid position(s) set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the recombinant polynucleotide comprises a polynucleotide sequence encoding an engineered protease polypeptide comprising at least a substitution or substitution set of an engineered protease polypeptide variant set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the recombinant polynucleotide comprises a polynucleotide sequence encoding an engineered protease polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence having a substitution or substitution set as set forth in Tables 3.1, 3.2, 4.1, 4.2, 5.1, 5.2, 6.1, 7.1, 8.1, and 9.1, wherein the amino acid positions are relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

In some embodiments, the recombinant polynucleotide comprises a polynucleotide sequence encoding an engineered protease polypeptide comprising an amino acid sequence comprising residues 135-413 of an even-numbered SEQ ID NO. of SEQ ID NOs: 6-2242, or an amino acid sequence comprising an even-numbered SEQ ID NO. of SEQ ID NOs: 6-2242.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference polynucleotide sequence corresponding to nucleotide residues 403 to 1239 of SEQ ID NO: 3, 627, 947, 1125, 1367, or 1547, or to a reference polynucleotide sequence corresponding to SEQ ID NO: 3, 627, 947, 1125, 1367, or 1547, wherein the recombinant polynucleotide encodes an engineered protease polypeptide.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference polynucleotide sequence corresponding to nucleotide residues 403-1239 of an odd-numbered SEQ ID NO. of SEQ ID NOs: 5-2241, or to a reference polynucleotide corresponding to an odd-numbered SEQ ID NO. of SEQ ID NOs: 5-2241, wherein the recombinant polynucleotide encodes an engineered protease polypeptide.

As discussed herein, the polynucleotide sequence of the recombinant polynucleotide encoding an engineered protease polypeptide is codon optimized. In some embodiments, the polynucleotide sequence is codon optimized for expression in a selected host cell. In some embodiments, the polynucleotide sequence is codon optimized for expression in a bacterial cell, fungal cell, insect cell, or mammalian cell.

In some embodiments, the recombinant polynucleotide comprises a polynucleotide sequence comprising residues 403-1239, or residues 382-1239 of SEQ ID NO: 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 7299, 731, 733, 735, 737, 739, 741, 743, 745, 7437, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1471, 1473, 1475, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, 1617, 1619, 1621, 1623, 1625, 1627, 1629, 1631, 1633, 1635, 1637, 1639, 1641, 1643, 1645, 1647, 1649, 1651, 1653, 1655, 1657, 1659, 1661, 1663, 1665, 1667, 1669, 1671, 1673, 1675, 1677, 1679, 1681, 1683, 1685, 1687, 1689, 1691, 1693, 1695, 1697, 1699, 1701, 1703, 1705, 1707, 1709, 1711, 1713, 1715, 1717, 1719, 1721, 1723, 1725, 1727, 1729, 1731, 1733, 1735, 1737, 1739, 1741, 1743, 1745, 1747, 1749, 1751, 1753, 1755, 1757, 1759, 1761, 1763, 1765, 1767, 1769, 1771, 1773, 1775, 1777, 1779, 1781, 1783, 1785, 1787, 1789, 1791, 1793, 1795, 1797, 1799, 1801, 1803, 1805, 1807, 1809, 1811, 1813, 1815, 1817, 1819, 1821, 1823, 1825, 1827, 1829, 1831, 1833, 1835, 1837, 1839, 1841, 1843, 1845, 1847, 1849, 1851, 1853, 1855, 1857, 1859, 1861, 1863, 1865, 1867, 1869, 1871, 1873, 1875, 1877, 1879, 1881, 1883, 1885, 1887, 1889, 1891, 1893, 1895, 1897, 1899, 1901, 1903, 1905, 1907, 1909, 1911, 1913, 1915, 1917, 1919, 1921, 1923, 1925, 1927, 1929, 1931, 1933, 1935, 1937, 1939, 1941, 1943, 1945, 1947, 1949, 1951, 1953, 1955, 1957, 1959, 1961, 1963, 1965, 1967, 1969, 1971, 1973, 1975, 1977, 1979, 1981, 1983, 1985, 1987, 1989, 1991, 1993, 1995, 1997, 1999, 2001, 2003, 2005, 2007, 2009, 2011, 2013, 2015, 2017, 2019, 2021, 2023, 2025, 2027, 2029, 2031, 2033, 2035, 2037, 2039, 2041, 2043, 2045, 2047, 2049, 2051, 2053, 2055, 2057, 2059, 2061, 2063, 2065, 2067, 2069, 2071, 2073, 2075, 2077, 2079, 2081, 2083, 2085, 2087, 2089, 2091, 2093, 2095, 2097, 2099, 2101, 2103, 2105, 2107, 2109, 2111, 2113, 2115, 2117, 2119, 2121, 2123, 2125, 2127, 2129, 2131, 2133, 2135, 2137, 2139, 2141, 2143, 2145, 2147, 2149, 2151, 2153, 2155, 2157, 2159, 2161, 2163, 2165, 2167, 2169, 2171, 2173, 2175, 2177, 2179, 2181, 2183, 2185, 2187, 2189, 2191, 2193, 2195, 2197, 2199, 2201, 2203, 2205, 2207, 2209, 2211, 2213, 2215, 2217, 2219, 2221, 2223, 2225, 2227, 2229, 2231, 2233, 2235, 2237, 2239, or 2241.

In some embodiments, the recombinant polynucleotide comprises a polynucleotide sequence comprising SEQ ID NO: 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 7299, 731, 733, 735, 737, 739, 741, 743, 745, 7437, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1471, 1473, 1475, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, 1617, 1619, 1621, 1623, 1625, 1627, 1629, 1631, 1633, 1635, 1637, 1639, 1641, 1643, 1645, 1647, 1649, 1651, 1653, 1655, 1657, 1659, 1661, 1663, 1665, 1667, 1669, 1671, 1673, 1675, 1677, 1679, 1681, 1683, 1685, 1687, 1689, 1691, 1693, 1695, 1697, 1699, 1701, 1703, 1705, 1707, 1709, 1711, 1713, 1715, 1717, 1719, 1721, 1723, 1725, 1727, 1729, 1731, 1733, 1735, 1737, 1739, 1741, 1743, 1745, 1747, 1749, 1751, 1753, 1755, 1757, 1759, 1761, 1763, 1765, 1767, 1769, 1771, 1773, 1775, 1777, 1779, 1781, 1783, 1785, 1787, 1789, 1791, 1793, 1795, 1797, 1799, 1801, 1803, 1805, 1807, 1809, 1811, 1813, 1815, 1817, 1819, 1821, 1823, 1825, 1827, 1829, 1831, 1833, 1835, 1837, 1839, 1841, 1843, 1845, 1847, 1849, 1851, 1853, 1855, 1857, 1859, 1861, 1863, 1865, 1867, 1869, 1871, 1873, 1875, 1877, 1879, 1881, 1883, 1885, 1887, 1889, 1891, 1893, 1895, 1897, 1899, 1901, 1903, 1905, 1907, 1909, 1911, 1913, 1915, 1917, 1919, 1921, 1923, 1925, 1927, 1929, 1931, 1933, 1935, 1937, 1939, 1941, 1943, 1945, 1947, 1949, 1951, 1953, 1955, 1957, 1959, 1961, 1963, 1965, 1967, 1969, 1971, 1973, 1975, 1977, 1979, 1981, 1983, 1985, 1987, 1989, 1991, 1993, 1995, 1997, 1999, 2001, 2003, 2005, 2007, 2009, 2011, 2013, 2015, 2017, 2019, 2021, 2023, 2025, 2027, 2029, 2031, 2033, 2035, 2037, 2039, 2041, 2043, 2045, 2047, 2049, 2051, 2053, 2055, 2057, 2059, 2061, 2063, 2065, 2067, 2069, 2071, 2073, 2075, 2077, 2079, 2081, 2083, 2085, 2087, 2089, 2091, 2093, 2095, 2097, 2099, 2101, 2103, 2105, 2107, 2109, 2111, 2113, 2115, 2117, 2119, 2121, 2123, 2125, 2127, 2129, 2131, 2133, 2135, 2137, 2139, 2141, 2143, 2145, 2147, 2149, 2151, 2153, 2155, 2157, 2159, 2161, 2163, 2165, 2167, 2169, 2171, 2173, 2175, 2177, 2179, 2181, 2183, 2185, 2187, 2189, 2191, 2193, 2195, 2197, 2199, 2201, 2203, 2205, 2207, 2209, 2211, 2213, 2215, 2217, 2219, 2221, 2223, 2225, 2227, 2229, 2231, 2233, 2235, 2237, 2239, or 2241.

In some embodiments, the recombinant polynucleotide comprises a polynucleotide sequence comprising residues 403-1239, or residues 382-1239 of SEQ ID NO: 627, 947, 1125, 1367, 1547, 1639, or 1709, or a polynucleotide sequence comprising SEQ ID NO: 627, 947, 1125, 1367, 1547, 1639, or 1709.

In some embodiments, the present disclosure provides a recombinant polynucleotide capable of hybridizing under highly stringent conditions to a reference polynucleotide encoding an engineered protease polypeptide described herein, e.g., a recombinant polynucleotide provided in Tables 3.1, 3.2, 4.1, 4.2, 5.1, 5.2, 6.1, 7.1, 8.1, and 9.1, or a reverse complement thereof. In some embodiments, the present disclosure provides a recombinant polynucleotide capable of hybridizing under highly stringent conditions to a reverse complement of a reference polynucleotide encoding an engineered protease polypeptide described herein, wherein the recombinant polynucleotide hybridizing under stringent conditions encodes an protease polypeptide comprising an amino acid sequence having one or more residue differences as compared to SEQ ID NO: 4, 628, 948, 1126, 1368, or 1548, at residue positions selected from any positions as set forth in Tables 3.1, 3.2, 4.1, 4.2, 5.1, 5.2, 6.1, 7.1, 8.1, and 9.1. In some embodiments, the recombinant polynucleotide that hybridizes under highly stringent conditions comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 403-1239, or residues 382-1239 of SEQ ID NO: 3, 627, 947, 1125, 1367, or 1547, or to a reference polynucleotide sequence corresponding to SEQ ID NO: 3, 627, 947, 1125, 1367, or 1547. In some additional embodiments, the polynucleotide hybridizing under highly stringent conditions comprises a polynucleotide sequence having at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to at least one polynucleotide reference sequence corresponding to nucleotide residues 403-1239, or residues 382-1239 of a polynucleotide sequence provided in Tables 3.1, 3.2, 4.1, 4.2, 5.1, 5.2, 6.1, 7.1, 8.1, and 9.1, or a polynucleotide sequence provided in Tables 3.1, 3.2, 4.1, 4.2, 5.1, 5.2, 6.1, 7.1, 8.1, and 9.1, wherein the recombinant polynucleotide hybridizing under stringent conditions encodes an engineered protease polypeptide.

In some embodiments, the recombinant polynucleotide comprises a polynucleotide sequence encoding an engineered protease polypeptide comprising a signal sequence or a signal peptide, as described herein. In some embodiments, the encoded signal sequence or signal peptide is functional in the host cell used or to be used for expression of the engineered protease polypeptide. In some embodiments the encoded signal sequence or signal peptide is fused to a pro-polypeptide form of the engineered protease to form a pre-pro-polypeptide. In some embodiments, the encoded signal sequence or signal peptide is fused to the polypeptide that includes the mature, active form of the engineered protease. In some embodiments, the encoded signal sequence can be a naturally occurring signal sequence or a synthetic signal sequence, including a hybrid signal sequence.

In some embodiments, the recombinant polynucleotide comprises a polynucleotide sequence encoding an engineered protease polypeptide comprising a fusion polypeptide. In some embodiments, the encoded engineered protease polypeptide can be fused to a variety of polypeptide sequences as described above. In some embodiments, the encoded fusion polypeptide of the engineered protease polypeptides comprises a glycine-histidine or histidine-tag (His-tag), such as provided at the carboxy terminal region of SEQ ID NO: 2. In some embodiments, the encoded fusion protein of the engineered protease polypeptides comprise an epitope tag, such as c-myc, FLAG, V5, or hemagglutinin (HA). In some embodiments, the fusion protein of the engineered protease polypeptides comprises a GST, SUMO, Strep, MBP, or GFP tag.

In another aspect, the present disclosure further provides an expression vector comprising a recombinant polynucleotide encoding an engineered protease polypeptide described herein, e.g., for expression of the encoded engineered protease polypeptide. In some embodiments, the expression vector comprises one or more control sequences operably linked to the recombinant polynucleotide to regulate the expression of the recombinant polynucleotide and/or encoded polypeptide. In some embodiments, the control sequences include, among others, promoters, leader sequences, polyadenylation sequences, pro-peptide sequences, signal peptide sequences, and transcription terminators. In some embodiments, the control sequences, such as promoters, leader sequences, polyadenylation sequences, pro-peptide sequences, signal peptide sequences, and transcription terminators, are selected depending on the type chosen host cell into which the expression vector is to be introduced.

In some embodiments, suitable promoters are selected based on the host cells. In some embodiments, the promoter is a heterologous promoter. In some embodiments, for bacterial host cells, suitable promoters include, among others, promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (see, e.g., Villa-Kamaroff et al., Proc. Natl Acad. Sci. USA, 1978, 75:3727-3731), as well as the tac promoter (see, e.g., DeBoer et al., Proc. Natl Acad. Sci. USA, 1983, 80:21-25). In some embodiments, for fungal host cells, suitable promoters include, among others, promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (see, e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. In some embodiments, promoters effective in Pichia cells are used. In some embodiments, for insect host cells, suitable promoters include, among others, baculovirus promoters (e.g., P10 and polyhedron promoters), OpIE2 promoter, and Nephotettix cincticeps actin promoters. In some embodiments, for mammalian host cells, suitable promoters include, among others, promoters of cytomegalovirus (CMV), chicken β-actin promoter fused with the CMV enhancer, simian virus 40 (SV40), human phosphoglycerate kinase, beta actin, elongation factor-la or glyceraldehyde-3-phosphate dehydrogenase, or Gallus β-actin.

In some embodiments, the control sequence is a suitable transcription terminator sequence (i.e., a sequence recognized by a host cell to terminate transcription). In some embodiments, the terminator sequence is operably linked to the 3′ terminus of the nucleic acid sequence encoding the engineered protease polypeptide. Any suitable terminator which is functional in the host cell of choice finds use for the purposes in the present disclosure. In some embodiments, for bacterial expression, suitable transcription terminators include, among others, Rho-dependent terminators that rely on a Rho transcription factor, or Rho-independent, or intrinsic terminators, which do not require a transcription factor. Exemplary bacterial transcription terminators are described in Peters et al., J Mol Biol., 2011, 412(5):793-813. In some embodiments, for fungal host cells, suitable transcription terminators include, among others, terminators from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (see, e.g., Romanos et al., supra). In some embodiments, for mammalian host cells, suitable terminators include, among others, transcription terminators of cytomegalovirus (CMV), Simian virus 40 (SV40), human growth hormone hGH, bovine growth hormone BGH, and human or rabbit beta globulin.

In some embodiments, the control sequence is also a suitable leader sequence (i.e., a non-translated region of an mRNA that is important for translation by the host cell). In some embodiments, the leader sequence is operably linked to the 5′ terminus of the nucleic acid sequence encoding the engineered protease polypeptide. Any suitable leader sequence that is functional in the host cell of choice find use in expression of the engineered protease polypeptide. Exemplary leader sequences for mammalian and insect cells include, among others, leader sequences of expressed genes (e.g., heat shock protein, myosin, BIP immunoglobulin binding protein, GRP glucose regulated protein, etc.), viral leader sequences (e.g., EMC virus) and synthetic leader sequences, e.g., hTEE-658 and those described in, for example Cao et al., Nature Commun., 2021, 12:4138, incorporated herein by reference. Exemplary leader sequences for fungal expression include, among others, those from Aspergillus oryzae TAKA amylase, and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

In some embodiments, the control sequence is also a polyadenylation sequence (i.e., a sequence operably linked to the 3′ terminus of the nucleic acid sequence and which, when transcribed, is recognized by the host cell as a signal to add polyadenosine residues to transcribed mRNA). Any suitable polyadenylation sequence which is functional in the host cell of choice finds use in the present invention. Exemplary polyadenylation sequences for mammalian and insect cells include, among others, those of genes for human and mouse alpha-globin, mouse kappa light chain, chicken ovalbumin, SV40, as wells a synthetic polyA sequences (see, e.g., Clerici et al., eLife, 2017, 6:e33111). Exemplary polyadenylation sequences for fungal host cells include, but not limited to, the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are known in the art (see, e.g., Guo and Sherman, Mol. Cell. Bio., 1995, 15:5983-5990).

In some embodiments, the control sequence comprises one or more regulatory sequences that facilitate regulation of the expression of the polynucleotide and/or corresponding encoded polypeptide relative to the growth of the host cell. Examples of regulatory systems are those that cause the expression of the gene to be turned on or off in response to a chemical or physical stimulus, including the presence of a regulatory compound. In prokaryotic host cells, suitable regulatory sequences include, among others, the lac, tac, and trp operator systems. In yeast host cells, suitable regulatory systems include, among others the ADH2 system or GAL1 system. In filamentous fungi, suitable regulatory sequences include, among others, the TAKA alpha-amylase promoter, Aspergillus niger glucoamylase promoter, and Aspergillus oryzae glucoamylase promoter. In mammalian cells, suitable regulatory systems include, among others, zinc-inducible sheep metallothionine (MT) promoter, dexamethasone (Dex)-inducible promoter, mouse mammary tumor virus (MMTV) promoter; ecdysone insect promoter, tetracycline-inducible promoter system, RU486-inducible promoter system, and the rapamycin-inducible promoter system.

In some embodiments, the recombinant expression vector may be any suitable vector (e.g., a plasmid or virus), that can be conveniently subjected to recombinant DNA procedures and bring about the expression of the protease-encoding polynucleotide. The choice of the vector typically depends on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector is one in which, when introduced into the host cell, it is integrated into the genome and replicated together with the chromosome(s) into which it has been integrated. Furthermore, in some embodiments, a single vector or plasmid, or two or more vectors or plasmids which together contain the total DNA to be introduced into the genome of the host cell, and/or a transposon is utilized.

In some embodiment, recombinant polynucleotides may be provided on a non-replicating expression vector or plasmid. In some embodiments, the non-replicating expression vector or plasmid can be based on viral vectors defective in replication (see, e.g., Travieso et al., Vaccines, 2022, Vol. 7, Article 75).

In some embodiments, the expression vector contains one or more selectable markers, which permit selection of transformed cells. A “selectable marker” is a gene, the product of which provides for biocide or viral resistance, resistance to heavy metals, prototrophy to auxotrophs, and the like. Examples of bacterial selectable markers include, among others, the dal genes from Bacillus subtilis or Bacillus licheniformis, or markers, which confer antibiotic resistance such as ampicillin, kanamycin, chloramphenicol, or tetracycline resistance. Suitable markers for yeast host cells include, among others, ADE2, HIS3, LEU2, LYS2, MET3, TRP1, and URA3. Selectable markers for use in filamentous fungal host cells include, among others, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof. Selectable marker for mammalian cells include, among others, chloramphenicol acetyl transferase (CAT), nourseothricin N-acetyl transferase, blasticidin-S deaminase, blastcidin S acetyltransferase, Sh ble (Zeocin® resistance), aminoglycoside 3′-phosphotransferase (neomycin resistance), hph (hygromycin resistance), thymidine kinase, and puromycin N-acetyl-transferase.

In another aspect, the present disclosure provides a host cell comprising a recombinant polynucleotide encoding an engineered protease polypeptide described herein, the polynucleotide(s) being operably linked to one or more control sequences for expression of the encoded engineered protease polypeptide(s) in the host cell. In some embodiments, the host cell is a bacterial cell, fungal cell, insect cell, or mammalian cell.

In some embodiments, the host cell is a bacterial cell, including, among others, E. coli, B. subtilis, Vibrio fluvialis, Streptomyces and Salmonella typhimurium cell. Exemplary bacterial host cells also include various Escherichia coli strains (e.g., W3110 (ΔfhuA) and BL21). In some embodiments, the host cell is a fungal cell, such as filamentous fungal cell or yeast cell. n some embodiments, suitable fungal host cells include, among others, Pichia, Saccharomyces, Yarrowia, Kluyveromyces, Aspergillus, Trichoderma, Neurospora, Mucor, Penicillium T. Trichoderma, or Myceliophthora fungal cell. Exemplary fungal host cell includes, among others, Pichia pastoris, Yarrowia lipolytica, Kluyveromyces marxianus, Kluyveromyces lactis, Aspergillus niger, Aspergillus oryzae, Aspergillus fumigatus Trichoderma reesei. Neurospora crassa, Mucor circinelloides, Penicillium chrysogenum T. reesei, Trichoderma harzianum, Saccharomyces cerevisiae, or Myceliophthora thermophile. In some embodiments, the host cell is an insect cell. In some embodiments, a suitable insect host cell is a lepidopteran or dipteran insect cell. Exemplary insect host cell includes, among others, Sf9 cell, Sf21 cell, Schneider 2 cell, and BTI-TN-5B1-4 (High Five) cell. In some embodiments, the host cell is a mammalian cell. In some embodiments, the host cell is a human cell or rodent cell. Exemplary mammalian cells include, among others, Expi293, HeLa, U2OS, A549, HT1080, CAD, P19, NIH 3T3, L929, Hek 293, 293F, 293E, 293T, COS, Vero, NS0, Sp2/0 cell, DUKX-X11, MCF-7, Y79, SO-Rb50, Hep G2, J558L, and CHO cell.

In some embodiments, the host cell expresses an engineered protease polypeptide described herein. In some embodiments, the engineered protease polypeptide expressed in the host cell is a pre-pro-polypeptide or pre-pro-enzyme form of the engineered protease polypeptide. In some embodiments, the engineered protease polypeptide expressed in the host cell is a pro-polypeptide or pro-enzyme form of an engineered protease polypeptide. In some embodiments, the engineered protease polypeptide expressed in the host cell is a proteolytically active polypeptide or an active protease of the engineered protease polypeptide. In some embodiments, the engineered protease polypeptide expressed in the host cell is mature, active protease of the engineered protease polypeptide.

In some embodiments, any suitable method for introducing polynucleotides for expression of the engineered protease polypeptides into cells will find use for the purposes herein. Suitable techniques include, among others electroporation, biolistic particle bombardment, liposome mediated transfection, calcium chloride transfection, and protoplast fusion.

In some embodiments, recombinant polynucleotides encoding the engineered protease polypeptide can be produced using any suitable methods known the art. For example, a wide variety of different mutagenesis techniques are available to the skilled artisan. Methods are available to make specific substitutions at defined amino acids (site-directed), specific or random mutations in a localized region of the gene (region-specific), or random mutagenesis over the entire gene (e.g., saturation mutagenesis). Numerous methods known to those in the art to generate polypeptide variants, include, by way of example and not limitation, site-directed mutagenesis of single-stranded DNA or double-stranded DNA using PCR, cassette mutagenesis, gene synthesis, error-prone PCR, shuffling, and chemical saturation mutagenesis. Non-limiting examples of methods used for DNA and protein engineering are provided in the following references: U.S. Pat. Nos. 6,117,679; 6,420,175; 6,376,246; 6,586,182; 7,747,391; 7,747,393; 7,783,428; and 8,383,346. After the variants are produced, they can be screened for any desired property (e.g., increased activity, increased thermal activity, increased stability, increased thermostability, increased resistance to gastric proteases, increased pH stability, etc.).

In some embodiments, the engineered protease polypeptides with the properties disclosed herein can be obtained by subjecting the polynucleotide encoding the naturally occurring or engineered protease polypeptide to a suitable mutagenesis and/or directed evolution methods known in the art, such as provided in the Examples. An exemplary directed evolution technique is mutagenesis and/or DNA shuffling (see, e.g., Stemmer, Proc. Natl. Acad. Sci. USA, 1994, 91:10747-10751; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (see, e.g., Zhao et al., Nat. Biotechnol., 1998, 16:258-261), mutagenic PCR (see, e.g., Caldwell et al., PCR Methods Appl., 1994, 3:S136-S140), and cassette mutagenesis (see, e.g., Black et al., Proc. Natl. Acad. Sci. USA, 1996, 93:3525-3529).

Guidance for other suitable mutagenesis and directed evolution methods are described in, among others, U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 9,593,326, 9,665,694, 9,684,771, and all related PCT and non-US counterparts; Ling et al., Anal. Biochem., 1997, 254(2):157-78; Dale et al., Meth. Mol. Biol., 1996, 57:369-74; Smith, Ann. Rev. Genet., 1985, 19:423-462; Botstein et al., Science, 1985, 229:1193-1201; Carter, Biochem. J., 1986, 237:1-7; Kramer et al., Cell, 1984, 38:879-887; Wells et al., Gene, 1985, 34:315-323; Minshull et al., Curr. Op. Chem. Biol., 1999, 3:284-290; Christians et al., Nat. Biotechnol., 1999, 17:259-264; Crameri et al., Nature, 1998, 391:288-291; Crameri, et al., Nat. Biotechnol., 1997, 15:436-438; Zhang et al., Proc. Nat. Acad. Sci. U.S.A., 1997, 94:4504-4509; Crameri et al., Nat. Biotechnol., 1996, 14:315-319; Stemmer, Nature, 1994, 366:389-391; Stemmer, Proc. Nat. Acad. Sci. USA, 1994, 91:10747-10751; EP 3 049 973; WO 95/22625; WO 97/0078; WO 97/35966; WO 98/27230; WO 00/42651; WO 01/75767; WO 2009/152336; and WO 2015/048573, all of which are incorporated herein by reference.

In some embodiments, the clones obtained following mutagenesis treatment are screened by subjecting polypeptide preparations to a defined treatment conditions or assay conditions (e.g., temperature, pH condition, gastric protease, etc.) and measuring polypeptide activity after the treatments or other suitable assay conditions. Clones containing a polynucleotide encoding the polypeptide of interest are then isolated, the polynucleotide sequenced to identify the nucleotide sequence changes (if any), and used to express the polypeptide in a host cell. Measuring polypeptide activity from the expression libraries can be performed using any suitable method known in the art and as described in the Examples.

For engineered polypeptides of known sequence, the polynucleotides encoding the subject polypeptide can be prepared by standard solid-phase methods, according to known synthetic methods. In some embodiments, fragments of up to about 100 bases can be individually synthesized, then joined (e.g., by enzymatic or chemical ligation methods, or polymerase mediated methods) to form any desired continuous sequence. For example, polynucleotides and oligonucleotides disclosed herein can be prepared by chemical synthesis using the classical phosphoramidite method (see, e.g., Beaucage et al., Tet. Lett., 1981, 22:1859-69; and Matthes et al., EMBO J., 1984, 3:801-05), as it is typically practiced in automated synthetic methods. According to the phosphoramidite method, oligonucleotides are synthesized, e.g., in an automatic DNA synthesizer, purified, annealed, ligated, and cloned in appropriate vectors.

In some embodiments, a method for preparing the engineered protease polypeptide can comprise: (a) synthesizing a polynucleotide encoding a polypeptide comprising an amino acid sequence selected from the amino acid sequence of any engineered protease polypeptide as described herein, and (b) expressing the engineered protease polypeptide encoded by the polynucleotide. In some embodiments of the method, the amino acid sequence encoded by the polynucleotide can optionally have one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions are conservative or non-conservative substitutions.

### Methods of Preparing Engineered Protease Polypeptide and Proteolytically Active Polypeptide

In another aspect, the present disclosure provides a method of producing an engineered protease polypeptide, where the method comprises culturing a host cell comprising an expression vector capable of expressing a polynucleotide encoding the engineered protease polypeptide under suitable conditions such that the engineered protease polypeptide is expressed or produced. Appropriate culture media and growth conditions for various host cells are known in the art.

In some embodiments, the method further comprises a step of isolating the expressed protease polypeptide, such as from the culture medium and/or cells. In some embodiments, the method further comprises purifying the expressed engineered protease polypeptide. In some embodiments, isolating and/or purifying the engineered protease polypeptide can be done by using any one or more of the known techniques for protein purification, including, among others, detergent lysis, sonication, filtration, salting-out, selective precipitation, ultra-centrifugation, and chromatography.

Chromatographic techniques for isolation and/or purification of polypeptides and proteins include, among others, reverse phase chromatography, high-performance liquid chromatography, ion-exchange chromatography, hydrophobic-interaction chromatography, size-exclusion chromatography, gel electrophoresis, and affinity chromatography. Conditions for purifying a particular polypeptide may depend, in part, on factors such as net charge, hydrophobicity, hydrophilicity, molecular weight, molecular shape, etc., and will be apparent to those having skill in the art. In some embodiments, affinity techniques may be used to isolate the engineered protease polypeptides. For affinity chromatography purification, any antibody that specifically binds the engineered protease polypeptide of interest can be used. Where the engineered protease includes a fusion polypeptide that includes an affinity tag, such as a His-tag, standard affinity methods for the particular fusion polypeptide can be used.

In some embodiments, the present disclosure provides a method of preparing a proteolytically active polypeptide or an active protease of an engineered protease polypeptide. In some embodiments, a method of preparing a proteolytically active protease polypeptide comprises incubating or reacting an engineered protease polypeptide described herein under suitable conditions such that the proteolytically active protease or active protease is produced. In some embodiments, the method is used to prepare a mature proteolytically active polypeptide or active protease of an engineered protease polypeptide.

In some embodiments, the proteolytically active polypeptide or active protease is prepared from an engineered protease polypeptide that contains a pro-domain of the protease. In some embodiments, the proteolytically active polypeptide or active protease is prepared from a pro-polypeptide form of the engineered protease polypeptide. In some embodiments, the proteolytically active polypeptide or active protease prepared by the method has an amino terminus within amino acid residues 128-135, particularly where the amino terminus is at amino acid residue 128 or 135, wherein the amino acid positions are numbered with respect to SEQ ID NO: 2, or an equivalent position for any engineered protease polypeptide variant described herein.

In some embodiments, the suitable conditions for preparing the proteolytically active polypeptide or active protease is sufficient for activation of autoproteolysis of the appropriate engineered protease polypeptide. Without being bound by any theory of operation, an engineered protease polypeptide containing a pro-domain can undergo auto-proteolysis to generate a proteolytically active polypeptide or an active protease in which autoproteolysis occurs at least at amino acid position 128 and/or 135, where the amino acid positions are numbered with respect to SEQ ID NO: 2, or an equivalent position for any of the engineered protease polypeptide variants described herein.

In some embodiments, the proteolytically active polypeptide or active protease can be prepared by subjecting the pro-polypeptide form of the engineered protease polypeptide to another protease that can cleave the pro-polypeptide and separate the pro-domain from the protease domain. In some embodiments, the other protease comprises a proteolytically active polypeptide or active protease of an engineered protease polypeptide described herein. Exemplary conditions for auto-proteolysis or proteolysis with an engineered protease are provided in the Examples.

### Compositions and Pharmaceutical Compositions

In a further aspect, the present disclosure provides a composition comprising an engineered protease polypeptide. In some embodiments, the composition comprises an engineered protease polypeptide, wherein the engineered protease polypeptide is in the form of a pre-pro-polypeptide or pre-pro-enzyme; a pro-polypeptide or pro-enzyme; or a proteolytically active polypeptide or active protease, including mature, proteolytically active polypeptide, as described herein. In some embodiments, the pro-polypeptide or pro-enzyme form of the engineered protease polypeptide if a form with reduced or low protease activity is desired, for example for storage and/or prior to activation of the protease.

In some embodiments, the composition comprises an engineered protease polypeptide as a dietary/nutritional supplement, or in combination with food or drink. In some embodiments, the engineered protease polypeptide may be used in any suitable edible enzyme delivery matrix. In some embodiments, engineered protease polypeptide are present in an edible enzyme delivery matrix designed for rapid dispersal of the protease within the digestive tract of an animal or subject upon ingestion of the polypeptide. In some embodiments, an engineered protease polypeptide is mixed or admixed with protein-containing food or a drink. Non-limiting examples of such foods include a protein-containing powder, a spread, a spray, a sauce, a dip, a cream, dressing, cheese, butter, margarines, spreads, butter, dairy products, nut butters, seed butters, kernel butters, peanut butter, vegetables, meats, poultry, and fish. In some embodiments, the engineered protease polypeptide is mixed or admixed with infant formula or with breast milk.

In some embodiments, the engineered protease is formulated as a pharmaceutical composition. Depending on the mode of administration, the compositions comprise a therapeutically effective amount of an engineered protease polypeptide and can be in the form of a solid, semi-solid, or liquid. In some embodiments, the pharmaceutical composition comprises an engineered protease polypeptide, and a pharmaceutically acceptable carrier, excipient, or diluent. The carrier can be a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers.

In some embodiments, the excipient includes, among others, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain appropriate amounts of wetting or emulsifying agents, and/or pH buffering agents. These compositions can take the form of solutions, suspensions, emulsion, tablets, pills, capsules, powders, sustained-release formulations and the like. Examples of suitable pharmaceutical carriers are described in Remington: The Science and Practice of Pharmacy, 23^{rd} Ed, A. Adejare ed., Academic Press, 2020, incorporated in its entirety by reference herein. Such compositions will contain a therapeutically effective amount of the engineered protease polypeptide, preferably in purified form, together with a suitable amount of carrier and/or excipient so as to provide the form for proper administration to the subject.

In some embodiments, the engineered protease polypeptide is formulated for use as oral pharmaceutical compositions (e.g., for oral administration). Any suitable format for use in delivering the protease polypeptide may be used, including but not limited to pills, tablets, gel tabs, capsules, lozenges, dragees, powders, soft gels, sol-gels, gels, emulsions, sprays, ointments, liniments, creams, pastes, jellies, demulcents, sticks, suspensions (including but not limited to oil-based suspensions, oil-in water emulsions, etc.), slurries, syrups, controlled release formulations. For oral administration, the pharmaceutical composition can be used alone or in combination with appropriate additives to make the tablets, powders, granules, capsules, syrups, liquids, suspensions, etc. For example, solid oral forms of the composition can be prepared with conventional additives, disintegrators, lubricants, diluents, buffering agents, moistening agents, preservatives and flavoring agents. Non-limiting examples of excipients include sugars (e.g., lactose, sucrose, mannitol, and/or sorbitol), starches (e.g., corn, wheat, rice, potato, or other plant starch), cellulose (e.g., methyl cellulose, hydroxypropylmethyl cellulose, sodium carboxy-methylcellulose), gums (e.g., arabic, tragacanth, guar, etc.), and/or proteins (e.g., gelatin, collagen, etc.). Additional components in oral formulations may include coloring and or sweetening agents (e.g., glucose, sucrose, and mannitol) and lubricating agents (e.g., magnesium stearate), as well as enteric coatings (e.g., methacrylate polymers, hydroxyl propyl methyl cellulose phthalate, and/or any other suitable enteric coating known in the art). In some embodiments, the formulation releases the enzyme(s) in the stomach of the subject so that target proteins can be degraded by the engineered protease.

In some embodiments, the engineered protease polypeptide is provided as a unit dose formulation. For example, and without limitation, the unit dose may be present in a tablet, a capsule, and the like. The unit dose may be in solid, liquid, powder, or any other form. A unit dose formulation of the pharmaceutical composition will allow for appropriate dosing while avoiding potential negative side effects of administering an excessive amount of the composition.

In some embodiments, the engineered protease polypeptide or composition thereof, including as a pharmaceutical composition, can be lyophilized from an aqueous solution, optionally in the presence of appropriate buffers (e.g., phosphate, citrate, histidine, imidazole buffers) and excipients (e.g., cryoprotectants such as sucrose, lactose, trehalose, etc.). Lyophilizates can optionally be blended with excipients and made into different forms.

In some embodiments, the composition, including a pharmaceutical composition, further comprises a lipase. In some embodiments, the lipase can be any lipase suitable for treating exocrine pancreatic insufficiency. In some embodiments, the composition, including a pharmaceutical composition further comprises an amylase. In some embodiments, the amylase can be any amylase suitable for treating exocrine pancreatic insufficiency. In some embodiments, the composition, including a pharmaceutical composition, further comprises a lipase and an amylase. In some embodiments, the protease and amylase are suitable for treating pancreatic insufficiency.

### Uses and Methods

In another aspect, the present disclosure provides use of the engineered protease polypeptides for degrading a target protein or polypeptide. In some embodiments, a method of degrading a target protein or polypeptide comprises contacting a target protein or polypeptide with an engineered protease polypeptide described under suitable conditions for proteolytically active polypeptide or an active protease of the engineered protease polypeptide to degrade the target protein or polypeptide. In some embodiments, the target protein or polypeptide comprises a mixture or proteins and/polypeptides. In some embodiments, the mixture of protein and/or polypeptides is protein(s) in food or drink.

In some embodiments, the engineered protease polypeptide is a pro-polypeptide form, and the suitable conditions promote formation of the proteolytically active polypeptide or active protease of the engineered protease polypeptide. In some embodiments, the engineered protease polypeptide is the proteolytically active protease or active protease, e.g., protease polypeptide comprising amino acid residues 135-413 or 128-413, that does not need any further activation.

In some embodiments, the engineered protease polypeptides are applied in the treatment of exocrine pancreatic insufficiency or a deficiency in pancreatic enzymes required for efficient digestion of proteins in food. In some embodiments, a method of treating a deficiency in pancreatic enzymes required for efficient digestion of proteins in food, comprises administering to a subject in need thereof an effective amount of an engineered protease polypeptide or a pharmaceutical composition thereof described herein. In some embodiments, the subject is a human patient with exocrine pancreatic insufficiency.

In some embodiments, the engineered protease polypeptide administered is the pro-polypeptide form, e.g., protease polypeptide of residues 1-413 of an engineered protease polypeptide, where the engineered protease polypeptide is activated to the proteolytically active polypeptide or active protease prior to administration, or administered under conditions that result in activation to the proteolytically active polypeptide or active protease. In some embodiments, the engineered protease polypeptide administered is the proteolytically active polypeptide or the active protease, e.g., protease polypeptide comprising residues 128-413 or 135-413 of an engineered protease polypeptide.

In some embodiments, the engineered protease polypeptide or pharmaceutical composition thereof is administered immediately prior to, concurrently with, or subsequent to consumption of a protein-containing food or drink. In some embodiments, the engineered protease polypeptide is preferably administered concurrently with the ingestion of the food or drink.

In some embodiments, the subject for treatment is a human infant. In some embodiments, the engineered protease polypeptide is administered with infant formula or during breast feeding.

In some embodiments, the subject for treatment is a child, e.g., 12 months or older and up to 4 years old. In some embodiments, the subject for treatment is a child older than 4 years old, or a young adult, up to 18 years of age. In some embodiments, the subject for treatment is a human adult.

In some embodiments, the present disclosure provides use of an engineered protease polypeptide described herein for treating exocrine pancreatic insufficiency.

In some embodiments, the present disclosure provides use of an engineered protease polypeptide described herein in the preparation of a medicament for treating exocrine pancreatic insufficiency.

### EXAMPLES

The following Examples, including experiments and results achieved, are provided for illustrative purposes only and are not to be construed as limiting the present invention. In the embodiments herein, the abbreviations and technical terms are those commonly used and known in the art.

### Example 1

### Protease Gene Acquisition and Construction of Expression Vectors

A wild-type protease (WP_077617485) of Bacillus sinesaloumensis Marseille P3516 from the serine peptidase S8 family with a C-terminal 6×-histidine tag (SEQ ID NO: 2) was codon optimized for expression in E. coli and cloned into an E. coli expression vector system (see, e.g., US Pat. Application WO2021/061915A2). In addition, in some embodiments, expression vectors lacking antimicrobial resistance markers are used. The plasmid construct was transformed into an E. coli strain derived from W3110. Directed evolution techniques were used to generate libraries of gene variants from this plasmid construct (see e.g., U.S. Pat. No. 8,383,346, and WO2010/144103) as well as its derivatives.

### Example 2

### High-Throughput (HTP) Growth of Protease Variants and Screening Conditions

2.1: HTP Growth of Bacillus sinesaloumensis Marseille P3516 Protease and Variants

Transformed E. coli cells were selected by plating onto Luria Broth (LB) agar plates containing 1% glucose with selection. After overnight incubation at 37° C., colonies were placed into the wells of 96-well shallow flat bottom plates (NUNC™, Thermo-Scientific) filled with 180 μl/well LB supplemented with 1% glucose and selection (e.g., chloramphenicol). The cultures were allowed to grow overnight for 18-20 hours in a shaker (200 rpm, 30° C., and 85% relative humidity; Kuhner).

Overnight growth samples (20 μL) were transferred into Costar 96-well deep plates filled with 380 μL of Terrific Broth (TB) supplemented with a selection. The plates were incubated for 130 minutes in a shaker (250 rpm, 30° C., and 85% relative humidity; Kuhner). The cells were then induced with 40 μL of 10 mM isopropylthiogalactoside (IPTG) in sterile water and incubated overnight for 20-24 hours in a shaker (250 rpm, 30° C., and 85% relative humidity; Kuhner). The cells were pelleted (4000 rpm×20 min), the supernatants were discarded, and the cells were frozen at −80° C. prior to analysis.

### 2.2: Lysis of HTP Cell Pellets

For cell lysis, 200, 300, or 400 μL of lysis buffer (1×PBS, 1 mg/ml lysozyme, 0.5 mg/ml polymyxin B sulfate, 0.4 U/mL DNase I from New England Biolabs) was added to the cell pellets. The mixture was agitated for 1.5-2 hours at room temperature, and centrifuged (4000 rpm×15 min) prior to further analysis. At this stage, the sample is referred to as “clarified lysate.” Sometimes additional dilutions of clarified lysate were performed in PBS buffer prior to subsequent challenges and activity assays.

### 2.4: Activation of Clarified Lysates

To activate the protease, a small quantity of previously purified and activated protease was added to the clarified lysate to degrade the pro-peptide and facilitate the activation of the protease present in the lysate. Clarified lysate was mixed 1:1 with PBS containing 0.02 g/L of purified protease in a BioRad hardshell plate (final concentration 50% clarified lysate and 0.01 g/L purified protease). Samples were mixed and incubated at 37° C. in a thermocycler for 16+ hours. At this stage, the sample is referred to as “activated lysate.” Sometimes additional dilutions of activated lysate were performed in PBS buffer prior to subsequent challenges and activity assays.

2.5: Analysis of Activated Lysates for Protease Activity with Casein Activity Assay

The activity of protease variants was determined by measuring the degradation of Casein using an activity assay adapted for high throughput from the United States Pharmacopeia protease assay. For this assay, 80 μL of reaction buffer (50 mM Potassium Phosphate buffer, pH 7.5, or 100 mM sodium phosphate buffer, pH 6.5) was added to a Costar deepwell plate. Next, 80 μL of 15 g/L casein sodium salt dissolved in water was added to the reaction plate. Finally, 40 μL of the sample to be analyzed was added to the reaction plate, starting the reaction. Reaction plates were incubated at 40° C. in a multitron shaker with shaking at 400 rpm for 1 hour. After 1 hour, 200 μL of 50 g/L of trichloroacetic acid (TCA) was added to the reaction plate, simultaneously quenching the reaction and precipitating out any whole proteins. Quenched reactions were thoroughly shaken and centrifuged to pellet out any precipitated protein. After centrifugation, 200 μL of supernatant was transferred to a Greiner UV-star flat bottom plate and the absorbance was read at 280 nm using a Molecular Devices SpectraMax M2 plate reader. To ensure that samples did not saturate the assay, samples were often diluted prior to analysis based on a pre-determined dilution factor (up to 128×). The term “unchallenged activity” is defined as protease activity without any prior challenge described in Examples 2.7-2.8.

2.6: Analysis of Clarified Lysates for Protease Activity with BODIPY-Casein Assay

In some cases, the activity of protease variants was determined by measuring the degradation of Casein using the EnzChek Protease Assay Kit (Thermo Fisher Scientific). For this assay, 90 μL of 10 μg/mL BODIPY-casein substrate in aqueous buffer (100 mM sodium phosphate buffer, pH 7) was added to a 96-well, opaque, black microtiter plate (Costar). To start the assay, 10 μL of sample (challenged, diluted cell lysate) was added to the reaction mix and incubated at 37° C. in a multitron shaker with shaking at 400 rpm. After 1 hour, plates were read on a Molecular Devices SpectraMax M2 plate reader for fluorescence (Excitation: 485, Emission: 530). To ensure that samples did not saturate the assay, samples were diluted in assay buffer prior to analysis based on a pre-determined dilution factor (up to 250×). The term “unchallenged activity” is defined as protease activity without any prior challenge described in Examples 2.8.

2.7: HTP Analysis of Activated Lysates Pre-Incubated with Heat Challenge

Thermostability of protease variants were assessed as described herein. Clarified lysate or activated lysate was transferred to a PCR plate (BioRad) and incubated for 1 hour at 63° C., 64° C. or 71° C. in a thermocycler. After incubation, samples were centrifuged and the supernatant was analyzed for residual protease activity as described in Example 2.5.

### 2.8: HTP Analysis of Clarified Lysates or Activated Lysates Pre-Incubated at Reduced pH in the Presence of Pepsin

The activities of protease variants were determined after pre-incubation at low pH in the presence of pepsin to simulate the environment of the stomach. Clarified lysate, activated lysate, or activated lysate further diluted in PBS was mixed 1:1 with McIlvaine buffer, pH 2.8-4.5, +4000 U/mL (1.6 mg/mL) pepsin from porcine gastric mucosa (Sigma) in a PCR plate (BioRad), for a final challenge pH 2.8-4.5 and a final pepsin concentration of 2000 U/mL (0.8 mg/mL). Samples were mixed then incubated for 1 hour at 37° C. in a thermocycler. To stop the challenges, samples were mixed 1:1 with 400 mM sodium phosphate buffer, pH 7.0, neutralizing the pH and inactivating the pepsin. Neutralized challenge samples were then further diluted and analyzed for residual protease activity as described in Examples 2.5-2.6.

### Example 3

Screening Results of Protease Variants Derived from SEQ ID NO: 4
3.1: HTP Growth of Bacillus sinesaloumensis Marseille P3516 Protease and Variants

The polynucleotide sequence of SEQ ID NO: 1 encoding the polypeptide of SEQ ID NO: 2 was subcloned into a different E. coli expression vector system (see e.g., US Pat. Application WO2021061915A2) and was used as the backbone for the construction of protease variants in this Example. Within the Sequence Listing, for purposes of consistency in description, the protease variants studied in this Example are represented as a fragment (residues 1-413, SEQ ID NO: 4) of their full-length sequence of actual variants examined. In this Example, the actual variants studied include residues 414-533 of SEQ ID NO: 2 and the His-tag sequence (see FIG. 1). New variants were generated by known protein evolution techniques and the variants screened using the BODIPY-casein activity assay after a one hour pre-incubation at pH 4.5 in the presence of pepsin, as described in Example 2. Some variants were subjected to a modified pre-incubation with more lysate present in the challenge prior to screening with the BODIPY-casein activity assay. Analysis of the data relative to SEQ ID NO: 4 is listed in Table 3.1. Some variants from these libraries were assayed in triplicate in the BODIPY-casein activity assay after no prior challenge, and after a 1 hour pre-incubation at pH 4.5 in the presence of pepsin, as described in Example 2. Analysis of the average data relative to SEQ ID NO: 4 is listed in Table 3.2.

TABLE 3.1
Protease activity relative to SEQ ID NO: 4

| | | High lysate pH | |
|---|---|---|---|
| | | 4.5 and Pepsin | pH 4.5 and Pepsin |
| | Amino Acid | Challenge | Challenge |
| SEQ ID | Differences | Improvement | Improvement |
| NO: | (Relative to | Relative to | Relative to |
| (nt/aa) | SEQ ID NO: 4) | SEQ ID NO: 4 | SEQ ID NO: 4¹ |
| 3/4 | | + | + |
| 5/6 | N185F | ++ | |
| 7/8 | Q134I | ++ | |
| 9/10 | G129T | +++ | |
| 11/12 | A135C | ++ | |
| 13/14 | T184A | ++ | |
| 15/16 | G129R | +++ | |
| 17/18 | T132Y | ++ | |
| 19/20 | L186R | ++ | |
| 21/22 | G193T | + | |
| 23/24 | G263P | ++ | |
| 25/26 | D370C | + | |
| 27/28 | D45Y/Q134W | ++ | |
| 29/30 | N185M | + | |
| 31/32 | Q199K | ++ | |
| 33/34 | N368T | +++ | |
| 35/36 | S161E | +++ | |
| 37/38 | N141T | ++ | |
| 39/40 | Q267L | ++ | |
| 41/42 | A179S | ++ | |
| 43/44 | N185V | + | |
| 45/46 | G264L | +++ | |
| 47/48 | Q199C | + | |
| 49/50 | S160M | ++ | |
| 51/52 | P138Q | + | |
| 53/54 | A131Y | + | |
| 55/56 | T184D | + | |
| 57/58 | S372R | + | |
| 59/60 | Q134L | ++ | |
| 61/62 | D370R | ++ | |
| 63/64 | D370I | ++ | |
| 65/66 | Q134E | +++ | |
| 67/68 | N368G | ++ | |
| 69/70 | E151D | + | |
| 71/72 | Q274K | + | |
| 73/74 | Q134D | +++ | |
| 75/76 | Q134V | ++ | |
| 77/78 | E128I | ++ | |
| 79/80 | Y339S | +++ | |
| 81/82 | N313A | ++ | |
| 83/84 | A131E | + | |
| 85/86 | N185P | ++ | |
| 87/88 | A374W | + | |
| 89/90 | R314G | ++ | |
| 91/92 | V191R | + | |
| 93/94 | E128V | ++ | |
| 95/96 | T132E | ++ | |
| 97/98 | S324R | + | |
| 99/100 | T315M | + | |
| 101/102 | T132V | + | |
| 103/104 | Q375L | + | |
| 105/106 | Q375T | ++ | |
| 107/108 | G129L | + | |
| 109/110 | T132P | ++ | |
| 111/112 | T184M | + | |
| 113/114 | V136G | + | |
| 115/116 | L186A | + | |
| 117/118 | A135S | + | |
| 119/120 | Q220L | + | |
| 121/122 | Q134P | ++ | |
| 123/124 | T132A | + | |
| 125/126 | N141M | ++ | |
| 127/128 | A135I | + | |
| 129/130 | N194D | ++ | |
| 131/132 | N185Q | ++ | |
| 133/134 | G263H | ++ | |
| 135/136 | Q274L | ++ | |
| 137/138 | D231V | ++ | |
| 139/140 | T315R | ++ | |
| 141/142 | Q375S | ++ | |
| 143/144 | A135T | ++ | |
| 145/146 | N185G | ++ | |
| 147/148 | A135R | + | |
| 149/150 | V277D | + | |
| 151/152 | E128P | ++ | |
| 153/154 | T132R | + | |
| 155/156 | P369I | + | |
| 157/158 | G264C | ++ | |
| 159/160 | T315H | + | |
| 161/162 | I251S | + | |
| 163/164 | V136I | + | |
| 165/166 | S160P | ++ | |
| 167/168 | Q375I | ++ | |
| 169/170 | N180M | + | |
| 171/172 | P369V | ++ | |
| 173/174 | I251D | ++ | |
| 175/176 | G264A | + | |
| 177/178 | K163L | ++ | |
| 179/180 | D231H | ++ | |
| 181/182 | R343S | ++ | |
| 183/184 | G264R/Q279R | ++ | |
| 185/186 | Q274A | ++ | |
| 187/188 | Q279Y | ++ | |
| 189/190 | A131P | ++ | |
| 191/192 | N232S | ++ | |
| 193/194 | Q220R | ++ | |
| 195/196 | T315Q | + | |
| 197/198 | L186T | + | |
| 199/200 | S324V | ++ | |
| 201/202 | N313S | + | |
| 203/204 | T132D | ++ | |
| 205/206 | N141R/A300V | ++ | |
| 207/208 | S324I | ++ | |
| 209/210 | A367V | ++ | |
| 211/212 | A135V | ++ | |
| 213/214 | D370L | + | |
| 215/216 | T132G | ++ | |
| 217/218 | Q267G | ++ | |
| 219/220 | A131T | + | |
| 221/222 | N266T | + | |
| 223/224 | A179K | + | |
| 225/226 | S372A | + | |
| 227/228 | S372F | ++ | |
| 229/230 | N185T | ++ | |
| 231/232 | S324D | ++ | |
| 233/234 | A135K | + | |
| 235/236 | R188A | ++ | |
| 237/238 | N141D | ++ | |
| 239/240 | A374L | + | |
| 241/242 | N185D | ++ | |
| 243/244 | T130N | + | |
| 245/246 | D370V | + | |
| 247/248 | S161R | ++ | |
| 249/250 | T315I | + | |
| 251/252 | T315L | +++ | |
| 253/254 | S318N | ++ | |
| 255/256 | R188C | ++ | |
| 257/258 | N180L | ++ | |
| 259/260 | S372Y | ++ | |
| 261/262 | A135P | ++ | |
| 263/264 | Q375E | ++ | |
| 265/266 | S324A | ++ | |
| 267/268 | G129K | ++ | |
| 269/270 | Q134M | + | |
| 271/272 | T184G | +++ | |
| 273/274 | N185A | ++ | |
| 275/276 | G129H | ++ | |
| 277/278 | R188D | + | |
| 279/280 | T130F | + | |
| 281/282 | Y265C | ++ | |
| 283/284 | N141W | | ++ |
| 285/286 | S324W | | + |
| 287/288 | D370E | | ++ |
| 289/290 | T184R | | + |
| 291/292 | Q134A | | + |
| 293/294 | S161L | | ++ |
| 295/296 | Q134T | | + |
| 297/298 | D370G | | ++ |
| 299/300 | Q375A | | ++ |
| 301/302 | E128G | | ++ |
| 303/304 | T130V | | ++ |
| 305/306 | Q134N | | ++ |
| 307/308 | N341G | | ++ |
| 309/310 | F190S | | ++ |
| 311/312 | D370P | | + |
| 313/314 | N145R | | + |
| 315/316 | Q279H | | + |
| 317/318 | Q279S | | + |
| 319/320 | S160Q | | + |
| 321/322 | D370K | | + |
| 323/324 | A126T/G192C | | + |
| 325/326 | A374E | | ++ |
| 327/328 | E128K | | ++ |
| 329/330 | S160C | | + |
| 331/332 | L186S | | ++ |
| 333/334 | N11K/Q220K | | + |
| 335/336 | Q134W | | ++ |
| 337/338 | G129V | | ++ |
| 339/340 | E128L | | ++ |
| 341/342 | E151Q | | ++ |
| 343/344 | Q375M | | ++ |
| 345/346 | Q134C | | + |
| 347/348 | A374R | | ++ |
| 349/350 | S160T | | + |
| 351/352 | Q279T | | ++ |
| 353/354 | G264F | | ++ |
| 355/356 | T132C | | ++ |
| 357/358 | G129F | | ++ |
| 359/360 | G264V | | ++ |
| 361/362 | G129I | | ++ |
| 363/364 | T184Q | | + |
| 365/366 | G192M | | + |
| 367/368 | A374S | | ++ |
| 369/370 | D370F | | ++ |
| 371/372 | Q267A | | ++ |
| 373/374 | P369W | | + |
| 375/376 | Q199L | | + |
| 377/378 | N145M | | + |
| 379/380 | N194A | | ++ |
| 381/382 | N185S | | ++ |
| 383/384 | Y265R | | + |
| 385/386 | G129S | | ++ |
| 387/388 | N185R | | + |
| 389/390 | R188W | | ++ |
| 391/392 | S161G | ++ | |
| 393/394 | D370G/Q392Y | ++ | |
| 395/396 | I99V/A278N | ++ | |
| 397/398 | Y265G/T311D | ++ | |
| 399/400 | V84M/S159G/Y265G/ | ++ | |
| | Q279K/T311D/D370G | | |
| 401/402 | T311D/R316K | ++ | |
| 403/404 | S342N/D370G | ++ | |
| 405/406 | Y265G/T311D/D370G | ++ | |
| 407/408 | G192D/T311D/R316K | ++ | |
| 409/410 | N141Q/A154D/G192D | ++ | |
| 411/412 | Y265G/T311D/R316K/ | ++ | |
| | S342N | | |
| 413/414 | Q279K/T311D/R316K | ++ | |
| 415/416 | N141Q/Y265G/Q279K/ | ++ | |
| | T311D/S342N | | |
| 417/418 | N141Q/G192D/T311D/ | ++ | |
| | R316K/D370G | | |
| 419/420 | N141Q | ++ | |
| 421/422 | N141Q/Y265G/T311D | ++ | |
| 423/424 | V198G/Q279K | ++ | |
| 425/426 | Q392Y | + | |
| 427/428 | S342N/D370G/Q392Y | ++ | |
| 429/430 | N141Q/V198G/Y265G | ++ | |
| 431/432 | Y265G/Q392Y | ++ | |
| 433/434 | Y265G | ++ | |

All activities were determined relative to the reference polypeptide of SEQ ID NO: 4. Levels of increased activity are defined as follows: “+” 0.9 to 1.2, “++” >1.2, “+++” >1.8. The reference sequence and all variants within this table are represented in the Sequence Listing as a fragment (residues 1-413) of their full length sequence, as described in Example 3

TABLE 3.2
Protease activity relative to SEQ ID NO: 4

| | | pH 4.5 | |
|---|---|---|---|
| | | pepsin | Unchallenged |
| | Amino Acid | challenge | activity |
| SEQ | Differences | improvement | improvement |
| ID NO: | (Relative to | relative to | relative to |
| (nt/aa) | SEQ ID NO: 4) | SEQ ID NO: 4 | SEQ ID NO: 4 |
| 435/436 | A135L | + | |
| 437/438 | N194L | + | + |
| 439/440 | E128T | + | + |
| 441/442 | Q134S | + | + |
| 443/444 | N313T | + | + |
| 445/446 | T184L/Q267L | + | + |
| 447/448 | N185E | + | + |
| 449/450 | S342G | ++ | + |
| 451/452 | A374Y | + | + |
| 453/454 | N141R | + | |
| 455/456 | L186Y | + | + |
| 457/458 | S312R | + | |
| 459/460 | N313Q | + | + |
| 461/462 | T315V | + | + |
| 463/464 | A374G | + | + |
| 465/466 | E128S | + | + |
| 467/468 | V136A | + | + |
| 469/470 | E128R | + | |
| 471/472 | D370Q | + | |
| 473/474 | Q267V | + | + |
| 475/476 | R188M | + | + |
| 477/478 | R188F | + | ++ |
| 479/480 | G263S | + | ++ |
| 481/482 | R188S | + | + |
| 483/484 | Y339W | + | + |
| 485/486 | A100V/I251S | + | +++ |
| 487/488 | A131V | + | + |
| 489/490 | R188T | + | + |
| 491/492 | N141L | ++ | |
| 493/494 | Q134Y | + | |
| 495/496 | Q267M | + | + |
| 497/498 | G264N | + | + |
| 499/500 | Q134G | + | + |
| 501/502 | N185L | + | + |
| 503/504 | D370S | + | |
| 505/506 | Q267W | + | + |
| 507/508 | Q279M | + | ++ |
| 509/510 | Q267R | + | + |
| 511/512 | G264T | + | + |
| 513/514 | Q279L | + | ++ |
| 515/516 | G263R | + | |
| 517/518 | V136C | + | |
| 519/520 | N145E | ++ | + |
| 521/522 | R188G | + | + |
| 523/524 | T130A | + | + |
| 525/526 | G192N | + | + |
| 527/528 | R188L | + | + |
| 529/530 | S312I | ++ | |
| 531/532 | G129E | + | |
| 533/534 | T315E | + | ++ |
| 535/536 | N145A | + | + |
| 537/538 | Q267H | + | + |
| 539/540 | S372V | + | + |
| 541/542 | T130G | + | + |
| 543/544 | Q267T | | ++ |
| 545/546 | Q274W | + | + |
| 547/548 | V136M | + | + |
| 549/550 | S372C | + | + |
| 551/552 | N194T | + | + |
| 553/554 | Q375V | + | + |
| 555/556 | A135G | + | + |
| 557/558 | Q267I | + | + |
| 559/560 | N141L/Q220R | + | + |
| 561/562 | S324E | + | + |
| 563/564 | S160L | ++ | + |
| 565/566 | N141S | ++ | |
| 567/568 | S372L | + | ++ |
| 569/570 | A135Y | + | |
| 571/572 | N141V | + | + |
| 573/574 | N141A | + | + |
| 575/576 | A131R | + | + |
| 577/578 | A135E | + | + |
| 579/580 | S324Y | + | |
| 581/582 | T311D/R316K/D370G | + | ++ |
| 583/584 | I99V | + | + |
| 585/586 | A278N | + | + |
| 587/588 | V405Q | | + |
| 589/590 | T311D/S342N/D370G | ++ | ++ |
| 591/592 | N141Q/V198G | ++ | ++ |
| 593/594 | T311D/S342N | ++ | ++ |
| 595/596 | N141Q/T311D | ++ | ++ |
| 597/598 | Q279K/T311D/R377H/Q392Y | + | + |
| 599/600 | L186Y/V198G/T311D/ | ++ | +++ |
| | S342N/D370G/Q392Y | | |
| 601/602 | N141Q/Q392Y | ++ | + |
| 603/604 | T311D/D370G/Q392Y | ++ | ++ |
| 605/606 | N141Q/T311D/Q392Y | + | ++ |
| 607/608 | T311D/D370G | ++ | ++ |
| 609/610 | T311D/R316K/Q392Y | ++ | ++ |
| 611/612 | Y265G/T311D/Q392Y | + | +++ |
| 613/614 | N141Q/G192D | ++ | ++ |
| 615/616 | T311D | ++ | ++ |
| 617/618 | N141Q/Y265G/T311D/Q392Y | ++ | ++ |
| 619/620 | G192D/T311D/D370G/Q392Y | ++ | ++ |
| 621/622 | V198G/Y265G/T311D/R316K/D370G | + | +++ |
| 623/624 | N141Q/L186Y/Y265G/T311D | ++ | +++ |
| 625/626 | N141Q/V198G/Y265G/T311D/D370G | ++ | +++ |
| 627/628 | | + | + |

All activities were determined relative to the reference polypeptide of SEQ ID NO: 4. Levels of increased activity are defined as follows: “+” 0.9 to 1.2, “++” >1.2, “+++” >2. The reference sequence and all variants within this table are represented in the Sequence Listing as a fragment (i.e., residues 1-413) of their full length sequence, as described in Example 3.

### Example 4

Screening Results of Protease Variants Derived from SEQ ID NO: 628

The engineered protease variant used in this Example and represented by SEQ ID NO: 628 has amino acid residues 426-522 of SEQ ID NO: 2 deleted while retaining the His-tag sequence (see FIG. 1). This construct was used as the backbone for the construction of protease variants in this Example. Within the Sequence Listing, for purposes of consistency in description, SEQ ID NO: 628 and variants derived therefrom are represented as a fragment (residues 1-413) of their full-length sequence of the actual variants studied. The actual variants examined include residues 414-425 of SEQ ID NO: 2 and the His-tag sequence (see FIG. 1). Mutations identified in Tables 3.1 and 3.2 were recombined on this backbone and additional mutagenesis was performed. Variants were screened using the casein activity assay at pH 7.5 after a 1 hour pre-incubation at pH 4.5 in the presence of pepsin, as described in Example 2. Analysis of the data relative to SEQ ID NO: 628 is listed in Table 4.1. Some variants from these libraries were assayed in triplicate in the casein activity assay at pH 7.5 after no prior challenge, after a 1 hour pre-incubation at pH 4.5 in the presence of Pepsin, after a one hour pre-incubation at pH 4.45 in the presence of Pepsin, and after a one hour heat challenge at 64° C., as described in Example 2. Analysis of the average data relative to SEQ ID NO: 628 are listed in Table 4.2.

TABLE 4.1
Protease activity relative to SEQ ID NO: 628

| | Amino Acid | pH 4.5 |
|---|---|---|
| SEQ | Differences | Pepsin Challenge |
| ID NO: | (Relative to | Improvement Relative |
| (nt/aa) | SEQ ID NO: 628) | to SEQ ID NO: 628 |
| 629/630 | T242E | + |
| 631/632 | S157V | ++ |
| 633/634 | D250N | + |
| 635/636 | V373F | + |
| 637/638 | Q243R | + |
| 639/640 | Y336F | + |
| 641/642 | G187A | + |
| 643/644 | G240A | + |
| 645/646 | G280K | + |
| 647/648 | E271A | + |
| 649/650 | S237G | + |
| 651/652 | E386W | ++ |
| 653/654 | D382G | ++ |
| 655/656 | G280S | + |
| 657/658 | V373Y | + |
| 659/660 | V328M | + |
| 661/662 | S157R | + |
| 663/664 | S157A | + |
| 665/666 | G42W | + |
| 667/668 | Q243E | + |
| 669/670 | D382S | + |
| 671/672 | T391L | ++ |
| 673/674 | R381N | + |
| 675/676 | Q243M | + |
| 677/678 | T275V | + |
| 679/680 | S157I | ++ |
| 681/682 | V373S | + |
| 683/684 | S157T | + |
| 685/686 | G280D | + |
| 687/688 | A249G | + |
| 689/690 | Y239L | + |
| 691/692 | A384C | + |
| 693/694 | N139M | ++ |
| 695/696 | G240L | + |
| 697/698 | Q243T | ++ |
| 699/700 | D250L | + |
| 701/702 | D250A | + |
| 703/704 | D382T | ++ |
| 705/706 | I364A | + |
| 707/708 | S346V | + |
| 709/710 | V373M | + |
| 711/712 | S389P | ++ |
| 713/714 | V373C | + |
| 715/716 | D382R | + |
| 717/718 | V373E | + |
| 719/720 | D254E | + |
| 721/722 | L246I | + |
| 723/724 | D250F | + |
| 725/726 | G280T | + |
| 727/728 | V373A | ++ |
| 729/730 | N139K | + |
| 731/732 | T345I | + |
| 733/734 | V360S | + |
| 735/736 | T275A | + |
| 737/738 | A249M | + |
| 739/740 | I364V | + |
| 741/742 | S303V | + |
| 743/744 | A300R | + |
| 745/746 | Y239M | + |
| 747/748 | M269T | + |
| 749/750 | A135G/N141Q/S372L | ++ |
| 751/752 | T311D/T315V/S372L | ++ |
| 753/754 | V136M/N141V/T311D | ++ |
| 755/756 | N141V/R188M | ++ |
| 757/758 | A135E/V136M | + |
| 759/760 | V136M/N141Q/T311D | ++ |
| 761/762 | A135E/N141V/T315V | ++ |
| 763/764 | S372V | + |
| 765/766 | A135E/N141V/S160L/Q267I/S372V | ++ |
| 767/768 | A135G/V136M/N141V/S160L/N185E/ | ++ |
| | R188M/Q2671/T311D/T315V | |
| 769/770 | A135G/V136M | ++ |
| 771/772 | S160L/N185E | + |
| 773/774 | A135E/N141V/R188M/Q279M/T311D | ++ |
| 775/776 | A135G/V136M/N141Q | ++ |
| 777/778 | A135G/V136M/N141Q/S372L | ++ |
| 779/780 | A135G/N141V/S160L/N185E/ | ++ |
| | Q267I/Q279M | |
| 781/782 | A135G/N141V/S160L/Q267I | ++ |
| 783/784 | N141Q/R188M/T311D/S372V | ++ |
| 785/786 | S160L/N185E/R188M/Q279M/T311D | ++ |
| 787/788 | V136M/N141V/Q279M | ++ |
| 789/790 | A135G/V136M/N141V/S160L/ | ++ |
| | N185E/R188L | |
| 791/792 | N141Q/S372V | ++ |
| 793/794 | A135E/V136M/N141Q/T311D | ++ |
| 795/796 | N185E/T311D/T315V/S372V | ++ |
| 797/798 | A135G/N141V/R188M | ++ |
| 799/800 | V136M/N185E | ++ |
| 801/802 | A135E/N141Q | ++ |
| 803/804 | A135E/V136M/N141Q/Q279M/ | ++ |
| | T315V/S372L | |
| 805/806 | A135G/T311D/T315V | ++ |
| 807/808 | N141V | ++ |
| 809/810 | A135G/N141V | ++ |
| 811/812 | T311D/S372L | ++ |
| 813/814 | R188M/T311D | ++ |
| 815/816 | A135E/N141V/R188L/S372L | ++ |
| 817/818 | N141V/S160L/Q279M | ++ |
| 819/820 | N313Q/Q392Y | ++ |
| 821/822 | S342G/Q392Y | ++ |
| 823/824 | Q279L/Q392Y | + |
| 825/826 | E128T | + |
| 827/828 | V198G/S342G | ++ |
| 829/830 | N313Q | + |
| 831/832 | E128T/S312I | ++ |
| 833/834 | K50R | + |
| 835/836 | N145E/G263S | + |
| 837/838 | N313Q/S342G | + |
| 839/840 | Q279L/S312I | + |
| 841/842 | S312I/Q392Y | ++ |
| 843/844 | Q279K/S342G | + |
| 845/846 | Q279K/Q392Y | + |
| 847/848 | E128T/S342G | + |
| 849/850 | S342G | + |
| 851/852 | G263S | + |

All activities were determined relative to the reference polypeptide of SEQ ID NO: 628. Levels of increased activity are defined as follows: “+” 1 to 1.2, “++” >1.2. The reference sequence and all variants within this table are represented in the Sequence Listing as a fragment (residue 1-413) of their full length sequence, as described in Example 4

TABLE 4.2
Protease activity relative to SEQ ID NO: 628

| | | pH 4.5 and | pH 4.45 and | | |
|---|---|---|---|---|---|
| | | Pepsin | Pepsin | Unchallenged | Heat |
| | Amino Acid | Challenge | Challenge | Activity | Challenge |
| SEQ ID | Differences | Improvement | Improvement | Improvement | Improvement |
| NO: | (Relative to | Relative to | Relative to | Relative to | Relative to |
| (nt/aa) | SEQ ID NO: 628) | SEQ ID NO: 628 | SEQ ID NO: 628 | SEQ ID NO: 628 | SEQ ID NO: 628 |
| 853/854 | N139C | ++ | + | + | ++ |
| 855/856 | T345R | ++ | ++ | + | + |
| 857/858 | Q243L | + | + | + | ++ |
| 859/860 | H143A | + | ++ | | |
| 861/862 | A249S | + | + | + | ++ |
| 863/864 | G262S | ++ | ++ | + | ++ |
| 865/866 | N139R | + | + | + | + |
| 867/868 | M269Q | ++ | ++ | + | +++ |
| 869/870 | V328L | ++ | ++ | + | +++ |
| 871/872 | S157G | + | + | + | + |
| 873/874 | T156V | + | + | + | + |
| 875/876 | T242S | + | + | + | |
| 877/878 | N139L | ++ | ++ | + | ++ |
| 879/880 | G262A | ++ | ++ | + | ++ |
| 881/882 | T169S | ++ | ++ | + | + |
| 883/884 | S346T | ++ | ++ | + | ++ |
| 885/886 | H143N/S237A | ++ | ++ | + | ++ |
| 887/888 | V136M/S160L/N185E/ | ++ | ++ | + | +++ |
| | Q267I/T311D/S372L | | | | |
| 889/890 | A135E/S160L/T311D/ | ++ | +++ | + | ++++ |
| | S372L | | | | |
| 891/892 | A135G/N141V/T311D/ | ++ | ++ | + | ++++ |
| | T315V | | | | |
| 893/894 | N141V/T311D/T315V | ++ | ++ | ++ | ++++ |
| 895/896 | V136M/N141V/S160L/ | ++ | ++ | + | ++++ |
| | N185E/R188M/T311D/ | | | | |
| | T315V/S372V | | | | |
| 897/898 | A135G/N141V/T311D/ | ++ | +++ | + | ++++ |
| | T315V/S372L | | | | |
| 899/900 | A135G/N141Q/S160L/ | ++ | ++ | + | ++++ |
| | N185E/T311D/T315V | | | | |
| 901/902 | A135G/N141V/Q267I/ | ++ | ++ | + | ++++ |
| | T311D/T315V/S372V | | | | |
| 903/904 | A135G/V136M/N141V/ | ++ | ++ | + | ++++ |
| | S160L/T311D/T315V | | | | |
| 905/906 | A135G/V136M/N141V/ | ++ | ++ | + | +++ |
| | Q279M | | | | |
| 907/908 | A135G/N141Q/Q267I/ | ++ | ++ | + | ++++ |
| | Q279M/T311D/T315V | | | | |
| 909/910 | A135E/N141V/T311D/ | ++ | +++ | + | ++++ |
| | T315V/S372V | | | | |
| 911/912 | A135E/N141Q/S160L | ++ | ++ | + | +++ |
| 913/914 | A135G/N141Q/T311D/ | ++ | ++ | + | ++++ |
| | T315V | | | | |
| 915/916 | A135G/N141V/S160L/ | ++ | +++ | + | ++++ |
| | T311D/T315V/S372V | | | | |
| 917/918 | A135G/N141V/S160L/ | ++ | +++ | + | ++++ |
| | T311D/T315V | | | | |
| 919/920 | A135G/N141Q/Q267I/ | ++ | ++ | + | ++++ |
| | T311D/T315V/S372L | | | | |
| 921/922 | A135G/V136M/N141V/ | ++ | +++ | + | ++++ |
| | R188M/T311D | | | | |
| 923/924 | N141V/S160L/T311D | ++ | +++ | ++ | ++++ |
| 925/926 | A135E/N141V/S160L/ | ++ | ++ | + | ++++ |
| | Q279M/T311D/T315V/ | | | | |
| | S372L | | | | |
| 927/928 | N141V/S160L/N185E/ | ++ | ++ | + | ++++ |
| | Q279M/T311D/S372V | | | | |
| 929/930 | A135E/N141V/T311D/ | ++ | +++ | + | ++++ |
| | T315V | | | | |
| 931/932 | A135G/V136M/N141V/ | ++ | ++ | + | ++ |
| | S160L/T315V/S372V | | | | |
| 933/934 | A135E/N141V/S160L | ++ | ++ | + | +++ |
| 935/936 | A135E/V136M/S160L/ | ++ | ++ | ++ | ++++ |
| | Q279M/T311D/S372V | | | | |
| 937/938 | E128T/Q279K/S312I/ | ++ | ++ | + | +++ |
| | S342G | | | | |
| 939/940 | E128T/V198G/S312I/ | ++ | ++ | + | +++ |
| | S342G | | | | |
| 941/942 | G263S/S342G | + | ++ | + | |
| 943/944 | N145E/G263S/Q279L/ | ++ | ++ | + | ++ |
| | S312I/S342G/Q392Y | | | | |
| 945/946 | E128T/N145E/V198G/ | ++ | ++ | + | + |
| | S312I/N313Q/Q392Y | | | | |

All activities were determined relative to the reference polypeptide of SEQ ID NO: 628. Levels of increased activity are defined as follows: “+” 0.9 to 1.2, “++” >1.2, “+++” >2, “++++” >3. The reference sequence and all variants within this table are represented in the Sequence Listing as a fragment (residues 1-413) of their full length sequence, as described in Example 4

### Example 5

Screening Results of Protease Variants Derived from SEQ ID NO: 948

The engineered protease variant SEQ ID NO: 916 was truncated at the C-termini by 24 residues, including the removal of the 6×His tag, resulting in SEQ ID NO: 948. As such, SEQ ID NO: 948 has a carboxy terminus at amino acid residue 413. SEQ ID NO: 948 was used as the backbone for the construction of additional protease variants. Previously identified mutations were recombined on this backbone and additional mutagenesis was performed. Variants were screened using the casein activity assay at pH 7.5 after a 1 hour pre-incubation at pH 3.9 in the presence of pepsin as described in Example 2. Analysis of the data relative to SEQ ID NO: 948 is listed in Table 5.1. Some variants generated from these libraries were assayed in triplicate in the casein activity assay at pH 7.5 after no prior challenge, after a 1 hour pre-incubation at pH 3.8, 3.9, or 4.0 in the presence of pepsin, and after a 1 hour heat challenge at 71° C., as described in Example 2. Additionally, additional variants were assayed in triplicate in the casein activity assay at pH 6.5 unchallenged, as described in Example 2. Analysis of the average data relative to SEQ ID NO: 948 are listed in Table 5.2.

TABLE 5.1
Protease activity relative to SEQ ID NO: 948

| | | pH 3.9 pepsin |
|---|---|---|
| | Amino Acid | challenge |
| SEQ ID | Differences | improvement |
| NO: | (Relative to | relative to |
| (nt/aa) | SEQ ID NO: 948) | SEQ ID NO: 948 |
| 949/950 | S411V | ++ |
| 951/952 | S411R | ++ |
| 953/954 | A402G | ++ |
| 955/956 | A285S | + |
| 957/958 | I245V | ++ |
| 959/960 | N266H | ++ |
| 961/962 | P355A | ++ |
| 963/964 | M258W | ++ |
| 965/966 | A222G | + |
| 967/968 | Q140L | ++ |
| 969/970 | S268T | + |
| 971/972 | S411L | ++ |
| 973/974 | I225V | ++ |
| 975/976 | I245L | ++ |
| 977/978 | V283M | + |
| 979/980 | A406W | + |
| 981/982 | G410C | + |
| 983/984 | A406C | ++ |
| 985/986 | H143A/N145E/Q243L/S312I | +++ |
| 987/988 | N139L/H143A/N145E/S157G/S312I | +++ |
| 989/990 | N139C/S157G/T345R | +++ |
| 991/992 | N139C/M269Q | ++ |
| 993/994 | T156V/S157G/S342G/S346T | +++ |
| 995/996 | N139L/H143A | +++ |
| 997/998 | M269Q | ++ |
| 999/1000 | N139L/Q243L/V328L | +++ |
| 1001/1002 | M269Q/V328L | ++ |
| 1003/1004 | H143A/N145E/T169S | +++ |
| 1005/1006 | V328L | +++ |
| 1007/1008 | H143A/N145E/G262S | +++ |
| 1009/1010 | N145E/G262S/S312I/V328L | +++ |
| 1011/1012 | N139L/T156V/S157G | +++ |
| 1013/1014 | N139C/N145E/S312I | +++ |
| 1015/1016 | S312I | ++ |
| 1017/1018 | N139C | +++ |
| 1019/1020 | N139C/S312I | +++ |
| 1021/1022 | N139C/T156V | +++ |
| 1023/1024 | N139C/H143A/N145E/Q243L | +++ |
| 1025/1026 | N145E/S157G | +++ |
| 1027/1028 | N145E/S346T | +++ |
| 1029/1030 | N145E/G262A/S312I/V328L/ | +++ |
| | T345R/S346T | |
| 1031/1032 | N145E/G262A | ++ |
| 1033/1034 | S312I/S342G | ++ |
| 1035/1036 | H143A/Q243L | +++ |
| 1037/1038 | N139C/T345R | ++ |
| 1039/1040 | S342G | ++ |
| 1041/1042 | H143A/N145E/G262S/S342G | +++ |
| 1043/1044 | N139L/H143A/T169S | +++ |
| 1045/1046 | N139C/H143A/N145E/S312I | +++ |
| 1047/1048 | T169S | ++ |
| 1049/1050 | N139L/N145E/G262A/S312I/ | ++ |
| | V328L/S342G/T345R/S346T | |
| 1051/1052 | N139C/V328L | +++ |
| 1053/1054 | N139L/Q243L | ++ |
| 1055/1056 | N139L/H143A/V328L | +++ |
| 1057/1058 | N139L | ++ |
| 1059/1060 | N139C/H143A/Q243L | +++ |
| 1061/1062 | N139C/N145E | +++ |
| 1063/1064 | N145E/S312I | ++ |
| 1065/1066 | N145E/T169S | ++ |
| 1067/1068 | N139C/H143A/S157G/S312I | +++ |
| 1069/1070 | V84M/N139C/H143A | ++ |
| 1071/1072 | N145E/M269Q | ++ |
| 1073/1074 | H143A/N145E/S157G/M269Q/ | +++ |
| | S312I/V328L | |
| 1075/1076 | H143A/N145E/M269Q | +++ |
| 1077/1078 | S157G | ++ |
| 1079/1080 | N139L/H143A/S312I | +++ |

All activities were determined relative to the reference polypeptide of SEQ ID NO: 948. Levels of increased activity are defined as follows: “+” 1.0 to 1.2, “++” >1.2, “+++” >2

TABLE 5.2
Protease activity relative to SEQ ID NO: 948

| | | pH 3.9 and | pH 3.8 and | pH 4.0 and | pH 7.5 | pH 6.5 | |
|---|---|---|---|---|---|---|---|
| | | Pepsin | Pepsin | Pepsin | Unchallenged | Unchallenged | Heat |
| | | Challenge | Challenge | Challenge | Activity | Activity | Challenge |
| | | Improvement | Improvement | Improvement | Improvement | Improvement | Improvement |
| SEQ | | Relative to | Relative to | Relative to | Relative to | Relative to | Relative to |
| ID NO: | Amino Acid Differences | SEQ ID | SEQ ID | SEQ ID | SEQ ID | SEQ ID | SEQ ID |
| (nt/aa) | (Relative to SEQ ID NO: 948) | NO: 948 | NO: 948 | NO: 948 | NO: 948 | NO: 948 | NO: 948 |
| 1081/1082 | I256L | + | | | + | + | + |
| 1083/1084 | S411T | ++ | | + | + | + | + |
| 1085/1086 | V273F | + | | | | | |
| 1087/1088 | G409E | | | + | + | | + |
| 1089/1090 | S268G | + | | + | + | | + |
| 1091/1092 | D172Q | | | | + | + | |
| 1093/1094 | G409R | ++ | | + | + | + | + |
| 1095/1096 | H401L | + | | + | + | + | |
| 1097/1098 | T281C | + | | + | + | + | + |
| 1099/1100 | G410W | | | | + | | + |
| 1101/1102 | A253V | + | | + | + | + | + |
| 1103/1104 | A406M | | | + | + | + | + |
| 1105/1106 | V273T | ++ | | ++ | + | + | ++ |
| 1107/1108 | A406R | | | + | + | | + |
| 1109/1110 | I256M | + | | ++ | + | + | ++ |
| 1111/1112 | G410I | + | | ++ | + | + | + |
| 1113/1114 | V273M | + | | + | | | |
| 1115/1116 | V273L | ++ | | ++ | + | + | ++ |
| 1117/1118 | H143A/N145E/Q243L/V328L | +++ | ++++ | | + | + | + |
| 1119/1120 | N145E | ++ | +++ | | + | + | ++ |
| 1121/1122 | N139C/H143A/N145E/ | +++ | ++++ | | + | + | ++ |
| | V328L/S342G/T345R | | | | | | |
| 1123/1124 | N139L/N145E | ++ | +++ | | | | + |
| 1125/1126 | H143A/V328L/S342G/T345R | +++ | ++++ | | + | ++ | ++ |
| 1127/1128 | N145E/S342G/T345R | +++ | ++++ | | ++ | ++ | ++ |
| 1129/1130 | H143A | ++ | ++++ | | | | |
| 1131/1132 | N139C/H143A | ++ | ++++ | | | | + |
| 1133/1134 | N139C/N145E/V328L/ | +++ | ++++ | | + | ++ | ++ |
| | S342G/T345R | | | | | | |
| 1135/1136 | H143A/N145E/T169S/S312I/ | +++ | ++++ | | + | ++ | ++ |
| | V328L/T345R/S346T | | | | | | |
| 1137/1138 | H143A/Q243L/V328L/ | +++ | ++++ | | + | + | ++ |
| | S342G/T345R/S346T | | | | | | |
| 1139/1140 | N139C/H143A/S157G/ | +++ | ++++ | | + | + | ++ |
| | T169S/V328L/S346T | | | | | | |
| 1141/1142 | H143A/N145E/T156V/ | ++ | ++++ | | + | | + |
| | S312I/V328L | | | | | | |
| 1143/1144 | N139C/N145E/S157G/ | +++ | ++++ | | + | + | ++ |
| | S312I/V328L | | | | | | |
| 1145/1146 | H143A/V328L/S342G/ | +++ | ++++ | | + | ++ | ++ |
| | T345R/S346T | | | | | | |
| 1147/1148 | N139C/H143A/V328L | ++ | ++++ | | | | ++ |
| 1149/1150 | H143A/N145E | ++ | ++++ | | + | + | |
| 1151/1152 | H143A/N145E/S312I/ | +++ | ++++ | | + | + | |
| | S342G/T345R | | | | | | |
| 1153/1154 | H143A/N145E/V328L | +++ | ++++ | | + | | + |

All activities were determined relative to the reference polypeptide of SEQ ID NO: 948. Levels of increased activity are defined as follows:
“+” 0.9 to 1.2,
“++” > 1.2,
“+++” > 2,
“++++” > 3.5

### Example 6

Screening Results of Protease Variants Derived from SEQ ID NO: 1126

The engineered protease variant of SEQ ID NO: 1126 was selected as the backbone for the construction of additional protease variants. Previously identified mutations were recombined on this backbone and additional mutagenesis was performed. Variants were screened using the casein activity assay at pH 6.5 after a 1 hour pre-incubation at pH 3.35 in the presence of pepsin, as described in Example 2. Analysis of the data relative to SEQ ID NO: 1126 is listed in Table 6.1.

TABLE 6.1
Protease activity relative to SEQ ID NO: 1126

| | | pH 3.35 and |
|---|---|---|
| | Amino Acid | pepsin challenge |
| SEQ ID | Differences | improvement |
| NO: | (Relative to | relative to |
| (nt/aa) | SEQ ID NO: 1126) | SEQ ID NO: 1126 |
| 1155/1156 | Q279Y | + |
| 1157/1158 | D250T | ++ |
| 1159/1160 | A154C | + |
| 1161/1162 | S214Y | + |
| 1163/1164 | A249S | + |
| 1165/1166 | T275A | + |
| 1167/1168 | H137A | +++ |
| 1169/1170 | S161D | + |
| 1171/1172 | N180H | + |
| 1173/1174 | N174L | ++ |
| 1175/1176 | N139K | ++ |
| 1177/1178 | D254C | ++ |
| 1179/1180 | N145E | ++ |
| 1181/1182 | A278V | + |
| 1183/1184 | V136M | ++ |
| 1185/1186 | A154L/G413D | ++ |
| 1187/1188 | S294V | + |
| 1189/1190 | A154R | + |
| 1191/1192 | S237A | + |
| 1193/1194 | Q274G | + |
| 1195/1196 | H137N | +++ |
| 1197/1198 | G264P | + |
| 1199/1200 | Q274T | ++ |
| 1201/1202 | A278N | + |
| 1203/1204 | S214A | + |
| 1205/1206 | N185G | + |
| 1207/1208 | S214V | ++ |
| 1209/1210 | Q279T | + |
| 1211/1212 | V277G | + |
| 1213/1214 | G264I | + |
| 1215/1216 | S293A | ++ |
| 1217/1218 | S233L | + |
| 1219/1220 | A278Y | ++ |
| 1221/1222 | H173F | ++ |
| 1223/1224 | Q274L | + |
| 1225/1226 | S312C | +++ |
| 1227/1228 | Q279L | + |
| 1229/1230 | S302G | ++ |
| 1231/1232 | L238Q | + |
| 1233/1234 | S294W | ++ |
| 1235/1236 | G135A | ++ |
| 1237/1238 | S221E | + |
| 1239/1240 | D290S | + |
| 1241/1242 | A278S | ++ |
| 1243/1244 | G263Q | ++ |
| 1245/1246 | G263H | + |
| 1247/1248 | A278L | ++ |
| 1249/1250 | G263E | ++ |
| 1251/1252 | A154L | ++ |
| 1253/1254 | N139M | ++ |
| 1255/1256 | H137S | ++ |
| 1257/1258 | S233G | + |
| 1259/1260 | N139F | ++ |
| 1261/1262 | Q267I | + |
| 1263/1264 | S221L | + |
| 1265/1266 | H173S | + |
| 1267/1268 | S302P | + |
| 1269/1270 | S221V | + |
| 1271/1272 | Y239M | + |
| 1273/1274 | D290G | + |
| 1275/1276 | K163H | + |
| 1277/1278 | A292V | + |
| 1279/1280 | L246V | + |
| 1281/1282 | S214N | + |
| 1283/1284 | Q243S | + |
| 1285/1286 | S233I | ++ |
| 1287/1288 | S235Q | ++ |
| 1289/1290 | N145D | ++ |
| 1291/1292 | Q274V | ++ |
| 1293/1294 | Q279M | ++ |
| 1295/1296 | N185S | + |
| 1297/1298 | Q279K | ++ |
| 1299/1300 | N145W | +++ |
| 1301/1302 | D290E | + |
| 1303/1304 | S214P | ++ |
| 1305/1306 | T156V | ++ |
| 1307/1308 | T156C | ++ |
| 1309/1310 | T223S | + |
| 1311/1312 | A278V/G413D | ++ |
| 1313/1314 | D250C | + |
| 1315/1316 | Q267S | ++ |
| 1317/1318 | Y297F | + |
| 1319/1320 | S221Q | ++ |
| 1321/1322 | N194D | + |
| 1323/1324 | I251T | + |
| 1325/1326 | A253V/S411T | ++ |
| 1327/1328 | N145P/S157R/A253I/S268G/V273T/ | + |
| | T281V/S312Q/S346T/S411T | |
| 1329/1330 | N139C/S346A | ++ |
| 1331/1332 | A253V | + |
| 1333/1334 | S346T/S411T | ++ |
| 1335/1336 | A253V/S346T | ++ |
| 1337/1338 | N139C/S346T | ++ |
| 1339/1340 | S312Q/S346T | ++ |
| 1341/1342 | V273T/S312Q | ++ |
| 1343/1344 | A253I/T281V | ++ |
| 1345/1346 | S157R/A253V/V273T/S312Q/S346T/S411T | +++ |
| 1347/1348 | N139C/S157K/A253V/S268G/ | ++ |
| | V273F/T281V/S312Q/S346A | |
| 1349/1350 | A253V/V273T/S411T | ++ |
| 1351/1352 | N139C/A253I/S268F/V273T/T281V | +++ |
| 1353/1354 | N139C/S157R/S411T | +++ |
| 1355/1356 | S157G | ++ |
| 1357/1358 | V273T | ++ |
| 1359/1360 | N139C/A253V/S268G/V273F/ | +++ |
| | T281V/S312Q/S411T | |
| 1361/1362 | S157G/A253V/S411T | +++ |
| 1363/1364 | N139C/N145E/A253V/S346T | +++ |
| 1365/1366 | N139C/S157K/A253V/V273T/S312Q | +++ |
| 1367/1368 | N139C/S157G/S268G/V273T/S312Q/S346T | +++ |
| 1369/1370 | S157G/V273T/S312Q/S346T | +++ |
| 1371/1372 | N139C/S411T | +++ |
| 1373/1374 | N139C/A253V/S268G/V273F/ | +++ |
| | T281C/S312I/S346T/S411T | |
| 1375/1376 | S157K/V273F/S346T/S411T | +++ |
| 1377/1378 | N139C/N145E/S157G/V162I/ | ++ |
| | A253V/V273F/T281V/S312Q | |
| 1379/1380 | N139C/A253V/V273T/T281C/S312Q | +++ |
| 1381/1382 | S157G/A253V/S268F/V273F/T281V/S312Q | +++ |
| 1383/1384 | N139C/A253I/S268F | ++ |
| 1385/1386 | N139C/S157R/S312Q | ++ |
| 1387/1388 | A253V/V273T/T281C/S346T | +++ |
| 1389/1390 | S157K/A253V/S312I/S346T/S411T | +++ |
| 1391/1392 | S157R/V273T/S312Q/S346T/S411T | +++ |
| 1393/1394 | N139C/N145E/S157K/A253V/ | +++ |
| | S268G/T281C/S312Q | |
| 1395/1396 | N139C/V273T/S312Q/S346T | +++ |
| 1397/1398 | S157K/A253V/S268F/V273T/S312I/S346T | ++ |
| 1399/1400 | N139C/S268G/S346T | ++ |
| 1401/1402 | S157K | ++ |
| 1403/1404 | S268G/V273F/S312Q/S346T | +++ |
| 1405/1406 | N139C/S157K/A253V/S268F/V273F/S312Q | +++ |
| 1407/1408 | S157K/V273T/S312Q/S346T | +++ |
| 1409/1410 | N139C/S157G/A253V | +++ |
| 1411/1412 | N139C | ++ |
| 1413/1414 | N139C/A253V/T281V | +++ |
| 1415/1416 | N139C/S157G/A253V/S268G/V273T | +++ |
| 1417/1418 | A253V/S312Q/S411T | ++ |
| 1419/1420 | N139C/S268G/V273T | +++ |
| 1421/1422 | A253I | + |

All activities were determined relative to the reference polypeptide of SEQ ID NO: 1126. Levels of increased activity are defined as follows: “+” 0.9 to 1.2, “++” >1.2, “+++” >1.8

### Example 7

Screening Results of Protease Variants Derived from SEQ ID NO: 1368

The engineered protease variant of SEQ ID NO: 1368 was used as the backbone for the construction of additional protease variants. Previously identified mutations were recombined and additional mutagenesis was performed. Variants were screened using the casein activity assay at pH 6.5 after a 1 hour pre-incubation at pH 3.2 in the presence of pepsin, as described in Example 2. Select variants were additionally screened using the casein activity assay at pH 6.5 after a 1 hour pre-incubation at pH 3.14 in the presence of pepsin, as described in Example 2. Other select variants were additionally screened using the casein activity assay at pH 6.5 with no prior challenge, as described in Example 2. Analysis of the data relative to SEQ ID NO: 1368 is listed in Table 7.1.

TABLE 7.1
Protease activity relative to SEQ ID NO: 1368

| | | pH 3.2 and | pH 3.14 and | Unchallenged |
|---|---|---|---|---|
| | Amino Acid | pepsin challenge | pepsin challenge | activity |
| SEQ ID | Differences | improvement | improvement | improvement |
| NO: | (Relative to | relative to | relative to | relative to |
| (nt/aa) | SEQ ID NO: 1368) | SEQ ID NO: 1368 | SEQ ID NO: 1368 | SEQ ID NO: 1368 |
| 1423/1424 | D31G | + | | |
| 1425/1426 | S318R | + | | |
| 1427/1428 | S296R | + | | |
| 1429/1430 | S296M | + | | |
| 1431/1432 | D252P | + | | |
| 1433/1434 | S303A | + | | |
| 1435/1436 | A253C | + | | |
| 1437/1438 | G413C | + | | |
| 1439/1440 | E386P | + | | |
| 1441/1442 | G413A | + | | |
| 1443/1444 | Q312R | ++ | | |
| 1445/1446 | S235R | + | | |
| 1447/1448 | G412P | + | | |
| 1449/1450 | G342A | + | | |
| 1451/1452 | G413S | + | | |
| 1453/1454 | S302P | + | | |
| 1455/1456 | I371L | + | | |
| 1457/1458 | V405L | + | | |
| 1459/1460 | Q312A | + | | |
| 1461/1462 | S389P | + | | |
| 1463/1464 | S318P | + | | |
| 1465/1466 | T391S | + | | |
| 1467/1468 | G412T | + | | |
| 1469/1470 | A358S | + | | |
| 1471/1472 | S389C | + | | |
| 1473/1474 | S235V | ++ | | |
| 1475/1476 | T391L | + | | |
| 1477/1478 | C139N/T273V/D311T/L328V/ | | | ++ |
| | V372S | | | |
| 1479/1480 | D311T | | | + |
| 1481/1482 | Q312S | + | | + |
| 1483/1484 | V372S | + | | |
| 1485/1486 | C139N/A143H/G157S/L160S/ | | | ++ |
| | G268S/T273V/D311T/V315T | | | |
| 1487/1488 | A143H | | | + |
| 1489/1490 | C139N/A143H | | | ++ |
| 1491/1492 | C139N/L160S/Q312S/V372S | | | ++ |
| 1493/1494 | A143H/T273V/L328V | | | ++ |
| 1495/1496 | T346S | + | | + |
| 1497/1498 | G135A/C139N/L160S/G268S/ | | | ++ |
| | Q312S/G342S/T346S | | | |
| 1499/1500 | C139N/V141N/T273V | | | ++ |
| 1501/1502 | G135A/V141N/A143H/G268S/ | | | ++ |
| | T273V/Q312S/V372S | | | |
| 1503/1504 | C139N/V141N/A143H/D311T | | | ++ |
| 1505/1506 | C139N/G157S/G268S/L328V/ | | | ++ |
| | T346S/V372S | | | |
| 1507/1508 | T53A/C139N/V141N/A143H/ | | | ++ |
| | T273V/V372S | | | |
| 1509/1510 | C139N | | | ++ |
| 1511/1512 | C139N/V141N/A143H/T273V/ | | | ++ |
| | Q312S | | | |
| 1513/1514 | C139L | + | + | |
| 1515/1516 | H137A/C139N/S221Q/S233L/ | ++ | ++ | |
| | G413D | | | |
| 1517/1518 | S233L | + | + | |
| 1519/1520 | S221Q/Q279K | + | ++ | |
| 1521/1522 | H137N/C139N/S233L/Q279M | ++ | +++ | |
| 1523/1524 | S221Q | + | ++ | |
| 1525/1526 | C139L/S214V | + | ++ | |
| 1527/1528 | H137N/C139N/T156V | ++ | +++ | |
| 1529/1530 | C139L/S214V/S221Q | ++ | +++ | |
| 1531/1532 | S214V/S233L | + | ++ | |
| 1533/1534 | H137A/C139L/S221Q/S233L/ | + | +++ | |
| | Q279K | | | |
| 1535/1536 | H137A/C139L/S221Q | ++ | +++ | |
| 1537/1538 | H137N/C139L | ++ | +++ | |
| 1539/1540 | H137N/C139L/Q279K | ++ | +++ | |
| 1541/1542 | H137A/C139N | + | ++ | |
| 1543/1544 | H137N | ++ | +++ | |
| 1545/1546 | H137N/C139L/S221Q | ++ | +++ | |
| 1547/1548 | H137N/C139L/S214P/Q279M | ++ | +++ | |
| 1549/1550 | N266Y | + | + | |
| 1551/1552 | C139L/S221Q | + | ++ | |
| 1553/1554 | H137N/S221Q | ++ | +++ | |
| 1555/1556 | H137N/C139N | ++ | +++ | |
| 1557/1558 | H137N/T156V/S214V/Q312C | ++ | ++ | |
| 1559/1560 | H137N/S221Q/S233L | ++ | +++ | |
| 1561/1562 | H137N/C139N/T156V/S221Q | ++ | +++ | |
| 1563/1564 | H137A/C139L/S214V | ++ | +++ | |
| 1565/1566 | G413D | + | + | |
| 1567/1568 | Q279K | + | + | |
| 1569/1570 | Q279M | + | + | |
| 1571/1572 | H137A/C139L/S233L | ++ | ++ | |
| 1573/1574 | H137N/C139N/S214V/S233I | ++ | +++ | |
| 1575/1576 | S221Q/G413D | ++ | + | |
| 1577/1578 | H137N/S214V/S233L | ++ | +++ | |
| 1579/1580 | H137A/C139L/S214P | ++ | +++ | |
| 1581/1582 | H137N/C139N/S221Q/S233L/ | ++ | +++ | |
| | Q279M | | | |
| 1583/1584 | H137N/T156V | ++ | +++ | |
| 1585/1586 | H137N/C139L/S221Q/S233I | + | +++ | |
| 1587/1588 | S214V/S221Q | ++ | ++ | |
| 1589/1590 | H137N/S221Q/G413D | ++ | +++ | |
| 1591/1592 | H137A/C139L/S221Q/S233L/ | ++ | +++ | |
| | Q279M | | | |
| 1593/1594 | S214V | + | ++ | |
| 1595/1596 | H137A/C139L | ++ | +++ | |
| 1597/1598 | H137N/S233L | ++ | +++ | |
| 1599/1600 | H137A | + | +++ | |
| 1601/1602 | H137N/G413D | ++ | +++ | |
| 1603/1604 | H137N/S221Q/Q279K | ++ | +++ | |
| 1605/1606 | H137N/C139N/T156V/S214V/ | + | +++ | |
| | S233L/G413D | | | |
| 1607/1608 | H137N/C139L/T156V/S214V | + | +++ | |

All activities were determined relative to the reference polypeptide of SEQ ID NO: 1368. Levels of increased activity are defined as follows: “+” 0.9 to 1.2, “++” >1.2, “+++” >1.8

### Example 8

Screening Results of Protease Variants Derived from SEQ ID NO: 1548

The engineered protease variant of SEQ ID NO: 1548 was used as the backbone for the construction of additional protease variants. Previously identified mutations were recombined on this backbone. Variants were screened using the casein activity assay at pH 6.5 with no prior challenge and after a 1 hour pre-incubation at pH 2.8 in the presence of pepsin, as described in Example 2. Analysis of the data relative to SEQ ID NO: 1548 is listed in Table 8.1.

TABLE 8.1
Protease activity relative to SEQ ID NO: 1548

| | | Unchallenged | pH 2.8 and |
|---|---|---|---|
| | Amino Acid | activity | pepsin challenge |
| SEQ ID | Differences | improvement | improvement |
| NO: | (Relative to | relative to | relative to |
| (nt/aa) | SEQ ID NO: 1548) | SEQ ID NO: 1548 | SEQ ID NO: 1548 |
| 1609/1610 | N145E/T273L/V372S | ++ | ++ |
| 1611/1612 | N145E/1256M/T273L | ++ | + |
| 1613/1614 | S221Q/Q243L/T273L/L328V/V372S | ++ | ++ |
| 1615/1616 | V372S | + | + |
| 1617/1618 | N145E/S221Q/M279L/V372S/A406R | + | ++ |
| 1619/1620 | T273L/L328V | | ++ |
| 1621/1622 | N145E/T169S/T273L/T346S/A406R | ++ | |
| 1623/1624 | I256M/T273L | + | + |
| 1625/1626 | N145E/S221Q/T273L/L328V/T346S/ | + | ++ |
| | A406R | | |
| 1627/1628 | Q243L/T273L | | + |
| 1629/1630 | N145E/P214V/I256M/T273L/M279L/ | + | + |
| | L328V/V372S | | |
| 1631/1632 | T169S/S221Q/L328V/V372S/A406R | ++ | |
| 1633/1634 | V372S/A406R | ++ | + |
| 1635/1636 | T169S/L328V/V372S/A406R | ++ | |
| 1637/1638 | S221Q/V372S | ++ | + |
| 1639/1640 | N145E/S221Q/T273L/L328V/V372S | + | ++ |
| 1641/1642 | P214V/Q243L/T273L/L328V | + | + |
| 1643/1644 | N145E/S221Q | + | + |
| 1645/1646 | P214V/I256M/T273L/T346S/V372S | + | ++ |
| 1647/1648 | S221Q/A406R | + | ++ |
| 1649/1650 | T169S/V372S | ++ | |
| 1651/1652 | N145E/P214V/S221Q/T273L | | ++ |
| 1653/1654 | N145E/S221Q/T346S/V372S | + | ++ |
| 1655/1656 | Q243L/T273L/L328V/V372S/A406R | ++ | ++ |
| 1657/1658 | N145E/T169S/T273L/L328V/T346S | ++ | |
| 1659/1660 | L328V | + | + |
| 1661/1662 | T169S/T273L/V372S | ++ | |
| 1663/1664 | N145E/S221Q/L328V | ++ | ++ |
| 1665/1666 | T169S/P214V/T273L | ++ | |
| 1667/1668 | S221Q/T273L/L328V | + | ++ |
| 1669/1670 | S221Q | ++ | ++ |
| 1671/1672 | N145E/L328V | + | ++ |
| 1673/1674 | P214N/T346S | + | |
| 1675/1676 | Q312R | + | ++ |
| 1677/1678 | Y212S | + | |
| 1679/1680 | M279K | + | + |
| 1681/1682 | Y212S/Q312R | ++ | |
| 1683/1684 | N145E | + | + |
| 1685/1686 | A179S/T346S | + | |
| 1687/1688 | P214N | ++ | |
| 1689/1690 | T346S | + | + |
| 1691/1692 | V315R/V372Y | | + |
| 1693/1694 | V372Y | + | |
| 1695/1696 | Q375A | + | + |
| 1697/1698 | G264F | + | |
| 1699/1700 | A179S | + | |
| 1701/1702 | N185T | + | + |
| 1703/1704 | Q220R/V372Y | + | |
| 1705/1706 | S324D | + | |
| 1707/1708 | N145E/S221Q/T273L/L328V/V372S | + | + |
| 1709/1710 | N145E/S221Q/T273L/L328V/V372S | + | + |

All activities were determined relative to the reference polypeptide of SEQ ID NO: 1548. Levels of increased activity are defined as follows: “+” 0.9 to 1.2, “++” >1.2

SEQ ID NO: 1639/1640 was codon optimized for improved expression in E. coli, resulting in variations in the polynucleotide sequence and represented by SEQ ID NO: 1707 and SEQ ID NO: 1709.

### Example 9

Screening Results of Protease Variants Derived from SEQ ID NO: 4

SEQ ID NO: 4 was utilized as the backbone for the construction of novel variants. Variants were generated through saturation mutagenesis and by introducing truncations at the C terminus. Select variants were screened in triplicate using the casein activity assay at pH=7.5, as described in Example 2. Select variants were screened in triplicate using the casein activity assay at pH=7.5 after a one hour pre-incubation at pH=4.5 in the presence of pepsin, as described in Example 2. Other select variants were screened in triplicate using the casein activity assay at pH=7.5 with a one-hour heat challenge at 63° C., as described in Example 2. Analysis of the average data relative to SEQ ID NO: 4 are listed in Table 9.1.

TABLE 9.1
Protease activity relative to SEQ ID NO: 4

| | Amino Acid | Unchallenged | Gastric | Thermo |
|---|---|---|---|---|
| SEQ ID | Differences | FIOP | Challenge FIOP | challenge FIOP |
| NO: | (Relative to | Relative to | Relative to | Relative to |
| (nt/aa) | SEQ ID NO: 4) | SEQ ID NO: 4 | SEQ ID NO: 4 | SEQ ID NO: 4 |
| 1711/1712 | A135G | + | + | + |
| 1713/1714 | A135S | + | + | |
| 1715/1716 | A135V | + | + | + |
| 1717/1718 | A135L | + | | |
| 1719/1720 | A135R | + | + | |
| 1721/1722 | A135E | ++ | | + |
| 1723/1724 | A135P | + | | |
| 1725/1726 | A135H | + | ++ | |
| 1727/1728 | A135C | + | | |
| 1729/1730 | A135T | + | + | |
| 1731/1732 | A135Y | + | | |
| 1733/1734 | A135W | + | | |
| 1735/1736 | A135M | + | | |
| 1737/1738 | A135N | + | + | |
| 1739/1740 | H137S | + | | |
| 1741/1742 | H137A | + | | |
| 1743/1744 | H137N | + | ++ | |
| 1745/1746 | H137D | + | | |
| 1747/1748 | N139R | + | + | + |
| 1749/1750 | N139E | + | | |
| 1751/1752 | N139F | + | | |
| 1753/1754 | N139L | + | ++ | ++ |
| 1755/1756 | N139K | + | + | ++ |
| 1757/1758 | N139D | + | | |
| 1759/1760 | N139H | + | | |
| 1761/1762 | N139I | + | | |
| 1763/1764 | N139S | + | | |
| 1765/1766 | N141T | + | ++ | ++ |
| 1767/1768 | N141S | + | ++ | ++ |
| 1769/1770 | N141V | + | +++ | +++ |
| 1771/1772 | N141L | + | + | ++ |
| 1773/1774 | N141R | + | ++ | ++ |
| 1775/1776 | N141M | + | ++ | ++ |
| 1777/1778 | N141G | + | | |
| 1779/1780 | N141Y | + | ++ | ++ |
| 1781/1782 | N141I | + | ++ | ++ |
| 1783/1784 | N141C | + | +++ | ++ |
| 1785/1786 | N141F | + | ++ | ++ |
| 1787/1788 | N141A | + | +++ | +++ |
| 1789/1790 | N141D | + | | + |
| 1791/1792 | N141E | + | ++ | ++ |
| 1793/1794 | N141H | + | + | + |
| 1795/1796 | H143T | + | | |
| 1797/1798 | H143C | + | | |
| 1799/1800 | H143Q | ++ | +++ | |
| 1801/1802 | H143A | ++ | +++ | |
| 1803/1804 | H143D | | + | |
| 1805/1806 | H143S | + | +++ | |
| 1807/1808 | H143N | + | +++ | ++ |
| 1809/1810 | N145Q | + | ++ | + |
| 1811/1812 | N145T | + | | + |
| 1813/1814 | N145V | + | | |
| 1815/1816 | N145H | + | | |
| 1817/1818 | N145L | + | | |
| 1819/1820 | N145E | + | ++ | + |
| 1821/1822 | N145R | ++ | + | |
| 1823/1824 | N145D | + | | |
| 1825/1826 | N145R | + | + | |
| 1827/1828 | N145A | + | | ++ |
| 1829/1830 | N145F | + | | |
| 1831/1832 | N145S | + | | + |
| 1833/1834 | N145G | + | | + |
| 1835/1836 | N145I | + | | |
| 1837/1838 | N145K | ++ | | + |
| 1839/1840 | N145M | ++ | | |
| 1841/1842 | N145C | + | | + |
| 1843/1844 | N145W | + | | |
| 1845/1846 | S157A | + | + | + |
| 1847/1848 | S157E | + | | |
| 1849/1850 | S157P | + | + | + |
| 1851/1852 | S157V | + | +++ | ++ |
| 1853/1854 | S157T | + | ++ | ++ |
| 1855/1856 | S157N | + | | + |
| 1857/1858 | S157R | + | ++ | + |
| 1859/1860 | S157G | + | + | + |
| 1861/1862 | S157L | + | | + |
| 1863/1864 | S157W | + | | |
| 1865/1866 | S157K | + | + | + |
| 1867/1868 | S157C | + | | + |
| 1869/1870 | S157D | + | | |
| 1871/1872 | S157Q | + | | + |
| 1873/1874 | S157M | + | | + |
| 1875/1876 | S157H | + | | + |
| 1877/1878 | S157I | + | ++ | ++ |
| 1879/1880 | S157F | + | | |
| 1881/1882 | S160R | + | | + |
| 1883/1884 | S160V | + | + | ++ |
| 1885/1886 | S160C | + | ++ | + |
| 1887/1888 | S160Q | + | + | + |
| 1889/1890 | S160A | + | + | + |
| 1891/1892 | S160P | + | + | ++ |
| 1893/1894 | S160L | + | ++ | ++ |
| 1895/1896 | S160F | + | ++ | + |
| 1897/1898 | S160T | ++ | + | ++ |
| 1899/1900 | S160D | + | | + |
| 1901/1902 | S160Y | + | | |
| 1903/1904 | S160W | ++ | | |
| 1905/1906 | S160E | + | + | + |
| 1907/1908 | S160K | + | + | + |
| 1909/1910 | S160N | + | | + |
| 1911/1912 | S160M | + | ++ | ++ |
| 1913/1914 | S214G | + | | |
| 1915/1916 | S214M | ++ | +++ | ++ |
| 1917/1918 | S214L | + | ++ | ++ |
| 1919/1920 | S214Q | + | + | ++ |
| 1921/1922 | S214T | ++ | + | + |
| 1923/1924 | S214P | + | ++ | ++ |
| 1925/1926 | S214R | ++ | + | + |
| 1927/1928 | S214D | + | ++ | + |
| 1929/1930 | S214F | + | + | + |
| 1931/1932 | S214K | + | ++ | + |
| 1933/1934 | S214A | + | + | ++ |
| 1935/1936 | S214V | ++ | ++ | +++ |
| 1937/1938 | S214I | + | +++ | ++ |
| 1939/1940 | S214E | + | + | + |
| 1941/1942 | S214H | + | | + |
| 1943/1944 | S214Y | ++ | + | ++ |
| 1945/1946 | S214C | + | +++ | ++ |
| 1947/1948 | S214W | + | | + |
| 1949/1950 | S221L | ++ | ++ | ++ |
| 1951/1952 | S221T | + | + | ++ |
| 1953/1954 | S221I | ++ | ++ | +++ |
| 1955/1956 | S221R | + | | ++ |
| 1957/1958 | S221D | + | + | ++ |
| 1959/1960 | S221A | ++ | + | + |
| 1961/1962 | S221C | ++ | ++ | ++ |
| 1963/1964 | S221V | + | + | ++ |
| 1965/1966 | S221F | ++ | | + |
| 1967/1968 | S221G | + | | |
| 1969/1970 | S221P | + | | |
| 1971/1972 | S221K | ++ | + | ++ |
| 1973/1974 | S221Y | ++ | | ++ |
| 1975/1976 | S221E | + | ++ | ++ |
| 1977/1978 | S221Q | + | + | ++ |
| 1979/1980 | S221M | ++ | ++ | ++ |
| 1981/1982 | S221H | ++ | | ++ |
| 1983/1984 | S221W | + | | + |
| 1985/1986 | S268V | + | + | |
| 1987/1988 | S268Y | ++ | + | + |
| 1989/1990 | S268A | + | + | + |
| 1991/1992 | S268Q | + | + | ++ |
| 1993/1994 | S268P | + | + | + |
| 1995/1996 | S268G | + | + | + |
| 1997/1998 | S268T | + | | |
| 1999/2000 | S268H | + | | + |
| 2001/2002 | S268I | + | ++ | |
| 2003/2004 | S268F | + | | |
| 2005/2006 | S268N | + | | ++ |
| 2007/2008 | V273S | + | ++ | + |
| 2009/2010 | V273C | + | + | +++ |
| 2011/2012 | V273A | + | + | |
| 2013/2014 | V273L | + | + | ++ |
| 2015/2016 | V273F | | + | |
| 2017/2018 | V273T | ++ | + | +++ |
| 2019/2020 | V273M | + | + | |
| 2021/2022 | Q279R | + | + | + |
| 2023/2024 | Q279E | + | ++ | + |
| 2025/2026 | Q279F | ++ | + | + |
| 2027/2028 | Q279G | + | + | + |
| 2029/2030 | Q279T | + | ++ | + |
| 2031/2032 | Q279M | + | + | ++ |
| 2033/2034 | Q279L | + | + | ++ |
| 2035/2036 | Q279S | + | + | + |
| 2037/2038 | Q279A | + | + | |
| 2039/2040 | Q279K | + | ++ | + |
| 2041/2042 | Q279V | + | ++ | + |
| 2043/2044 | Q279W | + | | |
| 2045/2046 | Q279Y | ++ | ++ | ++ |
| 2047/2048 | Q279H | + | | + |
| 2049/2050 | T311S | + | | + |
| 2051/2052 | T311D | + | ++ | +++ |
| 2053/2054 | T311Q | + | | + |
| 2055/2056 | T311M | + | | |
| 2057/2058 | T311K | + | | |
| 2059/2060 | T311G | + | + | |
| 2061/2062 | T311A | ++ | | |
| 2063/2064 | T311E | + | | +++ |
| 2065/2066 | S312V | + | + | + |
| 2067/2068 | S312D | + | | |
| 2069/2070 | S312G | + | | |
| 2071/2072 | S312R | + | ++ | ++ |
| 2073/2074 | S312W | + | + | |
| 2075/2076 | S312M | ++ | ++ | + |
| 2077/2078 | S312L | + | | |
| 2079/2080 | S312N | + | | |
| 2081/2082 | S312E | + | ++ | ++ |
| 2083/2084 | S312A | + | + | ++ |
| 2085/2086 | S312T | + | | |
| 2087/2088 | S312Y | + | + | + |
| 2089/2090 | S312P | + | | |
| 2091/2092 | S312H | + | + | + |
| 2093/2094 | S312Q | + | ++ | ++ |
| 2095/2096 | S312K | + | ++ | +++ |
| 2097/2098 | S312C | + | + | + |
| 2099/2100 | S312I | + | ++ | ++ |
| 2101/2102 | T315E | + | | + |
| 2103/2104 | T315S | + | | |
| 2105/2106 | T315L | + | | |
| 2107/2108 | T315R | ++ | | |
| 2109/2110 | T315G | + | | |
| 2111/2112 | T315A | ++ | | |
| 2113/2114 | T315M | + | | |
| 2115/2116 | T315Y | + | | |
| 2117/2118 | T315K | + | | |
| 2119/2120 | T315Q | + | | + |
| 2121/2122 | T315D | + | | |
| 2123/2124 | T315W | + | | |
| 2125/2126 | T315C | ++ | | |
| 2127/2128 | T315V | + | + | + |
| 2129/2130 | T315I | ++ | | + |
| 2131/2132 | T315H | + | | |
| 2133/2134 | T315F | + | | |
| 2135/2136 | S342F | + | | |
| 2137/2138 | S342G | + | ++ | ++ |
| 2139/2140 | S342R | ++ | | ++ |
| 2141/2142 | S342Q | + | | + |
| 2143/2144 | S342E | + | | + |
| 2145/2146 | S342V | + | | |
| 2147/2148 | S342T | + | | |
| 2149/2150 | S342C | ++ | + | ++ |
| 2151/2152 | S342N | ++ | + | ++ |
| 2153/2154 | S342I | + | | |
| 2155/2156 | S342P | + | | |
| 2157/2158 | S342M | + | | + |
| 2159/2160 | S342A | + | ++ | ++ |
| 2161/2162 | S342W | + | | |
| 2163/2164 | S342K | + | ++ | ++ |
| 2165/2166 | S342D | + | | |
| 2167/2168 | S342Y | + | | |
| 2169/2170 | T345G | + | | |
| 2171/2172 | T345R | + | ++ | + |
| 2173/2174 | T345L | + | | |
| 2175/2176 | T345V | + | + | |
| 2177/2178 | T345A | + | + | + |
| 2179/2180 | T345M | + | | ++ |
| 2181/2182 | T345W | + | | |
| 2183/2184 | T345I | | + | |
| 2185/2186 | T345S | ++ | | |
| 2187/2188 | T345E | + | | |
| 2189/2190 | T345Y | + | | |
| 2191/2192 | T345D | + | | |
| 2193/2194 | T345Q | + | + | |
| 2195/2196 | T345F | + | | |
| 2197/2198 | T345C | | + | + |
| 2199/2200 | T345K | | ++ | ++ |
| 2201/2202 | S346T | + | ++ | ++ |
| 2203/2204 | S346Q | + | | |
| 2205/2206 | S346V | + | + | ++ |
| 2207/2208 | S346R | ++ | | |
| 2209/2210 | S346P | ++ | | |
| 2211/2212 | S346L | + | | + |
| 2213/2214 | S346D | + | | |
| 2215/2216 | S346W | + | + | ++ |
| 2217/2218 | S346G | + | | |
| 2219/2220 | S346A | ++ | | |
| 2221/2222 | S346C | + | + | + |
| 2223/2224 | S346M | + | | + |
| 2225/2226 | S346F | | + | ++ |
| 2227/2228 | S346N | + | | |
| 2229/2230 | S346Y | ++ | | ++ |
| 2231/2232 | S346K | + | | |
| 2233/2234 | A402* | + | | + |
| 2235/2236 | G409* | + | | + |
| 2237/2238 | G410* | + | + | + |
| 2239/2240 | G412* | + | + | + |
| 2241/2242 | G413* | + | + | ++ |

Levels of increased activity were determined relative to the reference polypeptide of SEQ ID NO: 1710 and are defined as follows: “+” >1, “++”> 1.1, “+++” >1.3

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

1. An engineered protease polypeptide, or a biologically active fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of SEQ ID NO: 4, 628, 948, 1126, 1368, or 1548, or to a reference sequence corresponding to SEQ ID NO: 4, 628, 948, 1126, 1368, or 1548, wherein the amino acid sequence comprises one or more substitutions relative to the reference sequence corresponding to residues 135-413 of SEQ ID NO: 4, 628, 948, 1126, 1368, or 1548, or relative to the reference sequence corresponding to SEQ ID NO: 4, 628, 948, 1126, 1368, or 1548.

2. (canceled)

3. (canceled)

4. The engineered protease polypeptide of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 6-1710, or to a reference sequence corresponding to an even-numbered SEQ ID NO. of SEQ ID NOs: 6-1710, wherein the amino acid sequence comprises one or more substitutions relative to the reference sequence corresponding to residues 135-413 of SEQ ID NO: 4 or 628, or relative to the reference sequence corresponding to SEQ ID NO: 4 or 628.

5. The engineered protease polypeptide of claim 1, wherein the amino acid sequence of the engineered protease polypeptide comprises at least a substitution at amino acid position 11, 31, 42, 45, 50, 53, 84, 99, 100, 126, 128, 129, 130, 131, 132, 134, 135, 136, 137, 138, 139, 140, 141, 143, 145, 151, 154, 156, 157, 159, 160, 161, 162, 163, 169, 172, 173, 174, 179, 180, 184, 185, 186, 187, 188, 190, 191, 192, 193, 194, 198, 199, 212, 214, 220, 221, 222, 223, 225, 231, 232, 233, 235, 237, 238, 239, 240, 242, 243, 245, 246, 249, 250, 251, 252, 253, 254, 256, 258, 262, 263, 264, 265, 266, 267, 268, 269, 271, 273, 274, 275, 277, 278, 279, 280, 281, 283, 285, 290, 292, 293, 294, 296, 297, 300, 302, 303, 311, 312, 313, 314, 315, 316, 318, 324, 328, 336, 339, 341, 342, 343, 345, 346, 355, 358, 360, 364, 367, 368, 369, 370, 371, 372, 373, 374, 375, 377, 381, 382, 384, 386, 389, 391, 392, 401, 402, 405, 406, 409, 410, 411, 412, or 413, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

6. The engineered protease polypeptide of claim 1, wherein the amino acid sequence of the engineered protease polypeptide comprises at least a substitution 11K, 31G, 42W, 45Y, 50R, 53A, 84M, 99V, 100V, 126T, 128G/I/K/L/P/R/S/T/V, 129E/F/H/I/K/L/R/S/T/V, 130A/F/G/N/V, 131E/P/R/T/V/Y, 132A/C/D/E/G/P/R/V/Y, 134A/C/D/E/G/I/L/M/N/P/S/T/V/W/Y, 135C/E/G/H/I/K/L/M/N/P/R/S/T/V/W/Y, 136C/G/I/M, 137A/D/N/S, 138Q, 139C/D/E/F/H/I/K/L/M/R/S, 140L, 141A/C/D/E/F/G/H/I/L/M/Q/R/S/T/V/W/Y, 143A/C/D/N/Q/S/T, 145A/C/D/E/F/G/H/I/K/L/P/Q/R/S/T/V/W, 151D/Q, 154C/D/L/R, 156C/V, 157A/C/D/E/F/G/H/I/K/L/M/N/P/Q/R/T/V/W, 159G, 160A/C/D/E/F/K/L/M/N/P/R/Q/T/V/W/Y, 161D/E/G/L/R, 1621, 163H/L, 169S, 172Q, 173F/S, 174L, 179K/S, 180H/L/M, 184A/D/G/L/M/Q/R, 185A/D/E/F/G/L/M/P/Q/R/S/T/V, 186A/R/S/T/Y, 187A, 188A/C/D/F/G/L/M/S/T/W, 190S, 191R, 192C/D/M/N, 193T, 194A/D/L/T, 198G, 199C/K/L, 212S, 214A/C/D/E/F/G/H/I/K/L/M/N/P/Q/R/T/V/W/Y, 220K/L/R, 221A/C/D/E/F/G/H/I/K/L/M/P/Q/R/T/V/W/Y, 222G, 223S, 225V, 231H/V, 232S, 233G/I/L, 235Q/R/V, 237A/G, 238Q, 239L/M, 240A/L, 242E/S, 243E/L/M/R/S/T, 245L/V, 246I/V, 249G/M/S, 250A/C/F/L/N/T, 251D/S, 252P, 253C/I/V, 254C/E, 256L/M, 258W, 262A/S, 263E/H/P/Q/R/S, 264A/C/F/I/L/N/P/R/T/V, 265C/G/R, 266H/T/Y, 267A/G/H/I/L/M/R/S/T/V/W, 268A/F/G/H/I/N/P/Q/T/V/Y, 269Q/T, 271A, 273A/C/F/L/M/S/T, 274A/G/K/L/T/V/W, 275A/V, 277D/G, 278L/N/S/V/Y, 279A/E/F/G/H/K/L/M/R/S/T/Y/V/W, 280D/K/S/T, 281C/V, 283M, 285S, 290E/G/S, 292V, 293A, 294V/W, 296M/R, 297F, 300R/V, 302G/P, 303A/V, 311A/E/D/G/K/M/Q/S, 312A/C/D/E/G/H/I/K/L/M/N/P/Q/R/T/V/W/Y, 313A/Q/S/T, 314G, 315A/C/D/E/F/G/H/I/K/L/M/Q/R/S/V/W/Y, 316K, 318N/P/R, 324A/D/E/I/R/V/W/Y, 328L/M, 336F, 339S/W, 341G, 342A/C/D/E/F/G/I/K/M/N/P/R/Q/T/V/W/Y, 343S, 345A/C/D/E/F/G/I/K/L/M/Q/R/S/V/W/Y, 346A/C/D/F/G/K/L/M/N/P/Q/R/T/V/W/Y, 355A, 358S, 360S, 364A/V, 367V, 368G/T, 369I/V/W, 370C/E/F/G/I/K/L/P/Q/R/S/V, 371L, 372A/C/F/L/R/V/Y, 373A/C/E/F/M/S/Y, 374E/G/L/R/S/W/Y, 375A/E/I/L/M/S/T/V, 377H, 381N, 382G/R/S/T, 384C, 386P/W, 389C/P, 391L/S, 392Y, 401L, 402G/*, 405L/Q, 406C/M/R/W, 409E/R/*, 410C/I/W/*, 411L/R/T/V, 412P/T/*, or 413A/C/D/S/*, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

7-9. (canceled)

10. The engineered protease polypeptide of claim 1, wherein the amino acid sequence of the engineered protease polypeptide comprises at least a substitution set at amino acid positions 135/141/160/311/315/372, 143/328/342/345, 139/157/268/273/312/346, or 137/139/214/279, wherein the amino acid positions are relative to the reference sequence corresponding to residues 135-413 of SEQ ID NO: 4 or 628, or relative to the reference sequence corresponding to SEQ ID NO: 4 or 628.

11. (canceled)

12. (canceled)

13. The engineered protease polypeptide of claim 1, wherein the amino acid sequence of the engineered protease polypeptide comprises at least a substitution or substitution set at amino acid position(s) 185, 134, 129, 135, 184, 132, 186, 193, 263, 370, 45/134, 199, 368, 161, 141, 267, 179, 264, 160, 138, 131, 372, 151, 274, 128, 339, 313, 374, 314, 191, 324, 315, 375, 136, 220, 194, 231, 277, 369, 251, 180, 163, 343, 264/279, 279, 232, 141/300, 367, 266, 188, 130, 318, 265, 341, 190, 145, 126/192, 11/220, 192, 370/392, 99/278, 265/311, 84/159/265/279/311/370, 311/316, 342/370, 265/311/370, 192/311/316, 141/154/192, 265/311/316/342, 279/311/316, 141/265/279/311/342, 141/192/311/316/370, 141/265/311, 198/279, 392, 342/370/392, 141/198/265, 265/392, 184/267, 342, 312, 100/251, 141/220, 311/316/370, 99, 278, 405, 311/342/370, 141/198, 311/342, 141/311, 279/311/377/392, 186/198/311/342/370/392, 141/392, 311/370/392, 141/311/392, 311/370, 311/316/392, 265/311/392, 141/192, 311, 141/265/311/392, 192/311/370/392, 198/265/311/316/370, 141/186/265/311, or 141/198/265/311/370, wherein the amino acid positions are relative to the reference sequence corresponding to residues 135-413 of SEQ ID NO: 4 or 628, or relative to the reference sequence corresponding to SEQ ID NO: 4 or 628.

14. (canceled)

15. (canceled)

16. The engineered protease polypeptide of claim 1, wherein the amino acid sequence of the engineered protease polypeptide comprises at least a substitution at amino acid position 135, 137, 139, 141, 143, 145, 145, 157, 160, 214, 221, 268, 273, 279, 311, 312, 315, 315, 342, 345, 346, 402, 409, 410, 412, or 413, or combinations thereof, wherein the amino acid positions are relative to the reference sequence corresponding to residues 135-413 of SEQ ID NO: 4 or 628, or relative to the reference sequence corresponding to SEQ ID NO: 4 or 628.

17. (canceled)

18. The engineered protease polypeptide of claim 1, wherein the amino acid sequence of the engineered protease polypeptide comprises at least a substitution or substitution set at amino acid position(s) 242, 157, 250, 373, 243, 336, 187, 240, 280, 271, 237, 386, 382, 328, 42, 391, 381, 275, 249, 239, 384, 139, 364, 346, 389, 254, 246, 345, 360, 303, 300, 269, 135/141/372, 311/315/372, 136/141/311, 141/188, 135/136, 135/141/315, 372, 135/141/160/267/372, 135/136/141/160/185/188/267/311/315, 160/185, 135/141/188/279/311, 135/136/141, 135/136/141/372, 135/141/160/185/267/279, 135/141/160/267, 141/188/311/372, 160/185/188/279/311, 136/141/279, 135/136/141/160/185/188, 141/372, 135/136/141/311, 185/311/315/372, 135/141/188, 136/185, 135/141, 135/136/141/279/315/372, 135/311/315, 141, 311/372, 188/311, 135/141/188/372, 141/160/279, 313/392, 342/392, 279/392, 128, 198/342, 313, 128/312, 50, 145/263, 313/342, 279/312, 312/392, 279/342, 128/342, 342, 263, 143, 262, 156, 169, 143/237, 136/160/185/267/311/372, 135/160/311/372, 135/141/311/315, 141/311/315, 136/141/160/185/188/311/315/372, 135/141/311/315/372, 135/141/160/185/311/315, 135/141/267/311/315/372, 135/136/141/160/311/315, 135/136/141/279, 135/141/267/279/311/315, 135/141/160, 135/141/160/311/315/372, 135/141/160/311/315, 135/136/141/188/311, 141/160/311, 135/141/160/279/311/315/372, 141/160/185/279/311/372, 135/136/141/160/315/372, 135/136/160/279/311/372, 128/279/312/342, 128/198/312/342, 263/342, 145/263/279/312/342/392, or 128/145/198/312/313/392, wherein the amino acid positions are relative to the reference sequence corresponding to residues 135-413 of SEQ ID NO: 4 or 628, or relative to the reference sequence corresponding to SEQ ID NO: 4 or 628.

19-27. (canceled)

28. The engineered protease polypeptide of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 6-1710, or to a reference sequence corresponding to an even-numbered SEQ ID NO. of SEQ ID NOs: 6-1710, wherein the amino acid sequence comprises one or more substitutions relative to the reference sequence corresponding to residues 135-413 of SEQ ID NO: 948, 1126, 1368, or 1548, or relative to the reference sequence corresponding to SEQ ID NO: 948, 1126, 1368, or 1548.

29. The engineered protease polypeptide of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 948, 1126, 1368, or 1548, or to the reference sequence corresponding to SEQ ID NO: 948, 1126, 1368, or 1548, wherein the amino acid sequence comprises one or more substitutions relative to the reference sequence corresponding to residues 135-413 of SEQ ID NO: 948, 1126, 1368, or 1548, or relative to the reference sequence corresponding to SEQ ID NO: 948, 1126, 1368, or 1548.

30. The engineered protease polypeptide of claim 28, wherein the amino acid sequence of the engineered protease polypeptide comprises at least a substitution at amino acid position 11, 31, 42, 45, 50, 53, 84, 99, 100, 126, 128, 129, 130, 131, 132, 134, 135, 136, 137, 138, 139, 140, 141, 143, 145, 151, 154, 156, 157, 159, 160, 161, 162, 163, 169, 172, 173, 174, 179, 180, 184, 185, 186, 187, 188, 190, 191, 192, 193, 194, 198, 199, 212, 214, 220, 221, 222, 223, 225, 231, 232, 233, 235, 237, 238, 239, 240, 242, 243, 245, 246, 249, 250, 251, 252, 253, 254, 256, 258, 262, 263, 264, 265, 266, 267, 268, 269, 271, 273, 274, 275, 277, 278, 279, 280, 281, 283, 285, 290, 292, 293, 294, 296, 297, 300, 302, 303, 311, 312, 313, 314, 315, 316, 318, 324, 328, 336, 339, 341, 342, 343, 345, 346, 355, 358, 360, 364, 367, 368, 369, 370, 371, 372, 373, 374, 375, 377, 381, 382, 384, 386, 389, 391, 392, 401, 402, 405, 406, 409, 410, 411, 412, or 413, or combinations thereof, wherein the amino acid positions are relative to the reference sequence corresponding to residues 135-413 of SEQ ID NO: 948, 1126, 1368, or 1548, or to the reference sequence corresponding to SEQ ID NO: 948, 1126, 1368, or 1548.

31. The engineered protease polypeptide of claim 28, wherein the amino acid sequence of the engineered protease polypeptide comprises at least a substitution or amino acid residue 11K, 31G, 42W, 45Y, 50R, 53A, 84M, 99V, 100V, 126T, 128G/I/K/L/P/R/S/T/V, 129E/F/H/I/K/L/R/S/T/V, 130A/F/G/N/V, 131E/P/R/T/V/Y, 132A/C/D/E/G/P/R/V/Y, 134A/C/D/E/G/I/L/M/N/P/S/T/V/W/Y, 135A/C/E/G/H/I/K/L/M/N/P/R/S/T/V/W/Y, 136C/G/I/M, 137A/D/N/S, 138Q, 139C/D/E/F/H/I/K/L/M/N/R/S, 140L, 141A/C/D/E/F/G/H/I/L/M/N/Q/R/S/T/V/W/Y, 143A/C/D/H/N/Q/S/T, 145A/C/D/E/F/G/H/I/K/L/P/Q/R/S/T/V/W, 151D/Q, 154C/D/L/R, 156C/V, 157A/C/D/E/F/G/H/I/K/L/M/N/P/Q/R/S/T/V/W, 159G, 160A/C/D/E/F/K/L/M/N/P/R/Q/S/T/V/W/Y, 161D/E/G/L/R, 162I, 163H/L, 169S, 172Q, 173F/S, 174L, 179K/S, 180H/L/M, 184A/D/G/L/M/Q/R, 185A/D/E/F/G/L/M/P/Q/R/S/T/V, 186A/R/S/T/Y, 187A, 188A/C/D/F/G/L/M/S/T/W, 190S, 191R, 192C/D/M/N, 193T, 194A/D/L/T, 198G, 199C/K/L, 212S, 214A/C/D/E/F/G/H/I/K/L/M/N/P/Q/R/T/V/W/Y, 220K/L/R, 221A/C/D/E/F/G/H/I/K/L/M/P/Q/R/T/V/W/Y, 222G, 223S, 225V, 231H/V, 232S, 233G/I/L, 235Q/R/V, 237A/G, 238Q, 239L/M, 240A/L, 242E/S, 243E/L/M/R/S/T, 245L/V, 246I/V, 249G/M/S, 250A/C/F/L/N/T, 251D/S/T, 252P, 253C/I/V, 254C/E, 256L/M, 258W, 262A/S, 263E/H/P/Q/R/S, 264A/C/F/I/L/N/P/R/T/V, 265C/G/R, 266H/T/Y, 267A/G/H/I/L/M/R/S/T/V/W, 268A/F/G/H/I/N/P/Q/S/T/V/Y, 269Q/T, 271A, 273A/C/F/L/M/S/T/V, 274A/G/K/L/T/V/W, 275A/V, 277D/G, 278L/N/S/V/Y, 279A/E/F/G/H/K/L/M/R/S/T/Y/V/W, 280D/K/S/T, 281C/V, 283M, 285S, 290E/G/S, 292V, 293A, 294V/W, 296M/R, 297F, 300R/V, 302G/P, 303A/V, 311A/E/D/G/K/M/Q/S/T, 312A/C/D/E/G/H/I/K/L/M/N/P/Q/R/S/T/V/W/Y, 313A/Q/S/T, 314G, 315A/C/D/E/F/G/H/I/K/L/M/Q/R/S/T/V/W/Y, 316K, 318N/P/R, 324A/D/E/I/R/V/W/Y, 328L/M/V, 336F, 339S/W, 341G, 342A/C/D/E/F/G/I/K/M/N/P/R/Q/S/T/V/W/Y, 343S, 345A/C/D/E/F/G/I/K/L/M/Q/R/S/V/W/Y, 346A/C/D/F/G/K/L/M/N/P/Q/R/S/T/V/W/Y, 355A, 358S, 360S, 364A/V, 367V, 368G/T, 369I/V/W, 370C/E/F/G/I/K/L/P/Q/R/S/V, 371L, 372A/C/F/L/R/S/V/Y, 373A/C/E/F/M/S/Y, 374E/G/L/R/S/W/Y, 375A/E/I/L/M/S/T/V, 377H, 381N, 382G/R/S/T, 384C, 386P/W, 389C/P, 391L/S, 392Y, 401L, 402G/*, 405L/Q, 406C/M/R/W, 409E/R/*, 410C/I/W/*, 411L/R/T/V, 412P/T/*, or 413A/C/D/S/*, or combinations thereof, wherein the amino acid positions are relative to the reference sequence corresponding to residues 135-413 of SEQ ID NO: 948, 1126, 1368, or 1548, or to the reference sequence corresponding to SEQ ID NO: 948, 1126, 1368, or 1548.

32-34. (canceled)

35. The engineered protease polypeptide of claim 28, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 948, or to the reference sequence corresponding to SEQ ID NO: 948, wherein the amino acid sequence comprises one or more substitutions relative to the reference sequence corresponding to residues 135-413 of SEQ ID NO: 948, or relative to the reference sequence corresponding to SEQ ID NO: 948.

36. The engineered protease polypeptide of claim 28, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 950-1154, or to a reference sequence corresponding to an even-numbered SEQ ID NO. of SEQ ID NOs: 950-1154, wherein the amino acid sequence comprises one or more substitutions relative to the reference sequence corresponding to residues 135-413 of SEQ ID NO: 948, or relative to the reference sequence corresponding to SEQ ID NO: 948.

37. The engineered protease polypeptide of claim 35, wherein the amino acid sequence of the engineered protease polypeptide comprises at least a substitution or substitution set at amino acid position(s) 411, 402, 285, 245, 266, 355, 258, 222, 140, 268, 225, 283, 406, 410, 143/145/243/312, 139/143/145/157/312, 139/157/345, 139/269, 156/157/342/346, 139/143, 269, 139/243/328, 269/328, 143/145/169, 328, 143/145/262, 145/262/312/328, 139/156/157, 139/145/312, 312, 139, 139/312, 139/156, 139/143/145/243, 145/157, 145/346, 145/262/312/328/345/346, 145/262, 312/342, 143/243, 139/345, 342, 143/145/262/342, 139/143/169, 139/143/145/312, 169, 139/145/262/312/328/342/345/346, 139/328, 139/243, 139/143/328, 139/143/243, 139/145, 145/312, 145/169, 139/143/157/312, 84/139/143, 145/269, 143/145/157/269/312/328, 143/145/269, 157, 139/143/312, 256, 273, 409, 172, 401, 281, 253, 143/145/243/328, 145, 139/143/145/328/342/345, 143/328/342/345, 145/342/345, 143, 139/145/328/342/345, 143/145/169/312/328/345/346, 143/243/328/342/345/346, 139/143/157/169/328/346, 143/145/156/312/328, 139/145/157/312/328, 143/328/342/345/346, 143/145, 143/145/312/342/345, or 143/145/328, wherein the amino acid positions are relative to the reference sequence corresponding to residues 135-413 of SEQ ID NO: 948, or to the reference sequence corresponding to SEQ ID NO: 948.

38. (canceled)

39. (canceled)

40. The engineered protease polypeptide of claim 28, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 1126, or to the reference sequence corresponding to SEQ ID NO: 1126, wherein the amino acid sequence comprises one or more substitutions relative to the reference sequence corresponding to residues 135-413 of SEQ ID NO: 1126, or relative to the reference sequence corresponding to SEQ ID NO: 1126.

41. The engineered protease polypeptide of claim 28, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 1156-1422, or to a reference sequence corresponding to an even-numbered SEQ ID NO. of SEQ ID NOs: 1156-1422, wherein the amino acid sequence comprises one or more substitutions relative to the reference sequence corresponding to residues 135-413 of SEQ ID NO: 1126, or relative to the reference sequence corresponding to SEQ ID NO: 1126.

42. The engineered protease polypeptide of claim 40, wherein the amino acid sequence of the engineered protease polypeptide comprises at least a substitution or substitution set at amino acid position(s) 279, 250, 154, 214, 249, 275, 137, 161, 180, 174, 139, 254, 145, 278, 136, 154/413, 294, 237, 274, 264, 185, 277, 293, 233, 173, 312, 302, 238, 135, 221, 290, 263, 267, 239, 163, 292, 246, 243, 235, 156, 223, 278/413, 297, 194, 251, 253/411, 145/157/253/268/273/281/312/346/411, 139/346, 253, 346/411, 253/346, 312/346, 273/312, 253/281, 157/253/273/312/346/411, 139/157/253/268/273/281/312/346, 253/273/411, 139/253/268/273/281, 139/157/411, 157, 273, 139/253/268/273/281/312/411, 157/253/411, 139/145/253/346, 139/157/253/273/312, 139/157/268/273/312/346, 157/273/312/346, 139/411, 139/253/268/273/281/312/346/411, 157/273/346/411, 139/145/157/162/253/273/281/312, 139/253/273/281/312, 157/253/268/273/281/312, 139/253/268, 139/157/312, 253/273/281/346, 157/253/312/346/411, 157/273/312/346/411, 139/145/157/253/268/281/312, 139/273/312/346, 157/253/268/273/312/346, 139/268/346, 268/273/312/346, 139/157/253/268/273/312, 139/157/253, 139/253/281, 139/157/253/268/273, 253/312/411, or 139/268/273, wherein the amino acid positions are relative to the reference sequence corresponding to residues 135-413 of SEQ ID NO: 1126, or to the reference sequence corresponding to SEQ ID NO: 1126.

43. (canceled)

44. The engineered protease polypeptide of claim 28, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 1368, or to the reference sequence corresponding to SEQ ID NO: 1368, wherein the amino acid sequence comprises one or more substitutions relative to the reference sequence corresponding to residues 135-413 of SEQ ID NO: 1368, or relative to the reference sequence corresponding to SEQ ID NO: 1368.

45. The engineered protease polypeptide of claim 28, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 1424-1608, or to a reference sequence corresponding to an even-numbered SEQ ID NO. of SEQ ID NOs: 1424-1608, wherein the amino acid sequence comprises one or more substitutions relative to the reference sequence corresponding to residues 135-413 of SEQ ID NO: 1368, or relative to the reference sequence corresponding to SEQ ID NO: 1368.

46. The engineered protease polypeptide of claim 44, wherein the amino acid sequence of the engineered protease polypeptide comprises at least a substitution or substitution set at amino acid position(s) 31, 318, 296, 252, 303, 253, 413, 386, 312, 235, 412, 342, 302, 371, 405, 389, 391, 358, 139/273/311/328/372, 311, 372, 139/143/157/160/268/273/311/315, 143, 139/143, 139/160/312/372, 143/273/328, 346, 135/139/160/268/312/342/346, 139/141/273, 135/141/143/268/273/312/372, 139/141/143/311, 139/157/268/328/346/372, 53/139/141/143/273/372, 139, 139/141/143/273/312, 137/139/221/233/413, 233, 221/279, 137/139/233/279, 221, 139/214, 137/139/156, 139/214/221, 214/233, 137/139/221/233/279, 137/139/221, 137/139, 137/139/279, 137, 137/139/214/279, 266, 139/221, 137/221, 137/156/214/312, 137/221/233, 137/139/156/221, 137/139/214, 279, 137/139/233, 137/139/214/233, 221/413, 137/214/233, 137/156, 137/139/221/233, 214/221, 137/221/413, 214, 137/233, 137/413, 137/221/279, 137/139/156/214/233/413, or 137/139/156/214, wherein the amino acid positions are relative to the reference sequence corresponding to residues 135-413 of SEQ ID NO: 1368, or relative to the reference sequence corresponding to SEQ ID NO: 1368.

47. (canceled)

48. The engineered protease polypeptide of claim 28, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to the reference sequence corresponding to residues 135-413 of SEQ ID NO: 1548, or to the reference sequence corresponding to SEQ ID NO: 1548, wherein the amino acid sequence comprises one or more substitutions relative to the reference sequence corresponding to residues 135-413 of SEQ ID NO: 1548, or relative to the reference sequence corresponding to SEQ ID NO: 1548.

49. The engineered protease polypeptide of claim 28, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 135-413 of an even-numbered SEQ ID NO. of SEQ ID NOs: 1610-1710, or to a reference sequence corresponding to an even-numbered SEQ ID NO. of SEQ ID NOs: 1610-1710, wherein the amino acid sequence comprises one or more substitutions relative to the reference sequence corresponding to residues 135-413 of SEQ ID NO: 1548, or relative to the reference sequence corresponding to SEQ ID NO: 1548.

50. The engineered protease polypeptide of claim 48, wherein the amino acid sequence of the engineered protease polypeptide comprises at least a substitution or substitution set at amino acid position(s) 145/273/372, 145/256/273, 221/243/273/328/372, 372, 145/221/279/372/406, 273/328, 145/169/273/346/406, 256/273, 145/221/273/328/346/406, 243/273, 145/214/256/273/279/328/372, 169/221/328/372/406, 372/406, 169/328/372/406, 221/372, 145/221/273/328/372, 214/243/273/328, 145/221, 214/256/273/346/372, 221/406, 169/372, 145/214/221/273, 145/221/346/372, 243/273/328/372/406, 145/169/273/328/346, 328, 169/273/372, 145/221/328, 169/214/273, 221/273/328, 221, 145/328, 214/346, 312, 212, 279, 212/312, 145, 179/346, 214, 346, 315/372, 375, 264, 179, 185, 220/372, or 324, wherein the amino acid positions are relative to the reference sequence corresponding to residues 135-413 of SEQ ID NO: 1548, or relative to the reference sequence corresponding to SEQ ID NO: 1548.

51-56. (canceled)

57. The engineered protease polypeptide of claim 1, comprising an amino acid sequence comprising residues 135-413 of an even-numbered SEQ ID NO. of SEQ ID NOs: 6-1710, or an amino acid sequence comprising an even-numbered SEQ ID NO. of SEQ ID NOs: 6-1710, wherein optionally the amino acid sequence comprises 1, 2, 3, 4, 5, 6, 7, 8, 9 or up to 10 substitutions.

58. (canceled)

59. The engineered protease polypeptide of claim 1, wherein the amino acid sequence of the engineered protease polypeptide comprises amino acid residues 135-413 or amino acid residues 128-413, wherein the engineered protease polypeptide is proteolytically active or is an active protease.

60-64. (canceled)

65. The engineered protease polypeptide of claim 59, wherein the proteolytic active polypeptide or active protease is characterized by an improved property selected from:
i) increased protease activity, ii) increased resistance to pepsin, iii) increased stability and/or activity at acidic pH, iv) increased stability and/or activity at neutral pH, or v) increased thermostability, or any combination of i), ii), iii), iv), and v) as compared to a reference protease, wherein the reference protease has an amino acid sequence corresponding to residues 135-413 of SEQ ID NO: 4, 628, 948, 1126, 1368, or 1548, or an amino acid sequence corresponding to SEQ ID NO: 4, 628, 948, 1126, 1368, or 1548.

66. (canceled)

67. (canceled)

68. The engineered protease polypeptide of claim 1, further comprising a Big1 domain at or fused to the carboxy terminus of the engineered protease polypeptide.

69-71. (canceled)

72. The engineered protease polypeptide of claim 1, further comprising a signal sequence.

73. (canceled)

74. An engineered protease polypeptide comprising at least a carboxy terminal deletion of SEQ ID NO: 2, wherein the deletion maintains protease activity of the mature form of SEQ ID NO: 2 with the carboxy terminal deletion.

75. The engineered protease polypeptide of claim 74, wherein the carboxy terminal deletion comprises deletion of the Big1 domain.

76. (canceled)

77. The engineered protease polypeptide of claim 75, further comprising 1, 2, 3, 4, 5, 6, 7, 8, 9, or up to 10 amino acid deletions of the carboxy terminus at amino acid residue 413 of SEQ ID NO: 2, wherein the further amino acid deletion(s) maintains proteolytic activity of the mature form of SEQ ID NO: 2 having the further amino acid deletions.

78. The engineered protease polypeptide of claim 74, wherein the mature form has an amino terminus at amino acid residue 128 or 135 of SEQ ID NO: 2.

79. (canceled)

80. A recombinant polynucleotide comprising a polynucleotide sequence encoding an engineered protease polypeptide of claim 1.

81-85. (canceled)

86. An expression vector comprising a recombinant polynucleotide of claim 80.

87. (canceled)

88. (canceled)

89. A host cell comprising an expression vector of claim 86.

90. (canceled)

91. A method of producing an engineered protease polypeptide, comprising culturing a host cell of claim 89 under suitable conditions such that the encoded engineered protease is expressed or produced.

92. (canceled)

93. (canceled)

94. A method of preparing a proteolytically active protease polypeptide comprising incubating an engineered protease polypeptide of claim 1 under suitable conditions such that the proteolytically active protease polypeptide or active protease is produced.

95-97. (canceled)

98. A pharmaceutical composition comprising an engineered protease polypeptide of claim 1.

99-104. (canceled)

105. A method of treating a disease or condition associated with a deficiency in pancreatic enzymes, the method comprising administering to a subject in need thereof an effective amount of an engineered protease polypeptide of claim 1.

106-111. (canceled)
